(19) United States
(12) Patent Application Publication
(10) Pub. No.: US 2010/0183633 A1
(43) Pub. Date: Jul. 22, 2010

(54) INTERLEUKIN 6 AND TUMOR NECROSIS FACTOR ALPHA AS BIOMARKERS OF JNK INHIBITION

(75) Inventors: Roger J. DAVIS, Princeton, MA (US); Guadalupe SABIO BUZO, Madrid (ES); Madhumita DAS, Shrewsbury, MA (US)

Correspondence Address: FISH & RICHARDSON PC, P.O. BOX 1022, MINNEAPOLIS, MN, 55440-1022, US

(73) Assignee: UNIVERSITY OF MASSACHUSETTS, Boston, MA (US)

(21) Appl. No.: 12/631,335

(22) Filed: Dec. 4, 2009

Related U.S. Application Data
(60) Provisional application No. 61/119,917, filed on Dec. 4, 2008.
(60) Provisional application No. 61/119,923, filed on Dec. 4, 2008.

(51) Int. Cl. A61K 39/395; C12Q 1/68; G01N 33/53; C07K 16/00

(52) U.S. Cl. 424/178.1; 435/6; 435/7.1; 530/391.7

(57) ABSTRACT

Biomarkers for JNK inhibition are described that can be used for monitoring effectiveness of JNK inhibitors and monitoring treatment with JNK inhibitors.

### CROSS-REFERENCE TO RELATED APPLICATIONS

This application claims priority to U.S. Application Ser. No. 61/119,917, filed Dec. 4, 2008, and U.S. Application Ser. No. 61/119,923, filed Dec. 4, 2008. The disclosures of the prior applications are incorporated by reference in their entirety.

### STATEMENT AS TO FEDERALLY SPONSORED RESEARCH

Funding for the work described herein was provided by the federal government (National Institutes of Health, Grant No. R01-CA065861), which has certain rights in the invention.

### TECHNICAL FIELD

The invention relates to biomarkers of JNK inhibition, and more particularly, to the use of interleukin 6 (IL6) and tumor necrosis factor alpha (TNFα) as biomarkers for JNK inhibition.

### BACKGROUND

The c-Jun NH2-terminal kinases (JNKs) phosphorylate and activate members of the activator protein-1 (AP-1) transcription factor family and other cellular factors implicated in regulating altered gene expression, cellular survival and proliferation in response to cytokines and growth factors, noxious stimuli, and oncogenic transformation. Because these events are commonly associated with the pathogenesis of a number of human diseases, JNK inhibitors have been developed to treat inflammatory, vascular, neurodegenerative, metabolic, and oncological diseases in humans. See, Manning and Davis, Nat. Rev. Drug Discov., 2, 554 (2003); and Borgoyevitch and Arthur, Biochim. Biophys. Acta, 1784, 76 (2008). As JNKs activate such a wide range of proteins, a need exists for biomarkers that can be used to monitor JNK inhibition.

### SUMMARY

The invention is based, in part, on the discovery that IL6 and TNFα can serve as biomarkers for JNK inhibition as levels of IL6 and TNFα can decrease upon treatment with a JNK inhibitor. In particular, as described herein, JNK1-deficient adipose tissue causes reduced diet-induced expression of IL6 and prevents diet-induced insulin resistance. In addition, JNK is essential for the development of hepatitis. Mice with JNK deficiency in hepatocytes are not protected against concavalin A- and lipopolysaccharide (LPS)-induced models of hepatitis, while mice with JNK deficiency in hematopoietic cells are protected in such models of hepatitis and exhibit decreased expression of TNFα. JNK inhibitors can be used for treating hepatitis and type 2 diabetes. IL6 and TNFα can be monitored to determine effectiveness of JNK inhibitors, monitor treatment with JNK inhibitors, as well as to identify JNK inhibitors in vitro. Levels of IL6 and TNFα are readily detectable and quantifiable in biological samples (e.g., serum samples).

In one aspect, this disclosure features a method of monitoring JNK inhibition in a subject being treated with a JNK inhibitor. The method includes obtaining a biological sample from a subject being treated with a JNK inhibitor; determining the level of IL6 or TNFα in the biological sample; and assessing a level of JNK inhibition based on the level of IL6 or TNFα in the biological sample. The JNK inhibitor can be an anthrapyrazolone compound, a peptide, an antisense oligonucleotide, or an siRNA. The method further can include comparing the level of IL6 or TNFα in the biological sample to a control level of IL6 or TNFα, wherein a decrease in the level of IL6 or TNFα in the subject relative to that of the control level is indicative of a positive response to the therapy in the subject. The control level can be the level of IL6 or TNFα in the subject before treatment with the JNK inhibitor or can be the level of IL6 or TNFα in a control population. The biological sample can be one or more of whole blood, plasma, serum, and adipose tissue. The level of IL6 or TNFα can be detected immunologically. For example, the level of IL6 or TNFα can be detected using a monoclonal antibody such as a monoclonal antibody attached to a solid substrate. The level of IL6 or TNFα can be determined using a capture antibody and a detection antibody, wherein the detection antibody includes a label (e.g., biotin, an enzyme, a radioisotope, or a fluorophore such as fluorescein, fluorescein isothiocyanate (FITC), phycoerythrin (PE), allophycocyanin (APC), or peridinin chlorophyll protein (PerCP)). The capture antibody can be attached to a solid substrate (e.g., a bead or a microtiter plate). The capture antibody can be a polyclonal antibody.

This disclosure also features a method of identifying a JNK inhibitor. The method includes contacting adipocytes in a culture medium with a test compound and monitoring expression of IL6 in the adipocytes. The test compound is identified as a JNK inhibitor if the expression of IL6 in the presence of the test compound is decreased relative to the expression of IL6 in the absence of the test compound. Expression of IL6 can be monitored by determining a level of IL6 protein in the culture medium, or determining a level of mRNA encoding IL6 in the adipocytes.

In another aspect, this disclosure features an article of manufacture for monitoring treatment with a JNK inhibitor. The article of manufacture can include reagents for measuring the level of IL6 in a biological sample from the patient, wherein the reagents are attached to a solid phase. The article of manufacture further can include a reagent for measuring the level of tumor necrosis factor alpha (TNFα), interferon gamma (IFNγ), or macrophage migration inhibiting factor-1 (MIF-1).

A composition also is featured that includes a JNK inhibitor linked to a monoclonal antibody having binding affinity for an epitope on an adipocyte. For example, the JNK inhibitor can be an anthrapyrazolone compound or a peptide. This disclosure also features methods of making targeted JNK inhibitors. Such methods can include linking a JNK inhibitor to a monoclonal antibody having binding affinity for an epitope on an adipocyte or linking a JNK inhibitor to an adipose homing protein.

In yet another aspect, this disclosure features a method for treating a patient having type 2 diabetes. The method includes administering to the patient an amount of a JNK inhibitor effective to increase insulin sensitivity in the patient; and monitoring IL6 levels in the serum of the patient to determine the efficacy of the treatment. The method further can include adjusting the amount of the JNK inhibitor administered to the patient based on the monitoring. The JNK inhibitor can be targeted to adipose tissue.

This disclosure also features an article of manufacture for monitoring treatment with a JNK inhibitor. The article of manufacture includes reagents for measuring the level of TNFα in a biological sample from the patient, wherein the reagents are attached to a solid phase. The article of manufacture further can include a reagent for determining the level of IFNγ, interleukin 2 (IL2), interleukin 4 (IL4), or IL6.

This disclosure also features a method for treating a patient having hepatitis. The method includes administering to the patient an amount of a JNK inhibitor effective to increase liver function in the patient, and monitoring TNFα levels in the serum of the patient to determine the efficacy of the treatment. The method can include adjusting the amount of the JNK inhibitor administered to the patient based on the monitoring.

This disclosure also features a method for treating a patient having type 2 diabetes that includes administering to the patient an amount of a JNK inhibitor effective to increase insulin sensitivity in the patient, wherein the JNK inhibitor is targeted to adipose tissue. The method further can include monitoring IL6 levels in the serum of the patient to determine the efficacy of the treatment. The JNK inhibitor can be conjugated to a monoclonal antibody having binding affinity for an epitope on an adipocyte.

In another aspect, this disclosure features a method for treating a patient having a disorder treatable with a JNK inhibitor. The method includes (a) administering to the patient an amount of the JNK inhibitor; (b) determining the level of JNK inhibition based on the level of IL6 or TNFα in a biological sample from the patient; and (c) administering an amount of JNK inhibitor different from (a) if the level of IL6 or TNFα determined in (b) indicates that more or less JNK inhibition in the patient is required. The disorder can be type 2 diabetes or hepatitis.

This disclosure also features compositions described herein for use as a medicament. For example, this disclosure features compositions for use in treating metabolic syndrome (e.g., type 2 diabetes) or inflammatory conditions (e.g., hepatitis). In another aspect, this disclosure features use of compositions described herein for the manufacture of a medicament for treatment of metabolic syndrome (e.g., type 2 diabetes) or inflammatory conditions (e.g., hepatitis).

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and from the claims.

### DESCRIPTION OF DRAWINGS

FIG. 1A is a schematic depicting the strategy for creating a conditional Jnk1 allele (Jnk1^{f}) using loxP sites that flank exon 7. PCR primers (5′-CCTCAGGAAGAAAGGGCTTATTTC-3′ and 5′-GAACCACTGTTCCAATTTCCATCC-3′, SEQ ID NO:1 and 2, respectively) that can be used to distinguish between the Jnk1⁺, Jnk1^{f}, and ΔJnk1 alleles are indicated.

FIG. 1B is a representation of an agarose gel separating PCR products that were produced using genomic DNA isolated from adipose tissue and amplimers that target introns 6 and 7. The analysis of adipose tissue from Fabp4-Cre⁺Jnk1^{+/-}(F^{WT}) and Fabp4-Cre⁺Jnk1^{f/-}(F^{KO}) mice is presented.

FIG. 1C is a representation of a gel in which macrophage genomic DNA isolated from F^{WT}, F^{KO}, Jnk1^{f/-}, Lyzs-Cre⁺Jnk1^{+/-}(M^{WT}), and Lyzs-Cre⁺Jnk1^{f/-}(M^{KO}) mice was analyzed for the indicated Jnk1 alleles.

FIGS. 2A-1 to 2A-6 are bar graphs depicting mass (lean, fat, and total, mean±SD; n=8); blood glucose concentration (mice fed ad libitum or fasted overnight, mean±SD; n=14); and IL6 concentration (mice fasted overnight, mean±SD, n=14) in Lyzs-Cre⁺Jnk1^{+/+}(M^{WT}) and Lyzs-Cre⁺Jnk1^{f/f}(M^{KO}) mice fed a normal chow diet (ND) or a high fat diet (HF). No statistically significant differences between M^{KO} mice and M^{WT} mice were detected (P>0.05).

FIGS. 2B-1 and 2B-2 are graphs depicting blood glucose concentration at the indicated times (mean±SD; n=14) during a glucose tolerance test (GTT) (B-1) and an insulin tolerance test (ITT) (B-2). No statistically significant differences between M^{KO} mice and M^{WT} mice were detected (P>0.05).

FIG. 3A is a bar graph of the expression of Jnk1 mRNA in blood as examined by quantitative RT-PCR analysis (Taqman®) in bone marrow chimeric mice. Results are presented as relative mRNA expression (mean±SD; n=6). Control studies were performed to detect Gapdh mRNA. The amount of Jnk1 mRNA was significantly reduced in the blood of mice transplanted with Jnk1^{-/-} bone marrow compared with Jnk1^{+/+} bone marrow (asterisk, P<0.01).

FIGS. 3B-1 and 3B-2 are bar graphs of blood glucose (mg/dL) in bone marrow chimeric mice fasted overnight or fed (mean±SD; n=10). No statistically significant differences between mice transplanted with Jnk1^{-/-} or Jnk1^{+/+} bone marrow were detected (P>0.05).

FIG. 3C is a graph of the body mass of bone marrow chimeric mice during the 16 wk diet using a standard chow (ND) or a high fat (HF) diet (mean±SD; n=10). No statistically significant differences were detected (P>0.05) between mice transplanted with Jnk1^{-/-} or Jnk1^{+/+} bone marrow.

FIGS. 3D-3E are graphs of blood glucose concentration during a GTT and pyruvate challenge, respectively, using bone marrow chimeric mice (mean±SD; n=10). FIG. 3F is a bar graph of the AUC for blood glucose concentration during an ITT using bone marrow chimeric mice (mean±SD; n=10). No statistically significant differences were detected (P>0.05) between mice transplanted with Jnk1^{-/-} or Jnk1^{+/+} bone marrow during the GTT, pyruvate challenge, or ITT.

FIGS. 4A-1 and 4A-2 are bar graphs depicting the expression of Jnk1 mRNA in adipose tissue (A-1, epididymal fat; A-2, brown fat) as examined by quantitative RT-PCR analysis (Taqman®). Expression is presented as relative mRNA expression (mean±SD; n=5). The data are normalized for the amount of Gapdh mRNA in each sample. The amount of Jnk1 mRNA was significantly reduced in the adipose tissue of F^{KO} mice compared to F^{WT} mice (*, P<0.01).

FIG. 4B is a series of representations of immunoblots to detect JNK1 expression in adipose tissue, liver, muscle (quadriceps), and macrophages isolated from F^{WT}, F^{KO}, and Jnk1^{-/-} mice. Control immunoblots were performed using antibodies to Actin and Tubulin.

FIG. 4C is a representation depicting JNK activity as measured by a kinase activity (KA) assay using ATP[γ-³²P] and cJun as substrates. F^{KO} and F^{WT} mice were maintained on a standard chow diet (ND) or on a high fat diet (HF) for 16 wk. Protein extracts were prepared from epididymal fat, muscle (quadriceps), and liver. Equal amounts of cell extract prepared from F^{WT} and F^{KO} mice, confirmed by immunoblot analysis using an antibody to Tubulin, were used to measure JNK activity in the KA.

FIG. 5A is a graph of the body mass of Fabp4-Cre⁺Jnk1^{+/-}(F^{WT}) and Fabp4-Cre⁺Jnk1^{f/-}(F^{KO}) mice fed a normal chow diet (ND) or a high fat diet (HF) (mean±SD; n=14). No statistically significant differences between F^{KO} mice and F^{WT} mice were detected (P>0.05).

FIG. 5B-1 is a series of representations of sections of epididymal fat pads stained with H&E. FIG. 5B-2 is a bar graph of the mass of epididymal fat pads (right panel) of mice fed a standard chow or a high fat diet for 16 wk (mean±SD; n=14). No statistically significant differences were detected (P>0.05) between F^{KO} mice and F^{WT} mice.

FIGS. 5C-1 to 5C-4 are a series of bar graphs of the mass of brown fat, heart, liver, and muscle (quadriceps) of mice fed a chow or a high fat diet for 16 wk (mean±SD; n=14). No statistically significant differences were detected (P>0.05) between F^{KO} mice and F^{WT} mice.

FIGS. 5D-1 and 5D-2 are bar graphs of the lean mass and whole body fat mass of mice fed a chow or high fat diet (3 wks) as non-invasively measured by ¹H-MRS (mean±SD; n=8). No statistically significant differences between F^{KO} mice and F^{WT} mice were detected (P>0.05).

FIGS. 6A-6F are bar graphs of cholesterol, triglycerides, HDL, LDL, glycerol, and free fatty acids (FFA), respectively, in F^{WT} and F^{KO} mice fed a normal chow diet (ND) or a high fat diet (16 wk). The mice were fasted overnight and blood was taken for the measurement of the recited components. No statistically significant differences between F^{KO} mice and F^{WT} mice were detected (P>0.05) (mean±SD; n=10).

FIGS. 7A-7B are graphs depicting blood glucose concentration (mean±SD; n=14) during a GTT and ITT, respectively, in F^{KO} and F^{WT} maintained on a standard chow diet (ND) or on a high fat diet (HF) for 16 wk.

FIG. 7C is a graph depicting glucose-induced insulin release. Mice fasted overnight were injected intraperitoneally with glucose (2 mg/g). Blood insulin concentration was measured at the indicated times (mean±SD; n=14). No statistically significant differences between F^{KO} and F^{WT} mice were detected (P>0.05).

FIGS. 7D-7L are bar graphs depicting steady-state glucose infusion rates to maintain euglycemia during the hyperinsulinemic-euglycemic clamps (D), insulin-stimulated whole body glucose turnover (E), whole body glycolysis (F), basal hepatic glucose production (HGP) (G), insulin-stimulated rates of HGP during clamps (H), hepatic insulin action (I), glycogen synthesis (J), insulin levels (K), and glucose levels (L) in F^{KO} and F^{WT} mice maintained on a standard chow diet or on a high fat (HF) diet for 3 wk. In FIG. 7I, hepatic insulin action is expressed as insulin-mediated percent suppression of basal HGP. For FIG. 7D-7J, the data presented are the mean±SE for 6 to 8 experiments. Statistically significant differences are indicated (*, P<0.05; **, P<0.01; ***, P<0.001). For FIG. 7K-7L, resting blood insulin and glucose were examined in mice that were fasted overnight (mean±SD, n=10). Statistically significant differences between F^{KO} mice and F^{WT} mice are indicated (*, P<0.01).

FIG. 8A is a graph of blood glucose concentration during a pyruvate challenge test in F^{WT} and F^{KO}. Statistically significant differences between F^{KO} mice and F^{WT} mice are indicated (*, P<0.01). The data demonstrate that feeding a HF diet suppresses pyruvate induced hepatic gluconeogenesis in F^{WT} mice, but not in F^{KO} mice.

FIGS. 8B-8E are bar graphs of the relative mRNA of the indicated gene as assessed by quantitative RT-PCR (mean±SD; n=8) using total RNA isolated from the liver. The data are normalized for the amount of actin mRNA in each sample. Significant differences in the expression of Pepck, glucose 6 phosphatase, and glucose 6 kinase mRNA detected between F^{WT} and F^{KO} mice are indicated (*, P<0.05). The data demonstrate that altered expression of Pepck, glucose 6 phosphatase, or glucose 6 kinase does not account for the failure of a HF diet to suppress hepatic gluconeogenesis in F^{KO} mice. It is possible that the reduced steatosis observed in HF diet-fed in F^{KO} mice, compared with F^{WT} mice, contributes to the effects on adipose tissue specific JNK1-deficiency on pyruvate-induced gluconeogenesis (FIG. 10A). No significant differences (P<0.05) in the expression of myeloid-specific Cd68 mRNA were detected between livers isolated from F^{WT} and F^{KO} mice (FIG. 8E). These data indicate that adipose tissue-specific JNK1-deficiency does not affect myeloid cell infiltration of the liver.

FIGS. 9A-1 to 9A-2 are bar graphs of the relative mRNA expression of IL6 (A-1) and TNFα (A-2) in adipose tissue as examined by quantitative RT-PCR analysis (Taqman®) (mean±SD; n=6). Statistically significant differences between F^{KO} mice and F^{WT} mice are indicated (*, P<0.01).

FIG. 9B is a representation of an immunoblot to detect AKT and phospho-AKT. F^{WT} and F^{KO} mice were fasted overnight and treated with insulin (1.5 U/kg body mass) by intraperitoneal injection. The epididymal fat pads were isolated after 30 min and examined by immunoblot analysis as set forth in Example 1.

FIGS. 9C-1 to 9C-4 are bar graphs of the amount of IL6, TNFα, leptin and resistin in plasma from mice fasted overnight (mean±SD; n=10). Statistically significant differences between F^{KO} mice and F^{WT} mice are indicated (*, P<0.05).

FIG. 10A is a representation of histological sections of liver stained with H&E or Oil Red-0 from F^{WT} and F^{KO} mice fed a standard chow diet or a high fat diet for 16 wk.

FIG. 10B is a series of representations of immunoblots to detect AKT, phospho-AKT, JNK1/2, and tubulin. Mice were fasted overnight and treated with insulin (1.5 U/kg bodymass) by intraperitoneal injection. Livers were isolated after 30 min and examined by immunoblot analysis as set forth in Example 1.

FIG. 10C is a representation of an immunoblot to detect expression of SOCS3 in the liver. Control immunoblots were performed using an antibody to Tubulin.

FIG. 10D is a representation of an immunoblot to detect tyrosine phosphorylation and expression of the insulin receptor (IR) and IRS1 in the liver.

FIG. 10E is a representation of an immunoblot to detect expression of SOCS3 and tubulin (control). F^{WT} and F^{KO} mice were fed a HFD (16 wks). F^{KO} mice were treated with IL6 (1 μg/kg) by subcutaneous injection. At 90 mins post-injection, the blood IL6 concentration was 44±9.1 pg/ml (mean±SD; n=8), the liver was isolated, and the expression of SOCS3 and Tubulin was examined by immunoblot analysis as set forth in Example 1.

FIG. 10F is a graph of the glucose concentration during an ITT in which mice were treated by subcutaneous injection with IL6 (1 μg/kg) and then treated (after 90 mins) with insulin (0.75 mU/g) by intraperitoneal injection. Statistically significant differences between F^{KO} and F^{WT} mice are indicated (*, P<0.05; **, P<0.01).

FIGS. 10G and 10H are representations of immunoblots to detect AKT, phospho-AKT, and tubulin in liver (G) and epididymal fat pads (H). HFD-fed F^{KO} mice were subcutaneously injected with IL6 (1 μg/kg) and after 90 mins, intravenously injected with insulin (0.3 mU/g). The liver (G) and epididymal fat pads (H) were isolated after 5 mins and examined by immunoblot analysis as set forth in Example 1.

FIG. 11 is a representation of an immunoblot to detect phospho-AKT, AKT, and GAPDH. F^{WT} and F^{KO} mice were fasted overnight and then intraperitoneally injected with insulin (1.5 U/kg body mass). Muscle (quadriceps) was isolated 30 min post-treatment with insulin and examined by immunoblot analysis as set forth in Example 1.

FIGS. 12A-12F are bar graphs of the plasma levels of different cytokines in F^{WT} and F^{KO}. Mice fed a chow diet or a high fat diet for 16 wk and then fasted overnight before measurement of the cytokines. No statistically significant difference in the serum concentration of ILL IL2, IL4, IL5, IL10, and IL12 was detected between F^{WT} and F^{KO} mice (P>0.05).

FIG. 13A is a representation of an immunoblot to detect adiponectin in F^{WT} and F^{KO} mice. High molecular weight adiponectin in the blood was examined by immunoblot analysis of plasma examined by native gel electrophoresis. Each lane represents the analysis of serum from one mouse. Feeding a HFD caused a small decrease in serum adiponectin in F^{WT} mice, but not in F^{KO} mice.

FIGS. 13B-13I are bar graphs of the relative expression of mRNA from adiponectin, Pparγ, lysozyme, Rbp4, Mcp-1, Mif, Steap4, and Pbef1 genes, respectively, as examined by quantitative RT-PCR (mean±SD; n=6). No significant differences (P>0.05) in the expression of Rbp4, Pbef1, or Mcp-1 mRNA were detected between F^{WT} and F^{KO} mice. In contrast, the HFD-induced increase in Mif and Steap4 mRNA was reduced in F^{KO} compared with F^{WT} mice (*, P<0.01). This reduction in Mif and Steap4 mRNA expression is consistent with previous reports indicating that Mif expression is JNK1-dependent (Tuncman et al., Proc Natl Acad Sci USA 103, 10741 (2006)) and that Steap4 expression is IL6-dependent (Fasshauer et al., FEBS Lett 560, 153 (2004)). Marker gene expression analysis did not indicate differences in adipose tissue differentation (Pparγ) or macrophage infiltration (Lysozyme) between HFD-fed F^{WT} and F^{KO} mice.

FIG. 14A is a bar graph of the relative level of IL13 mRNA in epididymal fat pads from F^{WT} and F^{KO} mice as assessed by quantitative Taqman® RT-PCR analysis (mean±SD; n=6). The data are normalized for the amount of Gapdh mRNA in each sample. FIG. 14B is a bar graph of the concentration of IL13 in the blood (mean±SD; n=10) in F^{WT} and F^{KO} mice. No significant differences between F^{WT} and F^{KO} mice were detected (P>0.05) in the expression of the IL13 mRNA in adipose tissue or IL13 protein in blood.

FIG. 15 is a bar graph of the relative expression of Cd68 mRNA in epididymal fat pads as assessed by quantitative Taqman® RT-PCR analysis (mean±SD; n=6). The data are normalized for the amount of Gapdh mRNA in each sample. No significant differences in the expression of Cd68 gene between F^{WT} and F^{KO} mice were detected (P>0.05). CD68 is expressed selectively in myeloid cells. These data indicate that there is no difference in the macrophage infiltration of adipose tissue between F^{WT} and F^{KO} mice.

FIGS. 16A-1 and A-2 and 16B-1 and B-2 are graphs of the amount of TNFα or IL6 in culture medium at the indicated time points (mean±SD; n=5) from bone marrow-derived macrophages from (A) WT and Jnk1^{-/-} mice or (B) F^{WT} and F^{KO} mice stimulated with 0.5 mM palmitate. Statistically significant differences between F^{WT} and F^{KO} mice are indicated (*, P<0.05; **, P<0.01).

FIG. 17A is a series of representations of H&E-stained liver sections from wild-type, Jnk1^{-/-}, and Jnk2^{-/-} mice treated intravenously (8 hrs) with ConA or solvent (saline). The amount of liver damage was quantitated (see FIG. 2A).

FIGS. 17B-1 and 17B-2 are two bar graphs of serum transaminase activity (ALT and AST) in control and JNK-deficient mice after treatment (8 hrs) with ConA or solvent (saline) (mean±SD; n=6). No statistically significant differences between wild-type and JNK-deficient mice were detected.

FIGS. 17C-1 to 17C-8 are bar graphs of the amount of serum cytokines (ILL IL2, IL4, IL6, IL10, IL12, TNFα, and IFNγ) at 8 hrs. post-treatment with ConA as measured by ELISA (mean±SD; n=4). Statistically significant differences between wild-type and JNK-deficient mice are indicated (*, P<0.05).

FIGS. 18A-18D are bar graphs quantitating hepatic damage caused by ConA and LPS (mean % area ±SD; n=10). Statistically significant differences between wild-type and JNK-deficient mice are indicated (*, P<0.01).

FIG. 19A is a series of representations of H&E-stained liver sections from wild-type, Jnk1^{-/-}, and Jnk2^{-/-} mice treated intravenously (8 hrs) with LPS plus GalN or solvent (saline). The amount of liver damage was quantitated (see FIG. 2B).

FIG. 19B is a bar graph of serum transaminase activity (ALT and AST) in control and JNK-deficient mice after treatment (8 hrs) with LPS plus GalN or solvent (saline) (mean±SD; n=6). Statistically significant differences between wild-type and JNK-deficient mice are indicated (*, P<0.05).

FIGS. 19C-1 to 19C-5 are graphs of the amount of serum cytokines (IFNγ, TNFα, GM-CSF, IL6, IL10) post-treatment with LPS plus GalN as measured by ELISA (mean±SD; n=6).

FIGS. 20A and 20B are bar graphs of the concentration of TNFα in the culture medium at 4 hr (ConA) or 8 hr (LPS) post-treatment, respectively, of bone marrow-derived macrophages isolated from wild-type, Jnk1^{-/-}, and Jnk2^{-/-} mice. The cells (1×10⁶) were cultured in 1.0 ml medium and treated without and with 2.5 μg of ConA or 1.0 μg LPS.

FIGS. 20C and 20D are bar graphs of the concentration of TNFα in the culture medium at 24 hr (ConA) or 8 hr (LPS) post-treatment, respectively, of bone marrow-derived macrophages isolated from polyIC-treated control mice (Mx1-Cre⁺) and JNK-deficient (Jnk1^{f/f}Jnk2^{-/-}Mx1-Cre⁺) mice.

FIG. 20E is a bar graph of the concentration of TNFα in the culture medium from CD4 T cells (5×10⁵) isolated from wild-type, Jnk1^{-/-}, and Jnk2^{-/-} mice, cultured in 0.5 ml medium, and treated without and with 1.25 μg of ConA plus 0.5 μg anti-CD28 (BD-Pharmingen). The concentration of TNFα in the culture medium was measured by ELISA at 48 hr post-treatment.

FIG. 20F is a bar graph of the concentration of TNFα in the culture medium from CD4 T cells isolated from polyIC-treated control mice (Mx1-Cre⁺) and JNK-deficient (Jnk1^{f/f}Jnk2^{-/-}Mx1-Cre⁺) mice. The concentration of TNFα in the culture medium was measured by ELISA at 48 hr post-treatment with 1.25 μg of ConA plus 0.5 μg anti-CD28. The data presented represent the mean±SD (n=3). Statistically significant differences between control and JNK-deficient cells are indicated (*, P<0.05).

FIG. 21A is a representation of an immunoblot to detect JNK1/2, cFLIP, and α-Tubulin in liver extracts from polyIC-treated control mice (Mx1-Cre⁺) and JNK-deficient mice (Jnk1^{f/f}Jnk2^{-/-}Mx1-Cre⁺) aged 4 weeks and then treated intravenously (8 hrs) with ConA or solvent (saline).

FIG. 21B is a representation of H&E-stained liver sections prepared from control and JNK1/2-deficient mice treated (8 hrs) with ConA or solvent (saline) are presented. The amount of liver damage was quantitated (see FIG. 18C).

FIG. 21C is a bar graph of serum transaminase activity in control and JNK1/2-deficient mice treated (8 hrs) with ConA or solvent (saline) (mean±SD; n=6). Statistically significant differences between wild-type and JNK1/2-deficient mice are indicated (*, P<0.01).

FIGS. 21D-1 and 21D-2 are bar graphs depicting the relative mRNA expression of cJun, JunB, JunD, cFos (left panel), or Tnfα (right panel) in the liver of control and JNK1/2-deficient mice treated (8 hrs) with ConA or solvent (saline). The mRNA expression was normalized to the amount of Gapdh mRNA and presented as the mean±SD (n=6). Statistically significant differences between wild-type and JNK1/2-deficient mice are indicated (*, P<0.01).

FIGS. 22A and 22B are Kaplan-Meier survival curves of the indicated mice. In FIG. 22A, polyIC-treated control mice (Mx1-Cre⁺) and JNK-deficient (Jnk1^{f/f}Jnk2^{-/-}Mx1-Cre⁺) mice were treated with ConA. Kaplan-Meier analysis of the survival of groups of 12 mice per genotype demonstrated that the JNK-deficient mice exhibited reduced mortality compared with control mice (log-rank test; P<0.005). In FIG. 22B, control mice (Alb-Cre⁺) and JNK-deficient (Jnk1^{f/f}Jnk2^{-/-}Alb-Cre⁺) mice were treated with ConA. Kaplan-Meier analysis of the survival of groups of 12 mice per genotype demonstrated that there was no statistically significant difference between the mortality of the control and JNK-deficient mice (log-rank test; P>0.05).

FIGS. 23-1 to 23-10 are a series of graphs of the amount of the indicated serum cytokine from polyIC-treated control mice (Mx1-Cre⁺) and JNK-deficient mice (Jnk1^{f/f}Jnk2^{-/-}Mx1-Cre⁺) post-treatment with ConA (mean±SD; n=7). Statistically significant differences between wild-type and JNK1/2-deficient mice are indicated (*, P<0.05; **, P<0.01).

FIG. 24A is a series of representations of H&E-stained liver sections from polyIC treated control mice (Mx1-Cre⁺) and JNK-deficient mice (Jnk1^{f/f}Jnk2^{-/-}Mx1-Cre⁺) treated (8 hrs) with LPS plus GalN or solvent (saline). The amount of liver damage was quantitated (see FIG. 2D).

FIG. 24B is a bar graph of the serum transaminase activity (ALT and AST) in control and JNK-deficient mice after treatment (8 hrs) with LPS plus GalN or solvent (saline) (mean±SD; n=6). Statistically significant differences between wild-type and JNK1/2-deficient mice are indicated (*, P<0.01).

FIG. 24C is a representation of an immunoblot to detect cFLIP and α-Tubulin in liver extracts from control and JNK-deficient mice at 8 hrs post-treatment with LPS plus GalN or solvent (saline). The numbers on the left indicate the electrophoretic mobility of protein standards (kDa).

FIG. 24D is a bar graph of the relative mRNA expression of Gapdh, cJun, JunB, JunD, cFos, and Tnfα in the liver of control and JNK-deficient mice after treatment (8 hrs) with LPS plus GalN or solvent (saline). The mRNA expression in each sample was normalized to the amount of Gapdh mRNA and presented as the mean±SD (n=6). Statistically significant differences between wild-type and JNK1/2-deficient mice are indicated (*, P<0.01).

FIG. 24E is a bar graph of the serum TNFα concentration after treatment (1 hr) with LPS plus GalN or saline (mean±SD; n=6). Statistically significant differences between mice transplanted with control and JNK1/2-deficient bone marrow are indicated (*, P<0.01).

FIG. 25A and FIG. 25B are Kaplan-Meier survival curves of the indicated mice. In FIG. 25A, polyIC-treated control mice (Mx1-Cre⁺) and JNK-deficient (Jnk1^{f/f}Jnk2^{-/-}Mx1-Cre⁺) mice were treated with LPS plus GalN. Kaplan-Meier analysis of the survival of groups of 15 mice per genotype demonstrated that the JNK-deficient mice exhibited reduced mortality compared with control mice (log-rank test; P<0.01). In FIG. 25B, control mice (Alb-Cre⁺) and JNK-deficient (Jnk1^{f/f}Jnk2^{-/-}Alb-Cre⁺) mice were treated with TNFα plus GalN. Kaplan-Meier analysis of the survival of groups of 12 mice per genotype demonstrated that there was no statistically significant difference between the mortality of the control and JNK-deficient mice (log-rank test; P>0.05).

FIG. 26A is a representation of an immunoblot to detect JNK1/2 and α-Tubulin in liver extracts prepared from control mice (Alb-Cre⁺) and mice with hepatocyte-specific JNK-deficiency (Jnk1^{f/f}Jnk2^{-/-}Alb-Cre⁺).

FIGS. 26B and 26C are representations of H&E-stained (B) and TUNEL-stained (C) liver sections prepared from control and JNK1/2-deficient mice treated intravenously (8 hrs) with ConA or solvent (saline).

FIG. 26D is a bar graph of the serum transaminase activity in control and JNK-deficient mice after treatment (8 hrs) with ConA or solvent (saline) (mean±SD; n=6). No statistically significant differences between wild-type and JNK1/2-deficient mice were detected.

FIGS. 26E-1 to 26E-3 are bar graphs of the relative mRNA expression of cJun, JunB, JunD, cFos, p53, p21, Mdm2, Bax, Puma, and Tnfα the liver of control and JNK-deficient mice after treatment (8 hrs) with ConA or solvent (saline). The mRNA expression was normalized to the amount of Gapdh mRNA and presented as the mean±SD (n=6). No statistically significant differences between wild-type and JNK1/2-deficient mice were detected.

FIGS. 27A-1 to 27A-10 are graphs of the amount of serum cytokines (IL1, IL2, IL4, IL5, IL6, IL10, IL12, GM-CSF, TNFα, and IFNγ) in control mice (Alb-Cre⁺) and mice with hepatocyte-specific JNK-deficiency (Jnk1^{f/f}Jnk2^{-/-}Alb-Cre⁺) post-treatment with ConA (mean±SD; n=8). Statistically significant differences between wild-type and JNK1/2-deficient mice are indicated (*, P<0.05).

FIGS. 27B-1 to 27B-4 are bar graphs of the phosphorylated/non-phosphorylated ratio of the indicated proteins in liver extracts at 8 hrs post-treatment of mice with ConA or saline (mean±SD; n=5). Statistically significant differences between wild-type and JNK1/2-deficient mice are indicated (*, P<0.01).

FIGS. 28A-1 to 28A-3 are graphs of the amount of serum cytokines (TNFα, IL6, and IL10, respectively) in control mice (Alb-Cre⁺) and mice with hepatocyte-specific JNK-deficiency (Jnk1^{f/f}Jnk2^{-/-}Alb-Cre⁺) post-treatment with LPS plus GalN (mean±SD; n=7).

FIGS. 28B-1 to 28B-4 are bar graphs of the amount of total and phospho-JNK1/2, cJun, ERK1/2, p38 MAPK, and AKT in liver extracts at 8 hrs post-treatment of mice with LPS plus GalN or saline (mean±SD; n=5). Statistically significant differences between wild-type and JNK1/2-deficient mice are indicated (*, P<0.05).

FIG. 29A is a series of representations of H&E- and TUNEL-stained liver sections prepared from control (Alb-Cre⁺) and hepatocyte-specific JNK-deficiency (Jnk1^{f/f}Jnk2^{-/-}Alb-Cre⁺) treated intravenously (8 hrs) with LPS plus GalN or solvent (saline).

FIG. 29B is a bar graph of the serum transaminase activity in control and JNK1/2-deficient mice after treatment (8 hrs) with LPS/GalN or solvent (saline) (mean±SD; n=6). No statistically significant differences between wild-type and JNK1/2-deficient mice were detected.

FIG. 29C is a bar graph of the concentration of TNFα in the serum of control mice (Alb-Cre⁺) and hepatocyte-specific JNK-deficiency mice (Jnk1^{f/f}Jnk2^{-/-}Alb-Cre⁺) after treatment (1 hr) with LPS/GalN or solvent (saline) (mean±SD; n=6). No statistically significant differences between wild-type and JNK1/2-deficient mice were detected.

FIG. 29D is a representation of an immunoblot to detect cFLIP and α-Tubulin in liver extracts prepared from control mice (Alb-Cre⁺) and JNK1/2-deficient mice (Jnk1^{f/f}Jnk2^{-/-}Alb-Cre⁺).

FIGS. 29E-1 to 29E-3 are bar graphs depicting the relative mRNA expression of cJun, JunB, JunD, cFos, p53, p21, Mdm2, Bax, Puma, and Tnfα in the liver of control and JNK-deficient mice after treatment (8 hrs) with LPS/GalN or solvent (saline). The mRNA expression was normalized to the amount of Gapdh mRNA and presented as the mean±SD (n=6). No statistically significant differences between wild-type and JNK1/2-deficient mice were detected.

FIG. 30A is a representation of an immunoblot to detect JNK1/2 and α-Tubulin in bone marrow-derived macrophages prepared from polyIC-treated control mice (Mx1-Cre⁺) and JNK-deficient mice (Jnk1^{f/f}Jnk2^{-/-}Mx1-Cre⁺) then treated with ConA. FIGS. 30B-1 to 30B-3 are graphs of the amount of the indicated cytokines in the culture medium from the treated macrophages (mean±SD; n=7). Statistically significant differences between wild-type and JNK1/2-deficient mice are indicated (*, P<0.01).

FIGS. 30C and 30D are representations of immunoblots to detect capsase-cleaved PARP, cleaved caspase-3, and α-Tubulin in liver extracts from polyIC-treated control mice (Mx1-Cre⁺) and JNK-deficient mice (Jnk1^{f/f}Jnk2^{-/-}Mx1-Cre⁺) (C) or (Alb-Cre⁺) and mice with hepatocyte-specific JNK-deficiency (Jnk1^{f/f}Jnk2^{-/-}Alb-Cre⁺) (D) treated intravenously (8 hrs) with TNFα plus GalN or solvent (saline).

FIG. 30E is a bar graph of the serum transaminase activity in control and JNK1/2-deficient mice after treatment (8 hrs) with LPS/GalN or solvent (saline) (mean±SD; n=6). No statistically significant differences between wild-type and JNK1/2-deficient mice were detected.

FIG. 31 is a bar graph of the relative expression of IL22 mRNA in the liver of control mice (Mx1-Cre⁺) and JNK-deficient (Jnk1^{f/f}Jnk2^{-/-}Mx1-Cre⁺) treated without and with ConA. IL22 mRNA expression was examined 1 hr post-injection. The mRNA expression in each sample was normalized to the amount of Gapdh mRNA and presented as the mean±SD (n=6). No statistically significant differences between wild-type and JNK-deficient mice were detected (P>0.05).

FIG. 32A is a bar graph of the percent (%) of CD45.2 leukocytes at 6 months post-transplantation (mean±SD; n=3) in lethally-irradiated wild-type B6.SJL mice transplanted with an equal number of bone marrow cells isolated from polyIC-treated (4 weeks) control B6.SJL (CD45.1) mice plus Jnk1^{f/f}Jnk2^{-/-}Mx1-Cre⁺ or wildtype Mx1-Cre⁺ C57BL/6J (CD45.2) mice. Peripheral blood leukocytes were stained with antibodies to CD45.1 and CD45.2.

FIG. 32B is a bar graph of the relative Jnk1 mRNA expression in peripheral blood leukocytes of lethally-irradiated wild-type mice transplanted with bone marrow from polyIC-treated control mice (Mx1-Cre⁺) or JNK1/2-deficient mice (Jnk1^{f/f}Jnk2^{-/-}Mx1-Cre⁺) mice. FIG. 32C is a representation of a gel in which genomic DNA was genotyped by PCR analysis using amplimers to detect the Jnk1⁺, Jnk1^{f}, and Jnk1^{Δ} alleles.

FIG. 32D is a representation of an immunoblot to detect JNK1/2 and ERK1/2 in splenocytes from polyIC-treated control mice and JNK1/2-deficient mice.

FIG. 32E is a bar graph of the CD4, CD8, and B220 sub-populations of splenocytes as measured by flow cytometry.

FIG. 32F is a bar graph of serum transaminase activity (ALT and AST) (mean±SD; n=6) in mice at 6 months post-transplantation treated intravenously (8 hrs) with ConA or solvent (saline). Statistically significant differences between mice transplanted with control and JNK1/2-deficient bone marrow are indicated (*, P<0.01).

FIG. 32G is a bar graph of the relative expression of Tnfα in the liver of mice at 6 months post-transplantation that were treated intravenously with ConA or solvent (saline). Tnfα mRNA was measured at 8 hr. post-injection. The mRNA expression in each sample was normalized to the amount of Gapdh mRNA and presented as the mean±SD (n=6) (left panel). Statistically significant differences between mice transplanted with control and JNK1/2-deficient bone marrow are indicated (*, P<0.01).

FIG. 33A is a representation of an immunoblot to detect AKT and phosphoAKT in liver extracts from chow-fed L^{WT} and L^{KO} mice that were fasted overnight and administered glucose (2 g/kg) by intraperitoneal injection.

FIG. 33B is a graph of the blood glucose concentration of chow-fed L^{WT} and L^{KO} mice during glucose tolerance tests. The data presented represent the mean±SD (n=10˜15). Statistically significant differences are indicated (*, P<0.05; **, P<0.01).

FIG. 33C is a bar graph of the blood glucose concentration (mean±SD; n=10˜15) in chow-fed L^{WT} and L^{KO} mice after fasting overnight (left panel) or fed ad libitum (right panel). Statistically significant differences are indicated (***, P<0.001).

FIG. 34A is a bar graph of the blood insulin concentration (mean±SD; n=13˜15) in L^{KO} and L^{WT} mice. No statistically significant difference between L^{KO} and L^{WT} was detected.

FIG. 34B is a graph of the change in concentration of insulin in the blood (mean±SD; n=10-15) after administration of glucose (2 g/kg body mass). Statistically significant differences between L^{KO} and L^{WT} mice are indicated (*, P<0.01).

FIG. 34C is a graph of the C-peptide concentration (mean±SD; n=15) in the blood. No statistically significant difference between L^{KO} and L^{WT} was detected. Statistically significant differences between L^{KO} and L^{WT} are indicated (****, P<0.0001).

FIG. 34D is a graph of the change in C-peptide concentration in the blood (mean±SD; n=15) after chow-fed L^{KO} and L^{WT} mice were injected with human glucose (2 g/kg body mass). No statistically significant difference between L^{KO} and L^{WT} was detected

FIG. 34E is a graph of the concentration of human insulin in the blood (mean±SD; n=15) after chow-fed L^{KO} and L^{WT} mice were injected with human insulin (1.5 U/kg body mass).Statistically significant differences between L^{KO} and L^{WT} are indicated (*, P<0.05).

FIG. 34F is a bar graph of the expression of the insulin receptor and Ceacam-1 mRNA in the liver of mice fasted overnight. The expression of insulin receptor and Caecam-1 mRNA was normalized to the amount of 18S RNA in each sample (mean±SD; n=7). Statistically significant differences between L^{KO} and L^{WT} are indicated (*, P<0.05; ***, P<0.001).

FIGS. 35A to 35F are bar graphs of basal hepatic glucose production (HGP) (A); insulin-stimulated rate of HGP (B); hepatic insulin action, expressed as insulin-mediated percent suppression of basal HGP (C); insulin-stimulated whole body glucose turnover (D); whole body fat mass (measured using ¹H-MRS) (E); and whole body lean mass (F). The data presented are the mean±SE for 6˜8 experiments. Statistically significant differences between L^{KO} and L^{WT} mice are indicated (*, P<0.05).

FIG. 35G is a representation of immunoblots to detect IRS1 and phosphoSer-307 IRS1 and FIG. 35H is a representation of immunoblots to detect AKT and phosphoAKT after chow-fed L^{KO} and L^{WT} mice were administered insulin (0.3 U/kg body mass) by intravenous injection.

FIG. 36A is a representation of histological sections of the liver stained with Oil Red-O from chow-fed L^{WT} (left panel) and L^{KO} (right panel) mice that were fasted overnight.

FIG. 36B is a bar graph of the amount of hepatic triglyceride in mice fasted over night (mean±SD; n=10). Statistically significant differences between L^{KO} and L^{WT} are indicated (*, P<0.05).

FIG. 36C is a bar graph of the amount of hepatic lipogenesis in mice fasted 6 hours (mean±SD; n=8˜9). Statistically significant differences between L^{KO} and L^{WT} are indicated (*, P<0.05).

FIG. 36D is a bar graph of the expression of genes that encode lipogenic transcription factors and co-activators in the liver of chow-fed L^{KO} and L^{WT} mice that were fasted overnight. C/ebpα, C/ebpβ, Pgc1β, Ppary, and Srebp1 mRNAs were measured by quantitative RT-PCR assays and normalized to the amount of 18S RNA in each sample (mean±SD; n=7). Statistically significant differences between L^{KO} and L^{WT} are indicated (*, P<0.05; **, P<0.01).

FIG. 36E is a bar graph of the relative mRNA expression of genes encoding enzymes that promote lipogenesis in the liver of chowfed L^{KO} and L^{WT} mice that were fasted overnight. Fas, fatty acid synthase; Acsl1/4, acetyl-CoA synthetase long chain family member 1/4; Acacα/β, acetyl-CoA carboxylase α/β; Acot3, Acetyl-CoA thioesterase; Dgat1, Diacylglycerol O-acyltransferase homolog 1; Glyk, Glycerol kinase; Mttp, microsomal triglyceride transfer protein) mRNAs were measured by quantitative RT-PCR assays and normalized to the amount of 18S RNA in each sample (mean±SD; n=7). Statistically significant differences between L^{KO} and L^{WT} are indicated (*, P<0.05; **, P<0.01).

FIG. 37A and FIG. 37B are graphs of blood glucose following an ITT (mean±SD; n=10) (37A) or GTT (mean±SD, n=10˜15) (37B) performed with M^{KO} and M^{WT} male mice fed either a chow diet (ND) or a high fat diet (HFD) for 16 weeks. Statistically significant differences between M^{KO} and M^{WT} are indicated (*, P<0.05).

FIG. 37C is a graph of blood insulin concentration following administration of glucose (2 g/kg body mass) by intraperitoneal injection (mean±SD; n=13˜15). No statistically significant differences between M^{KO} and M^{WT} mice were detected (P>0.05).

FIG. 37D and FIG. 37E are bar graphs of the blood glucose concentration in chow-fed and HFD-fed (HF) M^{WT} and M^{KO} mice that were fasted overnight (D) or fed ad libitum (E) (mean±SD; n=10˜15). No statistically significant differences were detected (P>0.05).

FIGS. 37F-37L are bar graphs of the blood concentration of insulin, adipokines, and cytokines in chow-fed (ND) and HFD-fed (HF) M^{WT} and M^{KO} mice fasted overnight (mean±SD; n=10˜15). Statistically significant differences are indicated (*, P<0.05).

FIG. 38A-38F are bar graphs of insulin-stimulated whole body glucose turnover (A); whole body glycolysis (B); whole body glycogen plus lipid synthesis (C); basal hepatic glucose production (HGP) (D); insulin-stimulated rate of HGP during the clamp (E); hepatic insulin action, expressed as insulin-mediated percent suppression of basal HGP (F). The data presented are the mean±SE for 6˜9 experiments. Statistically significant differences between M^{KO} mice and M^{WT} mice are indicated (*, P<0.05).

FIG. 38G and FIG. 38H are bar graphs of glucose uptake in (G) white adipose tissue and (H) gastronemius muscle during a hyperinsulinemic-euglycemic clamp study. The data are expressed as the percent suppression of glucose uptake caused by feeding a HFD and presented as the mean±SE for 4˜9 experiments. Statistically significant differences between M^{KO} mice and M^{WT} mice are indicated (*, P<0.05).

FIGS. 39A-39C are representations of immunoblots to detect JNK1, AKT, phospho-AKT, and GAPDH in extracts prepared from (A) gastronemius muscle, (B) liver, and (C) epididymal adipose tissue at 10 mins post-injection of insulin (1.5 U/kg body mass) into chow-fed (ND) and HFD-fed (HF) M^{WT} and M^{KO} mice that were fasted overnight.

FIG. 40A contains representative sections of the liver, stained with H&E (scale bar=100 μm), from chow-fed (ND) and HFD-fed (HF) M^{WT} (left panels) and M^{KO} mice (right panels) that were fasted overnight. FIG. 40B is a bar graph of the amount of hepatic triglyceride in mice fasted overnight (mean±SD; n=10). Statistically significant differences between M^{KO} and M^{WT} are indicated (*, P<0.05; **, P<0.01).

FIG. 41 is a series of bar graphs of the relative mRNA expression of the indicated transcription factors (C/ebpα, C/ebpβ, Pparγ, and Srebp1), co-activators (Pgc1α and Pgc1β), fatty acid synthase (Fas), and microsomal triglyceride transfer protein (Mttp). The relative mRNA expression was calculated by normalization of the data to the amount of 18S RNA in each sample (mean±SD; n=8˜10). Statistically significant differences are indicated (*, P<0.05).

FIG. 42 is a series of bar graphs of the relative mRNA expression of Tnfα, I16, interferon-γ (Ifnγ), Cd68, Icam1, lysozyme (Lyzs), and cytochrome p450 2E1 (Cye2e1). The relative mRNA expression was calculated by normalization of the data to the amount of 18S RNA in each sample (mean±SD; n=8˜10). Statistically significant differences are indicated (*, P<0.05; **, P<0.01).

FIG. 43A contains bar graphs of the amount of cholesterol, triglyceride and free fatty acid (FFA) in the blood of HFD-fed M^{WT} and M^{KO} mice that were fasted overnight (mean±SD; n=10). Statistically significant differences between M^{KO} and M^{WT} are indicated (*, P<0.05).

FIG. 43B contains graphs of the FPLC profiles of cholesterol (upper panels) and triglyceride (lower panels) (Mean±SD; n=10).

FIG. 44A is a series of representations of histological sections of epididymal adipose tissue stained with H&E (left panels) and with an antibody (F4/80) to a macrophage marker (right panels) from chow-fed (ND) and HFD-fed (HF) M^{WT} and M^{KO} mice that were fasted overnight. Scale bar=100 μm.

FIG. 44B is a series of bar graphs of the relative mRNA expression of the indicated molecules in epididymal adipose tissue as measured by quantitative RT-PCR analysis. The relative mRNA expression was calculated by normalization of the data to the amount of Gapdh mRNA in each sample (mean±SD; n=8˜10). Statistically significant differences are indicated (*, P<0.05; **, P<0.01).

FIG. 45A is a representation of the genotype analysis of genomic DNA isolated from the Cortex, Cerebellum (Cerb.), Hippocampus (Hippo.), Hypothalamus (Hypoth.), and Medulla Oblongata (M. Oblong.) of N^{WT} mice and N^{KO} mice to detect the presence of Jnk1⁺, Jnk1^{LoxP} and Jnk1Δ alleles.

FIG. 45B is a representation of an immunoblot to detect JNK1 and GAPDH in extracts prepared from the Cortex, Cerebellum, Liver, quadriceps muscle, epididymal adipose tissue (White Fat), Hypothalamus, and Hippocampus of N^{KO} and N^{WT} mice.

FIG. 45C is a graph of the weight (mean±SD; n=10) of N^{KO} and N^{WT} male mice (8-10 weeks old) fed either a chow diet (ND) or a HFD (16 wks). The HFD-induced weight gain of N^{WT} was significantly greater than N^{KO} mice (P<0.05).

FIG. 45D contains representations depicting JNK activity, as measured by a protein kinase activity (KA) assay using ATP[γ-³²P] and cJun as substrates, in the liver, quadriceps muscle, and epididymal adipose tissue of N^{KO} and N^{WT} mice fed a chow diet (ND) or a high fat (HF) diet for 16 wks. The cell extracts used for the protein kinase assay were also examined by immunoblot analysis by probing with antibodies to JNK1 and GAPDH.

FIG. 46A contains bar graphs of the blood glucose concentration in chow-fed (ND) and HFD-fed N^{KO} and N^{WT} mice fasted overnight or fed ad-libitum. The blood concentration of insulin and leptin in mice fasted overnight is also presented. The data represent the mean±SD (n=10). Statistically significant differences between N^{KO} and N^{WT} are indicated (*, P<0.05).

FIG. 46B is a graph of blood glucose concentration in chow-fed (ND) and HFD-fed N^{KO} and N^{WT} mice following intraperitoneal injection of glucose (1 g/kg). The data presented represent the mean±SD (n=10-15). Statistically significant differences between N^{KO} and N^{WT} are indicated (*, P<0.05).

FIGS. 46C and 46D are graphs of the concentration of blood glucose (mean±SD; n=10) following intraperitoneal injection of insulin (1.5 U/kg body mass) to N^{KO} and N^{WT} mice fed either a chow diet (ND) or a HFD. Statistically significant differences between N^{KO} and N^{WT} are indicated (***, P<0.001).

FIG. 46E is a graph of glucose-induced insulin release. The effect of administration of glucose (2 g/kg body mass) by intraperitoneal injection on blood insulin concentration was examined (mean±SD; n=13˜15). No statistically significant differences between N^{KO} and N^{WT} mice were detected (P>0.05).

FIG. 47 is a series of bar graphs of the gas exchange (V_{O2} and V_{CO2}), respiratory exchange quotient [V_{CO2}]/[V_{O2}], energy expenditure, and physical activity of groups of 6 mice examined using metabolic cages. Statistically significant differences between N^{KO} mice and N^{WT} mice are indicated (*, P<0.05; **, P<0.01; ***, P<0.001).

FIG. 48A is a bar graph of the body temperature of chow-fed (ND) and HFD-fed N^{KO} and N^{WT} mice (mean±SD; n=8). Statistically significant differences between N^{KO} and N^{WT} mice are indicated (*, P<0.05).

FIG. 48B is a series of representations of sections prepared from intrascapular brown fat of chow-fed (ND) and HFD-fed N^{KO} and N^{WT} mice and stained with H&E. Scale bar=100 μm.

FIG. 48C is a series of bar graphs of the concentration of T3, T4, and TSH in the blood of chow-fed (ND) and HFD-fed N^{KO} and N^{WT} mice as measured by ELISA (mean±SD, n=10). FIG. 48C also contains a bar graph of the relative mRNA expression of Thrh mRNA in the hypothalamus as measured by quantitative RT-PCR analysis and normalized to the amount of Gapdh mRNA measured in each sample (Mean±SD; n=6˜7). Statistically significant differences between N^{KO} and N^{WT} mice are indicated (*, P<0.05; **, P<0.01).

FIG. 49A is a bar graph of the body weight of N^{KO} and N^{WT} mice treated with PTU in the drinking water. The mice were divided into chow-fed (ND) and HFD-fed groups after 2 wks. and then maintained for an additional 10 wks. No statistically significant differences between N^{KO} and N^{WT} mice were detected (P>0.05).

FIG. 49B is a series of bar graphs of blood glucose concentration in fed and overnight fasted mice, body temperature, and blood hormone (Insulin, Leptin, and Resistin) concentrations in chow-fed (ND) and HFD-fed N^{KO} and N^{WT} mice examined after 12 wks of treatment with PTU. No significant differences between N^{KO} and N^{WT} mice were detected (P>0.05).

FIG. 49C contains a bar graph of the glucose tolerance tests (left panel) and insulin tolerance tests (right panel) on PTU-treated chow-fed (ND) and HFD-fed N^{KO} and N^{WT} mice. No significant differences between N^{KO} and N^{WT} mice were detected (P>0.05).

Like reference symbols in the various drawings indicate like elements.

### DETAILED DESCRIPTION

In general, the present invention provides methods for monitoring JNK inhibition in vitro (e.g., in cells in culture) or in vivo (e.g., in subjects being treated with a JNK inhibitor) based on levels of IL6 or TNFα, which decrease (e.g., in a dose-dependent manner) with JNK deficiency. As such, IL6 and TNFα can serve as biomarkers of JNK inhibition. In particular, IL6 can serve as a biomarker of JNK1 inhibition in adipose tissue. Identification of biomarkers of JNK inhibition that are readily detectable and quantifiable in the serum is particularly useful as JNKs activate such a wide range of proteins. Methods of the invention can include detecting the level of IL6 or TNFα in the cells, culture media, or biological samples from the treated subjects. Levels of IL6 and TNFα also can be used to identify JNK inhibitors in vitro.

The term “JNK” as used herein refers to a family of mammalian cJun NH₂-terminal kinases (e.g., from mice or humans), including JNK1, JNK2, and JNK3, and alternative splice variants thereof. A representative nucleic acid sequence encoding human JNK1 can be found in GenBank under Accession No. L26318. A representative nucleic acid sequence encoding human JNK2 can be found in GenBank under Accession No. L31951. A representative nucleic acid sequence encoding human JNK3 can be found in GenBank under Accession No. HSU34819. JNK1 and JNK2 are present ubiquitously while JNK3 is expressed predominantly in the brain.

As described herein, JNK plays a role in the development of hepatitis. Unexpectedly, JNK appears to play no role in TNFα-stimulated death of hepatocytes, but instead, is essential for TNFα expression by hematopoietic cells, including resident inflammatory cells in the liver (e.g., Kupffer cells and natural killer T (NKT) cells), which results in the development of hepatitis. As such, treatment of hepatitis with a JNK inhibitor can be monitored by measuring levels of TNFα.

In addition, based on the results described herein, toxicity of JNK inhibitors can be reduced by selectively targeting JNK inhibitors to particular tissues, e.g., adipose tissue. Adipose tissue-derived IL6 is an important mediator of hepatic insulin resistance. JNK1 deficiency in adipose tissue suppresses high fat diet-induced expression of IL6 and suppresses insulin resistance in the liver. JNK1-dependent secretion of the inflammatory cytokine IL6 by adipose tissue causes increased expression of liver suppressor of cytokine signaling-3 (SOCS3), a protein that induces hepatic insulin resistance. In other words, JNK1 activation in adipose tissue can cause insulin resistance in the liver. Conversely, JNK1 deficiency in adipose tissue can cause increased hepatic insulin sensitivity. Loss of JNK1 in liver, however, provides no protection against obesity-induced insulin resistance. Thus, methods of the invention also can include selectively targeting JNK1 inhibitors to adipose tissue for treating type 2 diabetes and insulin resistance.

### Methods of Monitoring JNK Inhibition

JNK inhibition can be monitored in a subject (e.g., a human patient) being treated with a JNK inhibitor (e.g., a JNK inhibitor described herein) by determining the level of IL6 or TNFα in a biological sample from the subject being treated. “Determining the level of IL6 or TNFα” refers to a quantitative measurement of the amount of IL6 or TNFα in the biological sample. IL6 and TNFα can be detected and measured as described herein. In some embodiments, the amount of IL6 or TNFα protein can be determined in the biological sample. In some embodiments, the amount of mRNA encoding IL6 or TNFα can be determined in the biological sample. Suitable biological samples can include, for example, whole blood, plasma, serum, or adipose or liver tissue (e.g., from a biopsy). Serum is a particularly useful biological sample.

A decrease in the level of IL6 in the biological sample indicates a positive response to the therapy with the JNK inhibitor. The level of IL6 or TNFα in the biological sample can be compared to a control level such as the level of IL6 or TNFα in the subject before treatment with the JNK inhibitor (i.e., the pre-treatment level of IL6 or TNFα). Alternatively, the control level can be that of a control population (e.g., the average serum IL6 or TNFα level in a group of subjects with or without a particular disorder). Typically, levels of IL6 and TNFα will decrease in the biological sample dependent on the dosage of JNK inhibitor administered to the subject. For example, a large decrease in the level of IL6 or TNFα in the biological sample relative to a control level indicates a higher dosage of JNK inhibitor has been administered to the subject. Depending on the condition for which the subject is being treated, different dosages of JNK inhibitor can be required.

### Detecting IL6 or TNFα Protein

IL6 or TNFα protein can be detected, for example, immunologically using one or more antibodies. The term “antibody” as used herein refers to intact antibodies as well as antibody fragments that retain some ability to bind to an epitope. Such fragments include, without limitation, Fab, F(ab′)2, and Fv antibody fragments. The term “epitope” refers to an antigenic determinant on an antigen to which the paratope of an antibody binds. Epitopic determinants usually consist of chemically active surface groupings of molecules (e.g., amino acid or sugar residues) and usually have specific three dimensional structural characteristics as well as specific charge characteristics. Antibodies that bind to IL6 or TNFα are commercially available, e.g., from Millipore (Billerica, Mass.) or can be easily made using standard techniques.

In immunological assays, an antibody having specific binding affinity for IL6 or TNFα, or a secondary antibody that binds to such an antibody, can be labeled either directly or indirectly. Suitable labels include, without limitation, radionuclides (e.g., ¹²⁵I, ¹³¹I, ³⁵S, ³H, ³²P, ³³P, or ¹⁴C), fluorescent moieties (e.g., fluorescein, FITC, APC, PerCP, rhodamine, or PE), luminescent moieties (e.g., Qdot™ nanoparticles supplied by the Quantum Dot Corporation, Palo Alto, Calif.), compounds that absorb light of a defined wavelength, or enzymes (e.g., alkaline phosphatase or horseradish peroxidase). Antibodies can be indirectly labeled by conjugation with biotin then detected with avidin or streptavidin labeled with a molecule described above. Methods of detecting or quantifying a label depend on the nature of the label and are known in the art. Examples of detectors include, without limitation, x-ray film, radioactivity counters, scintillation counters, spectrophotometers, colorimeters, fluorometers, luminometers, and densitometers. Combinations of these approaches (including “multi-layer” assays) familiar to those in the art can be used to enhance the sensitivity of assays.

Immunological assays for detecting IL6 or TNFα can be performed in a variety of known formats, including sandwich assays, competition assays (competitive RIA), or bridge immunoassays. See, for example, U.S. Pat. Nos. 5,296,347; 4,233,402; 4,098,876; and 4,034,074. Methods of detecting IL6 or TNFα generally include contacting a biological sample (e.g., whole blood, plasma, serum, or a tissue sample) with an antibody that binds to IL6 or TNFα and detecting binding of IL6 or TNFα to the antibody. In some embodiments, the assays are done in solution. In other embodiments, a solid substrate is used. For example, an antibody having specific binding affinity for IL6 or TNFα can be immobilized on a solid substrate by any of a variety of methods known in the art and then exposed to the biological sample. Binding of IL6 or TNFα to the antibody on the solid substrate can be detected by exploiting the phenomenon of surface plasmon resonance, which results in a change in the intensity of surface plasmon resonance upon binding that can be detected qualitatively or quantitatively by an appropriate instrument, e.g., a Biacore apparatus (Biacore International AB, Rapsgatan, Sweden). Alternatively, the antibody can be labeled and detected as described above. A standard curve using known quantities of IL6 can be generated to aid in the quantitation of IL6 levels. Similarly, a standard curve using known quantities of TNFα can be generated to aid in the quantitation of TNFα levels.

In other embodiments, a “sandwich” assay in which a capture antibody is immobilized on a solid substrate is used to detect the level of IL6 or TNFα. The solid substrate can be contacted with the biological sample such that any IL6 or TNFα in the sample can bind to the immobilized antibody. The level of IL6 or TNFα bound to the antibody can be determined using a “detection” antibody having specific binding affinity for IL6 or TNFα and the methods described above. It is understood that in these sandwich assays, the capture antibody should not bind to the same epitope (or range of epitopes in the case of a polyclonal antibody) as the detection antibody. Thus, if a monoclonal antibody is used as a capture antibody, the detection antibody can be another monoclonal antibody that binds to an epitope that is either completely physically separated from or only partially overlaps with the epitope to which the capture monoclonal antibody binds, or a polyclonal antibody that binds to epitopes other than or in addition to that to which the capture monoclonal antibody binds. If a polyclonal antibody is used as a capture antibody, the detection antibody can be either a monoclonal antibody that binds to an epitope that is either completely physically separated from or partially overlaps with any of the epitopes to which the capture polyclonal antibody binds, or a polyclonal antibody that binds to epitopes other than or in addition to that to which the capture polyclonal antibody binds. Sandwich assays can be performed as sandwich ELISA assays, sandwich Western blotting assays, or sandwich immunomagnetic detection assays.

Suitable solid substrates to which an antibody (e.g., a capture antibody) can be bound include, without limitation, microtiter plates, tubes, membranes such as nylon or nitrocellulose membranes, and beads or particles (e.g., agarose, cellulose, glass, polystyrene, polyacrylamide, magnetic, or magnetizable beads or particles). Magnetic or magnetizable particles can be particularly useful when an automated immunoassay system is used. In some embodiments, beads or particles dyed with fluorophores can be coated with an antibody having specific binding affinity for IL6 or TNFα, and used to detect IL6 or TNFα from samples using the Luminex® 200 system.

Alternative techniques for detecting IL6 and TNFα include mass-spectrophotometric techniques such as electrospray ionization (ESI), and matrix-assisted laser desorption-ionization (MALDI). See, for example, Gevaert et al., Electrophoresis, 22, 1645 (2001); Chaurand et al., J. Am. Soc. Mass. Spectrom., 10, 91 (1999). Mass spectrometers useful for such applications are available from Applied Biosystems (Foster City, Calif.); Bruker Daltronics (Billerica, Mass.) and Amersham Pharmacia (Sunnyvale, Calif.).

### Detecting IL6 and TNFα Ribonucleic Acid

IL6 or TNFα RNA can be detected, for example, by polymerase chain reaction (PCR) assays or RNA blotting techniques (e.g., Northern blotting). In general, PCR refers to amplification of a target nucleic acid, using sequence information from the ends of the region of interest or beyond to design oligonucleotide primers that are identical or similar in sequence to opposite strands of the template to be amplified. Primers are typically 14 to 40 nucleotides in length, but can range from 10 nucleotides to hundreds of nucleotides in length. PCR is described, for example in PCR Primer: A Laboratory Manual, Ed. by Dieffenbach, C. and Dveksler, G., Cold Spring Harbor Laboratory Press, 1995. Nucleic acids also can be amplified by ligase chain reaction, strand displacement amplification, self-sustained sequence replication or nucleic acid sequence-based amplification. See, for example, Lewis, Genetic Engineering News, 12, 1 (1992); Guatelli et al., Proc. Natl. Acad. Sci. USA, 87, 1874 (1990); and Weiss, Science, 254, 1292 (1991). For example, the levels of IL6 or TNFα mRNA can be detected using reverse transcription-polymerase chain reaction (RT-PCR). Real-time quantitative PCR can be performed using, for example, the ABI PRISM 7700 Sequence Detection System and Taqman fluorogenic probes, or the LightCycler™ instrument from Roche.

### Treating and Monitoring Therapy

In some embodiments, a JNK inhibitor (e.g., JNK1 inhibitor) is administered to a subject such as a human patient that has been diagnosed with metabolic syndrome (i.e., a group of disorders that result in an increased risk of coronary heart disease, stroke, peripheral vascular disease, type 2 diabetes, and includes symptoms such as insulin resistance, glucose intolerance, dyslipidemia, and hypertension). JNK inhibitors also can be administered prophylactically in patients at risk for developing metabolic syndrome to inhibit or hinder the development of symptoms of the disease from occurring, delay onset of symptoms, or lessen the severity of subsequently developed disease symptoms. In either case, an amount of a JNK inhibitor effective to treat the metabolic syndrome is administered to the patient. Treatment of metabolic syndrome can include reducing the severity of the disease or slowing progression of the disease. For example, a JNK inhibitor can reduce insulin resistance in the liver.

In some embodiments, a JNK inhibitor (e.g., JNK1 inhibitor) is administered to a mammal such as a human patient that has been diagnosed with an inflammatory condition (e.g., rheumatoid arthritis, encephalitis, psoriasis, inflammatory bowel disease (IBD), hepatitis, asthma, or organ transplant) or liver damage. JNK inhibitors also can be administered prophylactically in patients at risk for developing inflammatory conditions to inhibit or hinder the development of symptoms of the disease from occurring, delay onset of symptoms, or lessen the severity of subsequently developed disease symptoms. In either case, an amount of a JNK inhibitor effective to treat the inflammatory condition is administered to the patient. Treatment of an inflammatory condition can include reducing the severity of the disease or slowing progression of the disease.

As used herein, the term “effective amount” refers to an amount of a JNK inhibitor that reduces the deleterious effects of the metabolic syndrome or inflammatory condition without inducing significant toxicity to the host. Effective amounts of JNK inhibitors can be determined by a physician, taking into account various factors that can modify the action of drugs such as overall health status, body weight, sex, diet, time and route of administration, other medications, and any other relevant clinical factors.

Methods described herein can include monitoring IL6 or TNFα levels in the subject such that the dosage of the JNK inhibitor can be tailored to the subject's response to the inhibitor. For example, the dosage of the JNK inhibitor can be increased or decreased based on the level of IL6 or TNFα in a biological sample from the subject. For example, if IL6 or TNFα levels of a treated subject do not decrease after administration of a JNK inhibitor, the dosage of the JNK inhibitor can be increased. Alternatively, if IL6 levels of a treated subject have decreased and the subject is improving (e.g., insulin resistance is decreased or insulin sensitivity is increased), the dosage of JNK inhibitor can be reduced. If TNFα levels of a treated subject have decreased and the subject is improving (e.g., increased liver function), the dosage of JNK inhibitor can be reduced. IL6 and TNFα levels can be determined as described herein.

Typically, IL6 and TNFα levels are compared to a control level. For example, the control level can be the pre-treatment level of IL6 or TNFα. Alternatively, the control level can be that of a control population (e.g., the average serum IL6 or TNFα level in a group of subjects with or without a particular disorder).

Methods described herein can include monitoring the metabolic syndrome or inflammatory condition to, for example, determine if the condition is improving with treatment. Any method can be used to monitor metabolic syndrome or an inflammatory condition. For example, insulin resistance or glucose intolerance can be monitored in a subject with metabolic syndrome. For an inflammatory condition such as hepatitis, alanine transaminase (ALT) and/or aspartate aminotrasferase (AST) activity can be monitored in serum to assess liver function.

Suitable inhibitors can decrease the expression of a nucleic acid encoding JNK, decrease levels of the JNK protein, or inhibit JNK activity. JNK inhibitors that can be used include, for example, peptide inhibitors such as XG-102 (D-JNK1); aryl-oxindole compounds (e.g., 4,5-pyridazinoxindoles from Hoffmann-LaRoche); benzazole compounds (e.g., pyrimidinyl-substituted benzazole-acetonitriles from Merck Serono); anthrapyrazolone compounds (e.g., SP600125 (anthra[1,9]pyrazol-6(2H)-one), CC-401, or CC-930 from Celgene Corp., Summit, N.J.); sulfonyl amino acid, sulfonamide, and sulfonyl hydrazide compounds (e.g., from Merck Serono); 3-oximido-oxindole analogues (e.g., from Vertex); 4-substituted isoxazole analogues containing a 2-anilinopyridine or 2-anilinopyrmidine moiety; azoles; 4-aryl- or 4-alykynyl-isoindolones (e.g., from Hoffmann-LaRoche); tetrahydropyrimidine; or other heterocyclic compounds, and pharmaceutically acceptable salts thereof. See, for example, U.S. Patent Publication No. 20070270448; U.S. Pat. No. 6,410,693; WO 00/35909; WO 00/35906; WO 01/47920; WO 01/12609; WO 00/75118; WO 01/12621; Borsello et al., Nat Med 9, 1180 (2003); Manning and Davis, 2003, supra; and Borgoyevitch and Arthur, 2008, supra.

A number of nucleic acid based methods, including antisense, ribozyme directed RNA cleavage, and post-transcriptional gene silencing (PTGS), e.g., double-stranded small interfering RNA (siRNA), also can be used to reduce expression of JNK. Alternatively, such nucleic acid based methods can be used to target IL6. Antisense oligonucleotides typically are at least 8 nucleotides in length. For example, an antisense oligonucleotide can be about 8, 9, 10-20 (e.g., 11, 12, 13, 14, 15, 16, 17, 18, 19, or 20 nucleotides in length), 15 to 20, 18-25, or 20-50 nucleotides in length. In other embodiments, antisense molecules can be used that are greater than 50 nucleotides in length, including the full-length sequence of a JNK mRNA.

As used herein, the term “oligonucleotide” refers to an oligomer or polymer of ribonucleic acid (RNA) or deoxyribonucleic acid (DNA) or analogs thereof. Nucleic acid analogs can be modified at the base moiety, sugar moiety, or phosphate backbone to improve, for example, stability, hybridization, or solubility of a nucleic acid. Modifications at the base moiety include substitution of deoxyuridine for deoxythymidine, and 5-methyl-2′-deoxycytidine and 5-bromo-2′-deoxycytidine for deoxycytidine. Other examples of nucleobases that can be substituted for a natural base include 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Other useful nucleobases include those disclosed, for example, in U.S. Pat. No. 3,687,808.

Modifications of the sugar moiety can include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six-membered, morpholino ring, or peptide nucleic acids, in which the deoxyphosphate backbone is replaced by a pseudopeptide backbone (e.g., an aminoethylglycine backbone) and the four bases are retained. See, for example, Summerton and Weller (1997) Antisense Nucleic Acid Drug Dev., 7:187-195; and Hyrup et al. (1996) Bioorgan. Med. Chem., 4:5-23. In addition, the deoxyphosphate backbone can be replaced with, for example, a phosphorothioate or phosphorodithioate backbone, a phosphoroamidite, or an alkyl phosphotriester backbone. See, for example, U.S. Pat. Nos. 4,469,863, 5,235,033, 5,750,666, and 5,596,086 for methods of preparing oligonucleotides with modified backbones.

Antisense oligonucleotides also can be modified by chemical linkage to one or more moieties or conjugates that enhance the activity, cellular distribution or cellular uptake of the oligonucleotide. Such moieties include but are not limited to lipid moieties (e.g., a cholesterol moiety); cholic acid; a thioether moiety (e.g., hexyl-5-tritylthiol); a thiocholesterol moiety; an aliphatic chain (e.g., dodecandiol or undecyl residues); a phospholipid moiety (e.g., di-hexadecyl-rac-glycerol or triethyl-ammonium 1,2-di-O-hexadecyl-rac-glycero-3-H-phosphonate); a polyamine or a polyethylene glycol chain; adamantane acetic acid; a palmityl moiety; or an octadecylamine or hexylaminocarbonyl-oxycholesterol moiety. The preparation of such oligonucleotide conjugates is disclosed in, for example, U.S. Pat. Nos. 5,218,105 and 5,214,136.

Methods for synthesizing antisense oligonucleotides are known, including solid phase synthesis techniques. Equipment for such synthesis is commercially available from several vendors including, for example, Applied Biosystems (Foster City, Calif.). Alternatively, expression vectors that contain a regulatory element that directs production of an antisense transcript can be used to produce antisense molecules.

Antisense oligonucleotides can bind to a nucleic acid encoding JNK, including DNA encoding JNK RNA (including pre-mRNA and mRNA) transcribed from such DNA, and also cDNA derived from such RNA, under physiological conditions (i.e., physiological pH and ionic strength). It is understood in the art that the sequence of an antisense oligonucleotide need not be 100% complementary to that of its target nucleic acid to be hybridizable under physiological conditions. Antisense oligonucleotides hybridize under physiological conditions when binding of the oligonucleotide to the JNK1 nucleic acid interferes with the normal function of the JNK nucleic acid, and non-specific binding to non-target sequences is minimal.

Target sites for JNK antisense oligonucleotides include the regions encompassing the translation initiation or termination codon of the open reading frame (ORF) of the gene. In addition, the ORF has been targeted effectively in antisense technology, as have the 5′ and 3′ untranslated regions. Furthermore, antisense oligonucleotides have been successfully directed at intron regions and intron-exon junction regions. For example, the following antisense oligonucleotides can be used to reduce expression of JNK1: 5′-CTCTCTGTAGGCCCGCTTGG-3′ (SEQ ID NO:3) or 5′-CTCATGATGGCAAGCAATTA-3′ (SEQ ID NO:4). See, for example, Tafolla et al., J. Biol. Chem., 280, 19992 (2005). Further criteria can be applied to the design of antisense oligonucleotides. Such criteria are well known in the art, and are widely used, for example, in the design of oligonucleotide primers. These criteria include the lack of predicted secondary structure of a potential antisense oligonucleotide, an appropriate G and C nucleotide content (e.g., approximately 50%), and the absence of sequence motifs such as single nucleotide repeats (e.g., GGGG runs). The effectiveness of antisense oligonucleotides at modulating expression of a JNK nucleic acid can be evaluated by measuring levels of the JNK mRNA or protein (e.g., by Northern blotting, RT-PCR, Western blotting, ELISA, or immunohistochemical staining).

In another method, a ribozyme or catalytic RNA can be used to affect expression of an mRNA, such as a JNK1 mRNA. See, U.S. Pat. No. 6,423,885. Ribozymes can be designed to specifically pair with virtually any target RNA and cleave the phosphodiester backbone at a specific location, thereby functionally inactivating the target RNA. Heterologous nucleic acids can encode ribozymes designed to cleave particular mRNA transcripts, thus preventing expression of a polypeptide. Hammerhead ribozymes are useful for destroying particular mRNAs, although various ribozymes that cleave mRNA at site-specific recognition sequences can be used. Hammerhead ribozymes cleave mRNAs at locations dictated by flanking regions that form complementary base pairs with the target mRNA. The sole requirement is that the target RNA contains a 5′-UG-3′ nucleotide sequence. The construction and production of hammerhead ribozymes is known in the art. See, for example, U.S. Pat. No. 5,254,678 and WO 02/46449 and references cited therein. Hammerhead ribozyme sequences can be embedded in a stable RNA such as a transfer RNA (tRNA) to increase cleavage efficiency in vivo. See, for example, Perriman et al., Proc. Natl. Acad. Sci. USA, 92, 6175 (1995). RNA endoribonucleases such as the one that occurs naturally in Tetrahymena thermophila also can be useful. See, for example, U.S. Pat. Nos. 4,987,071 and 6,423,885.

In another embodiment, PNA (polyamide nucleic acid or peptide nucleic acid) oligomers can be used to reduce expression of JNK in a subject. PNA oligomers are modified oligonucleotides in which the phosphodiester backbone of the oligonucleotide is replaced with a neutral polyamide backbone consisting of N-(2-aminoethyl)glycine units linked through amide bonds. See, e.g., Nielsen et al., Science, 254, 1497 (1991), and Nielsen et al., Bioconjugate Chem., 5, 3 (1994).

Nucleic acid that induces RNA interference against nucleic acid encoding a JNK polypeptide also can be used to reduce expression of JNK in a subject. For example, double-stranded small interfering RNA (siRNA) homologous to a JNK1 DNA can be used to reduce expression of that DNA. One example of an siRNA oligonucleotide that can be used to reduce expression of JNK1 is the following: 5′-CGUGGAUUUAUGGUCUGUGTT-3′/3′-TTGCACCUAAAUACCAGACAC-5′ (SEQ ID NO:5). See, Dai et al., Oncogene 22, 7108 (2003). Constructs for siRNA can be constructed as described, for example, in Fire et al., Nature, 391, 806 (1998); Romano and Masino, Mol. Microbiol., 6, 3343 (1992); Cogoni et al., EMBO J., 15, 3153 (1996); Cogoni and Masino, Nature, 399, 166 (1999); Misquitta and Paterson, Proc. Natl. Acad. Sci. USA, 96, 1451 (1999); and Kennerdell and Carthew, Cell, 95, 1017 (1998). In some embodiments, a small hairpin RNA (shRNA) can be used. shRNA refers to an siRNA composed of a single strand of RNA that possesses regions of self-complementarity that cause the single strand to fold back upon itself and form a hairpin-like structure with an intramolecular duplexed region containing at least 19 basepairs. shRNAs can be readily expressed from single expression cassettes.

The sense and anti-sense RNA strands of siRNA can be individually constructed using chemical synthesis and enzymatic ligation reactions using procedures known in the art. For example, each strand can be chemically synthesized using naturally occurring nucleotides or variously modified nucleotides designed to increase the biological stability of the molecule or to increase the physical stability of the duplex formed between the sense and anti-sense strands, e.g., phosphorothioate derivatives and acridine substituted nucleotides. The sense or anti-sense strand can also be produced biologically using an expression vector into which a target sequence (full-length or a fragment) has been subcloned in a sense or anti-sense orientation. The sense and anti-sense RNA strands can be annealed in vitro before delivery of the dsRNA to cells. Alternatively, annealing can occur in vivo after the sense and anti-sense strands are sequentially delivered to neural cells.

JNK inhibitors can be administered by any route, including, without limitation, oral or parenteral routes of administration such as intravenous, intramuscular, intraperitoneal, subcutaneous, intrathecal, intraarterial, nasal, transdermal (e.g., as a patch), or pulmonary absorption. A JNK inhibitor can be formulated as, for example, a solution, suspension, or emulsion with pharmaceutically acceptable carriers or excipients suitable for the particular route of administration, including sterile aqueous or non-aqueous carriers. Aqueous carriers include, without limitation, water, alcohol, saline, and buffered solutions. Examples of non-aqueous carriers include, without limitation, propylene glycol, polyethylene glycol, vegetable oils, and injectable organic esters. Preservatives, flavorings, sugars, and other additives such as antimicrobials, antioxidants, chelating agents, inert gases, and the like also may be present.

For oral administration, tablets or capsules can be prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g. magnesium stearate, talc or silica); disintegrants (e.g., potato starch or sodium starch glycolate); or wetting agents (e.g., sodium lauryl sulfate). Tablets can be coated by methods known in the art. Preparations for oral administration can also be formulated to give controlled release of the compound.

Nasal preparations can be presented in a liquid form or as a dry product. Nebulised aqueous suspensions or solutions can include carriers or excipients to adjust pH and/or tonicity.

In some embodiments, toxicity of JNK inhibitors can be reduced by selectively targeting the inhibitor to a particular tissue (e.g., adipose tissue, Kupffer cells, or NKT cells). JNK inhibitors (e.g., a JNK inhibitor described herein) can be targeted to a particular tissue by any method. For example, in one embodiment, a JNK inhibitor can be locally delivered to one or more adipose depots. In other embodiments, JNK inhibitors can be conjugated (e.g., via a linker) to a monoclonal antibody having binding affinity for an epitope on an adipocyte (e.g., adiponurin). Monoclonal antibodies having binding affinity for adiponurin are commercially available from, for example, Abcam (Cambridge, Mass.). In other embodiments, JNK inhibitors can be conjugated to a monoclonal antibody having binding affinity for an epitope on an NKT cell (e.g., CD1) or Kupffer cell (e.g., F4/80). In other embodiments, JNK inhibitors can be conjugated to a homing protein. JNK inhibitors can be conjugated to proteins, including monoclonal antibodies, using conventional techniques and readily available linkers. JNK inhibitors also can be loaded in immunoliposomes (e.g., pegylated immunoliposomes) for selective delivery to a tissue. Alternatively, for siRNA or antisense oligonucleotides targeting JNK (e.g., JNK1 or a splice variant thereof) or IL6, the molecules can be selectively expressed in adipose tissue using a tissue-specific promoter such as a fatty acid binding protein-4 promoter or adiponectin promoter. To selectively express in Kupffer cells or NKT cells, a tissue-specific promoter such as a CD1 or F4/80 promoter can be used.

### Identifying JNK Inhibitors

JNK inhibitors can be identified using the methods described herein. For example, in vitro assays, cell based models, or in vivo models can be used to identify inhibitors of JNK (e.g., JNK1) by monitoring expression of IL6 or TNFα. Such methods can be used in a high throughput manner. Any source of small molecules, peptides, proteins, or nucleic acids can be screened using the methods described herein. For example, any large library of chemical compounds, including libraries of natural products, libraries of synthetic compounds, or diversity oriented libraries, can be screened using the methods described herein. In some embodiments, JNK inhibitors can be identified in an in vitro assay in which adipocytes (e.g., in a culture medium) are contacted with a compound and expression of IL6 is measured in the presence and absence of the compound. Adipocytes can be obtained by differentiating stem cells using one or more of dexamethasone, isobutyl methylxanthine, indomethacin, insulin, and thiazolidinedione (e.g., Gimble et al., Circ. Res., 100, 1249 (2007)) or from a cell line. Decreased expression of IL6, as measured by mRNA encoding IL6 or IL6 protein levels, indicates the compound is a JNK inhibitor. As described herein, loss of JNK1 in muscle does not prevent obesity-induced expression of IL6 by adipose tissue and instead, IL6 expression is increased. This further validates obesity-induced IL6 expression as a biomarker for JNK inhibition (e.g. caused by a drug).

In some embodiments, JNK inhibitors can be identified in an in vitro assay in which Kupffer cells and/or NKT cells in a culture medium are contacted with a compound and expression of TNFα is measured in the presence and absence of the compound. Decreased expression of TNFα, as measured by mRNA encoding TNFα or TNFα protein levels, indicates the compound is a JNK inhibitor.

After identifying a compound as a JNK inhibitor, the toxicity profile and therapeutic efficacy of the compound can be determined by standard pharmaceutical procedures in cell culture models or animal models, including dose response curves and LD₅₀ determination.

### Animal Models

The invention also features transgenic non-human mammals, the nucleated cells of which contain a disrupted Jnk1 and/or Jnk2 nucleic acid. Non-human mammals include, for example, rodents such as rats, guinea pigs, and mice, and farm animals such as pigs, sheep, goats, horses, and cattle. As used herein, “transgenic non-human mammal” includes founder transgenic non-human mammals as well as progeny of the founders, progeny of the progeny, and so forth, provided that the progeny retain the disrupted Jnk1 and/or Jnk2 nucleic acid. For example, a transgenic founder animal can be used to breed additional animals that contain a disrupted Jnk1 nucleic acid. Transgenic mice are particularly useful.

Tissues obtained from the transgenic non-human mammals (e.g., transgenic mice) and cells derived from the transgenic non-human mammals (e.g., transgenic mice) also are provided herein. As used herein, “derived from” indicates that the cells can be isolated directly from the animal or can be progeny of such cells. For example, brain, lung, liver, pancreas, adipose, heart and heart valves, muscle, kidney, thyroid, corneal, skin, blood vessels or other connective tissue can be obtained from a transgenic non-human mammal (e.g., transgenic mice).

As used herein, “disrupted Jnk1 nucleic acid” refers to a modification in the Jnk1 and/or Jnk2 nucleic acid such that the expression of functional JNK1 and/or JNK2 polypeptide is reduced or prevented. Modifications that can result in a disrupted nucleic acid include, without limitation, insertions, deletions, substitutions, and combinations thereof. Modifications can be made in any region of a Jnk1 or Jnk2 nucleic acid, including, introns, exons, promoter, or 5′- or 3′-untranslated regions. For example, a Jnk1 or Jnk2 nucleic acid can include a substitution within one or more exons, resulting in a disruption of JNK1 expression. In some embodiments, disruptions in the Jnk1 or Jnk2 nucleic acid are conditional, as described below. Typically, homologous recombination is used to disrupt an endogenous Jnk1 or Jnk2 nucleic acid in a transgenic non-human mammal. See, Shastry, Mol. Cell. Biochem., 181, 163 (1998), for a review of gene targeting technology. As such, nucleic acid constructs amenable to genomic integration by homologous recombination typically are used to disrupt a Jnk1 or Jnk2 nucleic acid. Suitable vectors for genomic integration by homologous recombination include, without limitation, pKO Scrambler, pMC1neo, and pMC1-hsv-tk, all from Stratagene (La Jolla, Calif.).

Generally, a nucleic acid construct used to produce a transgenic non-human mammal includes a nucleic acid sequence encoding a selectable marker, which is used to interrupt the targeted exon site by homologous recombination. Typically, the selectable marker is flanked by sequences homologous to the sequences flanking the desired insertion site. It is not necessary for the flanking sequences to be immediately adjacent to the desired insertion site. Suitable markers for positive drug selection include, for example, the aminoglycoside 3′ phosphotransferase gene that imparts resistance to geneticin (G418, an aminoglycoside antibiotic) or neomycin, and other antibiotic resistance markers, such as the hygromycin-B-phosphotransferase gene that imparts hygromycin resistance. Other selection systems include negative-selection markers such as the thymidine kinase (TK) gene from herpes simplex virus. Nucleic acid constructs utilizing both positive and negative drug selection also can be used. For example, a construct can contain the aminoglycoside phosphotransferase gene and the TK gene. In this system, cells are selected that are resistant to G418 and sensitive to gancyclovir.

For conditional disruptions in a Jnk nucleic acid, the nucleic acid construct also can include recognition sequences for a recombinase (e.g., Cre or Flp) flanking one or more exons of the Jnk (e.g., Jnk1) nucleic acid. For example, one or more exons can be flanked by loxP recognition sites (34 by recognition sites recognized by the Cre recombinase) or FRT recognition sites. See, Orban, et al., Proc. Natl. Acad. Sci. (1992) 89 (15): 6861-6865, for a review of Cre/lox technology, and Brand and Dymecki, Dev. Cell (2004) 6(1):7-28. In the presence of Cre recombinase, the region between the loxP recognition sites is excised.

Transgenic non-human mammals lacking JNK1 in a particular tissue (e.g., adipose tissue, liver tissue, muscle tissue, or nervous system tissue) or JNK1 and JNK2 in a particular tissue (e.g., liver tissue) can be generated by crossing animals containing a conditional Jnk1 nucleic acid and animals containing tissue-specific expression of a recombinase, and selecting animals exhibiting the desired deletion. To produce transgenic animals with tissue-specific expression of a Cre or Flp recombinase, a nucleic acid construct can be used in which the nucleic acid sequence encoding the recombinase is operably linked to a regulatory region such as a promoter that has tissue specificity. As used herein, “operably linked” refers to positioning of a regulatory region relative to a nucleic acid sequence in such a way as to permit or facilitate transcription of the target nucleic acid. For example, for tissue-specific expression in adipose tissue, the fatty acid binding protein 4 (Fabp4) promoter can be used. For tissue-specific expression in hepatocytes, the albumin promoter can be used.

The tissue-specific promoter can be responsive to a particular stimulus. An example of an inducible promoter is the tetracycline (tet)-on promoter system, which can be used to regulate transcription of the nucleic acid. In this system, a mutated Tet repressor (TetR) is fused to the activation domain of herpes simplex VP 16 (transactivator protein) to create a tetracycline-controlled transcriptional activator (tTA), which is regulated by tet or doxycycline (dox). In the absence of antibiotic, transcription is minimal, while in the presence of tet or dox, transcription is induced. Alternative inducible systems include the ecdysone or rapamycin systems. Ecdysone is an insect molting hormone whose production is controlled by a heterodimer of the ecdysone receptor and the product of the ultraspiracle gene (USP). Expression is induced by treatment with ecdysone or an analog of ecdysone such as muristerone A.

The Mx1 promoter is another example of an inducible promoter. This promoter can be induced to high levels of transcription by administrating interferon alpha, interferon beta, or synthetic double-stranded RNA (such as poly I:C). When combined with a mutant carrying a gene that has been flanked by loxP recognition sites, the expression of Cre recombinase causes the flanked gene to be removed. Mx1-Cre transgenic mice are available from The Jackson Laboratory.

In some embodiments, the nucleic acid sequence encoding the recombinase can be operably linked to a hormone binding domain such that a fusion polypeptide is produced. For example, the nucleic acid sequence encoding the recombinase can be operably linked to a nucleic acid encoding a mutated estrogen receptor ligand binding domain such that a recombinase-mutated estrogen receptor ligand binding domain fusion polypeptide is produced. The mutated estrogen receptor ligand binding domain selectively binds the estrogen analog 4-hydroxytamoxifen (4HT) and is inactive in the absence of 4HT. As such, activity of the recombinase can be induced by 4HT. By using a tissue specific promoter and the estrogen receptor ligand binding domain, tissue specificity and inducibility of the JNK1 disruption can be achieved.

Various techniques known in the art can be used to introduce nucleic acid constructs into non-human animals to produce founder lines, in which the nucleic acid construct is integrated into the genome. Such techniques include, without limitation, pronuclear microinjection (U.S. Pat. No. 4,873,191), retrovirus mediated gene transfer into germ lines (Van der Putten et al., Proc. Natl. Acad. Sci. USA, 82, 6148 (1985)), gene targeting into embryonic stem cells (Thompson et al., Cell, 56, 313 (1989)), electroporation of embryos (Lo, Mol. Cell. Biol., 3, 1803 (1983)), sperm mediated gene transfer (Lavitrano et al., Proc. Natl. Acad. Sci. USA, 99, 14230 (2002); Lavitrano et al., Reprod. Fert. Develop., 18, 19 (2006)), and in vitro transformation of somatic cells, such as cumulus or mammary cells, or adult, fetal, or embryonic stem cells, followed by nuclear transplantation (Wilmut et al., Nature, 385, 810 (1997); and Wakayama et al., Nature, 394, 369 (1998)). Pronuclear microinjection, sperm mediated gene transfer, and somatic cell nuclear transfer are particularly useful techniques.

Once transgenic animal have been generated, expression of a target nucleic acid can be assessed using standard techniques. Initial screening can be accomplished by Southern blot analysis to determine whether or not integration of the construct has taken place. For a description of Southern analysis, see sections 9.37-9.52 of Sambrook et al., 1989, Molecular Cloning, A Laboratory Manual, second edition, Cold Spring Harbor Press, Plainview; NY. PCR techniques also can be used in the initial screening.

### Articles of Manufacture

Antibodies having specific binding affinity for IL6 or TNFα can be combined with packaging material and sold as a kit for detecting IL6 or TNFα from biological samples, monitoring JNK inhibition, monitoring treatment of metabolic syndrome or an inflammatory condition, adjusting dosage of JNK inhibitors, or identifying JNK inhibitors. JNK inhibitors (e.g., JNK1 inhibitors) targeted to a particular tissue (e.g., adipose tissue) also can be combined with packaging material and sold as a kit for treating diabetes or insulin resistance. For example, a JNK1 inhibitor conjugated to an antibody specific to an antigen expressed by adipose cells can be sold, optionally with packaging material, for treating diabetes.

Components and methods for producing articles of manufactures are well known. In some embodiments, the articles of manufacture can include one or more different anti-IL6 or anti-TNFα antibodies or fragments thereof. In addition, the articles of manufacture further can include reagents such as secondary antibodies, buffers, indicator molecules, solid phases (e.g., beads), additional agents for treating metabolic syndrome, finger pricking devices, and/or other useful reagents for detecting IL6 or TNFα from biological samples, monitoring JNK inhibition, treating inflammatory conditions, or treating diabetes or insulin resistance. The antibodies or targeted inhibitors can be in a container, such as a plastic, polyethylene, polypropylene, ethylene, or propylene vessel.

In some embodiments, an anti-IL6 antibody or anti-TNFα can be included on a solid phase and incorporated into a handheld device for bedside testing. Reagents for measuring levels of other polypeptides can be included in separate containers or can be included on a solid phase with the anti-IL6 or anti-TNFα antibody. In some embodiments, a handheld device for bedside testing includes anti-IL6 antibody and one or more antibodies having specific binding affinity for other markers of metabolic syndrome (e.g., TNFα, interferon gamma (IFNγ), or macrophage migration inhibiting factor-1 (MIF-1). In some embodiments, a handheld device for bedside testing includes an anti-TNFα antibody and one or more antibodies having specific binding affinity for markers of inflammation (e.g., IFNγ, interleukin 2 (IL2), interleukin 4 (IL4), or IL6). Instructions describing how the various reagents can be used also may be included in such kits.

The invention will be further described in the following examples, which do not limit the scope of the invention described in the claims.

### EXAMPLES

### Example 1

### Methods and Materials

Mice: Jnk1^{-/-} mice have been described by Dong et al., Science 282, 2092 (1998). Jnk2^{-/-} mice have been described by Yang et al., Immunity, 9, 575 (1998). Jnk1^{f/f} mice have been described by Das et al., Proc. Natl. Acad. Sci. USA, 104, 15759 (2007). Rip-CreESR mice have been described by Dor et al., Nature 429:41-46 (2004). Fabp4-Cre mice (He et al., Proc. Natl. Acad. Sci. USA, 100, 15712 (2003)), Lyzs-Cre mice (Clausen et al., Transgenic Res., 8, 265 (1999)), Alb-Cre mice (Postic et al., J. Biol. Chem., 274, 305 (1999)), Mx1-Cre mice (Kuhn et al., Science, 269, 1427 (1995)), Nes-Cre mice (Tronche et al., Nat. Genet. 23: 99-103. 1999), B6.SJL mice, and C57BL/6J were obtained from The Jackson Laboratory. The mice were backcrossed to the C57BL/6J strain (Jackson Laboratories) and were housed in facilities accredited by the American Association for Laboratory Animal Care (AALAC). The mice were genotyped by PCR analysis of genomic DNA as described by Das et al., 2007, supra. All studies were performed using male mice.

Radiation chimeras were generated by exposure of recipient mice (e.g., congenic C57BL/6J and B6.SJL mice) to two doses of ionizing radiation (525 Gy) and reconstitution of the mice with 10⁷ donor bone marrow cells by injection into the tail vein. In some experiments, lethally irradiated C57BL/6J mice were transplanted with bone marrow derived from wild-type (hWT) or Jnk1^{-/-} (hJnk1^{-/-}) mice. These mice were maintained for 5 wks to enable the reconstitution of the hematopoietic compartment in the recipient mice with cells derived from the donor mice. The mice were then fed a standard chow (ND) diet or a high fat (HF) diet for 16 wk.

For the hepatitis studies, the JNK knockout mice were maintained on a C57BL/6J mouse strain background (back-crossed 10 generations). Deletion of floxed alleles in Mx1-Cre mice was performed by treatment of 4 week old mice with 20 μg/g polyinosinic-polycytidylic acid (polyIC) (Mikkola et al., Nature, 421, 547 (2003)) followed by recovery (4 weeks). Genomic DNA was examined using PCR amplimers (5′-CTCAGGAAGAAAGGGCTTATTTC-3′ and 5′-GAACCACTGTTCCAATTTCCATCC-3′, SEQ ID NO:19 and SEQ ID NO:20, respectively) to distinguish between the wild-type (Jnk1⁺), floxed (Jnk1^{f}), and deleted (Jnk1^{Δ}) alleles (Das et al., 2007, supra).

Three models of murine hepatitis were examined using intravenous injection of: a) 25 mg/kg ConA (Sigma); b) 35 μg/kg E. coli 0111:B lipopolysaccharide (LPS) (Sigma) plus 1 g/kg N-acetyl-galactosamine (GalN) (Sigma); and c) 20 μg/kg TNFα (R&D Systems) plus 1 g/kg GalN.

All studies with the selective ablation of the Jnk1 gene in the mouse nervous system were performed using male mice (8-24 wks old). The mice were treated with PTU in the drinking water (cherry-flavored Cool-Aid supplemented without or with 1.2 mM PTU (Sigma)). Body temperature was measured using a Microtherma 2 Type “T” Thermometer (Braintree Scientic Inc). Rip-CreESR mice were treated with 1 mg 4-hydroxytamoxifen (Sigma) by intraperitoneal injection once each day for 5 consecutive days.

Animal studies were approved by the Institutional Animal Care and Use Committee (IACUC) of the University of Massachusetts Medical School and/or Pennsylvania State University College of Medicine.

Tissue culture: Primary bone marrow-derived macrophages were prepared and cultured using methods described by Kim et al., J. Immunol., 172, 3003 (2004). Primary bone marrow-derived macrophages were prepared and cultured using methods described by Kim et al., J. Immunol., 172, 3003 (2004). Primary CD4⁺ T cells from lymph nodes and spleen were isolated by positive selection using anti-CD4 MACS beads (Miltenyi) and cultured in vitro. Murine pancreatic islets were isolated using methods described by Mangada et al., Diabetes 58: 165-173 (2009).

RNA analysis: The expression of mRNA was examined by quantitative PCR analysis using a 7500 Fast Real Time PCR machine (Applied Biosystems). Taqman® assays were used to quantitate adiponectin (Mm00456425_m1), Cd68 (Mm00839636_g1), Fabp4 (Mm00445880_m1), Glucose 6 kinase (Mm00439129_m1), G6P (Glucose 6 phosphatase; Mm00839363_m1), IL6 (Mm00446190_m1), IL13 (Mm00434204_m1), Jnk1 (Mm0048915_m1), Lysozyme (Mm00727183_m1), Rbp4 (Mm00803266_m1), Steap4 (Mm004754022_m1), Pbef1 (Mm00451938_m1), Pepck (Mm00440636_m1), TNFα (Mm00443258_m1), Bax (Mm00432050_m1), cJun (Mm 00495062_m1), cFos (Mm00487425_m1), JunB (Mm00492781_s1), JunD (Mm00495088_s1), IL22 (Mm00444241_m1), p53 (Mm00441964_g1), p21 (Mm00432448_m1), Mdm2 (Mm00487656_m1), Puma (Mm00519268_m1), Acacα (Mm01304289_m1), Acacβ (Mm01204683_m1), Acot3 (Mm00652967_m1), Acsl1 (Mm00484217_m1), Acs14 (Mm00490331_m1), Ceacam1 (Mm00442360_m1), Cyp2e1 (Mm00491127_m1), Dgat1 (Mm00515643_m1), Fas (Mm00662322_g1), Foxo1 (Mm0049072_m1), Gyk (Mm00433896_m1), Hnf4α (Mm00433964_m1), Icam1 (Mm00516023_m1), InsR (Mm00439693_m1), Lysozyme (Mm00727183_m1), Mttp (Mm00435015_m1), Pepck (Mm00440636_m1), Pgc1α (Mm00447183_m1), C/ebpα (Mm00514283_s1), C/ebpβ (Mm00843434_s1), Ifn-γ (Mm00801778_m1), Lpl (Mm00434770_m1), Pgc1α (Mm00447183_m1), Scd1 (Mm00772290_m1), Accβ (Mm01204683_m1), Glut4 (Mm00436615_m1), Ldhβ (Mm00493146_m1), Spot14 (Mm01273967_m1), Trh (Mm01963590_s1), Tshα (Mm00617505_m1), or Ucp1 (Mm01244861-m1) with probes purchased from Applied Biosystems (Applied Biosystems). Amplimers for Pparγ (TGTGGGGATAAAGCATCAGGC and CCGGCAGTTAAGATCACACCTA, SEQ ID NOs:6 and 7, respectively), Mcp-1 (Tuncman et al., Proc. Natl. Acad. Sci. USA, 103, 10741 (2006)), and Mif (Tuncman et al. 2006, supra) were employed using Sybr Green detection. The amount of Pgc1β mRNA (TACATGCATACCTACTGCCTGCCT & TTGGGCCAGAAGTTCCCTTAGGAT, SEQ ID NOs:8 and 9, respectively), Srebp1 mRNA (GATGTGCGAACTGGACACCAG & CATAGGGGGCGTCAAACAG, SEQ ID NOs:10 and 11, respectively), IL10 mRNA (CTGGACAACATACTGCTAACCG & GGGCATCACTTCTACCAGGTAA, SEQ ID NOs:12 and 13, respectively), IL12 mRNA (CCATTTTCCTTCTTGTGGAGCA & AGACATGGAGTCATAGGCTCTG, SEQ ID NOs:14 and 15, respectively), and Tgfβ1 mRNA (TGGTTTGCCATCGTTTTGCTG & ACAGGTGAGGTTCACTGTTTCT, SEQ ID NOs:16 and 17, respectively) was examined by quantitative RT-PCR using Syber Green detection. The relative mRNA expression was normalized by measurement of the amount of Gapdh or β-Actin mRNA or 18S RNA in each sample using Taqman® assays (Applied Biosystems).

Immunoblot analysis: Tissue extracts were prepared using Triton lysis buffer [20 mM Tris (pH 7.4), 1% Triton X-100, 10% glycerol, 137 mM NaCl, 2 mM EDTA, 25 mM β-glycerophosphate, 1 mM sodium orthovanadate, 1 mM phenylmethylsulfonyl fluoride, and 10 μg/mL of aprotinin and leupeptin]. Extracts (20-50 μg of protein) and immunoprecipitates were examined by protein immunoblot analysis by probing with antibodies to AKT, phosphoSer³⁰⁸ AKT, phosphoSer⁴⁷³ AKT (Cell Signaling), phosphoThr³⁰⁸ AKT, insulin receptor (Santa Cruz), ERK1/2 (Santa Cruz), phosphotyrosine (4G10, Upstate), JNK1 (Pharmingen), ERK1/2 (Santa Cruz), JNK1/2 (Pharmingen), cFLIP (Alexis), phospho-Ser³⁰⁷ IRS1 (Millipore), Insulin receptor β sub-unit, cleaved PARP, cleaved caspase 3 (Cell Signaling), α-Tubulin, or β-Actin (Sigma). The IRS1 antibody was prepared by immunization of a rabbit with a peptide that corresponds to the carboxy-terminal 14 amino acids of rat IRS1 ([C]YASINFQKQPEDRQ, SEQ ID NO:18). Immunocomplexes were detected by enhanced chemiluminescence (NEN). Quantitation of immunoblots was performed using the Odyssey™ infrared imaging system (LI-COR Biosciences). The amount of total and phospho-JNK1/2, cJun, ERK1/2, p38 MAPK, and AKT in tissue extracts was measured by multiplexed ELISA using a Luminex 200 instrument (Millipore)

Cytokine analysis: Cytokines in plasma, serum, and culture medium were measured by multiplexed ELISA using a Luminex 200 machine (Millipore) and serum mouse adipokine, adiponectin and cytokine kits (Millipore). Insulin and insulin C-peptide in plasma were measured by ELISA using a Luminex 200 machine. Blood glucose was measured with an Ascensia Breeze 2 glucose meter (Bayer). Alanine transaminase (ALT) and aspartate aminotransferase (AST) activity in serum was measured using the ALT and AST Reagent kit (Pointe Scientific) with a Tecan Sapphire Madhumita Das microplate reader (Tecan Trading AG).

Lipid analysis: Total Cholesterol (Cardiocheck PA, PTS, Inc.), HDL and triglycerides (Sigma) were measured, and the amount of LDL was calculated using a Cardiocheck PA (PTS, Inc.). The concentration of free fatty acids (Roche) and glycerol (Sigma) was measured using kits purchased from the indicated suppliers. Triglyceride was measured using kit purchased from Sigma. FPLC analysis of serum lipoproteins was performed by the University of Cincinnati Mouse Metabolic Phenotyping Center (Lipid, Lipoprotein and Glucose Metabolism Core).

Hepatic triglyceride content was measured using livers from mice starved overnight. Total lipids were extracted from liver samples (50 mg) using an 8:1 mixture of chloroform and methanol (4 hrs). The extracts were mixed with 1N sulfuric acid and centrifuged (10 mins). The amount of triglyceride was measured using a kit purchased from Sigma.

Fat absorption: Fat absorption in mice was determined by the University of Cincinnati Mouse Metabolic Phenotyping Center (Lipid, Lipoprotein and Glucose Metabolism Core) using the sucrose polybehenate method of Jandacek et al., Gastroenterology 127, 139 (2004). The mice were fed the test diet for 5 days. Individually housed mice were transferred to new cages on day 3 and fecal pellets were collected each day. The fecal pellets collected on day 4 and day 5 were examined for the fatty acid content by gas chromatography. Since behenic acid is essentially absent from the fat sources used in the test diet and is entirely excreted when given as sucrose polybehenate, absorption is calculated from the difference between diet and feces in the ratio behenate/total fatty acid.

Hepatic lipogenesis: The mice were fed a standard chow diet and starved for 6 hours. The mice were administered 20 μCi of [3-³H]glucose (PerkinElmer) by intraperitoneal injection and euthanized after one hour. Lipids were extracted from samples of the liver (50 mg) using an 8:1 mixture of chloroform and methanol. The amount of radioactivity incorporated into lipid was measured by liquid scintillation counting.

Protein kinase assays: JNK activity was measured using an in vitro protein kinase assay with the substrates cJun and [γ-32P]ATP as substrates as described by Whitmarsh and Davis, Methods Enzymol 332, 319 (2001). The amount of total and phospho-JNK1/2, cJun, ERK1/2, p38 MAPK, and AKT in tissue extracts was measured by multiplexed ELISA using a Luminex 200 instrument (Millipore).

Glucose tolerance, insulin tolerance, pyruvate challenge, and insulin clearance tests: The mice were fed a standard chow diet or a high fat diet (Iso Pro 3000, Purina and F3282, Bioserve Inc.) for 16 wks. Glucose tolerance tests (GTT), insulin tolerance tests (ITT), and pyruvate challenge tests were performed using methods described by Mora et al., FEBS Lett., 579, 3632 (2005). For GTT, mice fasted overnight were injected intraperitoneally with glucose (1 mg/g) and blood glucose concentration was measured over time. For ITT, mice fed ad libitum were injected intraperitoneally with insulin (0.75 mU/g) and blood glucose concentration was measured over time. In some ITT experiments, blood glucose concentration was measured at 30 minutes and the area under the curve (AUC) was calculated. For pyruvate challenge tests, mice fasted overnight were injected intraperitoneally with pyruvate (1 mg/g) and blood glucose concentration was measured over time.

For insulin clearance assays, mice were administered human insulin (1.5 mU/g; Novolin; Novo Nordisk. Princeton N.J.) by intraperitoneal injection. The amount of human insulin in the blood at different times postinjection was measured by ELISA (Luminex 200 machine, Millipore).

Hyperinsulinemic-euglycemic clamp studies: These assays were performed at the Penn State Diabetes & Obesity Mouse Phenotyping Center. Briefly, F^{WT} and F^{KO} mice were fed a HFD diet (55% fat by calories; Harlan Teklad) or chow diet for 3 weeks, and whole body fat and lean mass were non-invasively measured using ¹H-MRS (Echo Medical Systems). Following an overnight fast, a 2-hr hyperinsulinemiceuglycemic clamp was conducted in awake mice with a primed (150 mU/kg body weight priming) and continuous infusion of human insulin (2.5 mU/kg/min; Humulin; Eli Lilly), and 20% glucose was infused at variable rates to maintain euglycemia (Kim et al., Diabetes, 53, 1060 (2004)). Whole body glucose turnover was assessed with a continuous infusion of [3-³H]glucose and 2-deoxy-D[1-¹⁴C]glucose (PerkinElmer) was administered as a bolus (10 μCi) at 75 min after the start of clamps to measure insulin-stimulated glucose uptake in individual organs. At the end of the clamps, mice were anesthetized, and tissues were taken for biochemical analysis as described by Kim et al., 2004, supra.

Analysis of tissue sections: Sections (7 μm) prepared from tissue frozen in O.C.T. compound (Tissue-Tek) were stained with Oil-red-O (Sigma). Tissue fixed in 4% paraformaldehyde (24 h) was processed and embedded in paraffin. Sections (5 μm) were prepared and mounted on coverslips for staining with haematoxylin & eosin or by TUNEL assay using an in situ cell death kit (Roche). De-parafinized sections (4 μm) were also stained, following microwave antigen retrieval and incubation (1h, 25° C.) in Tris-buffered saline supplemented with 0.4% Triton and 10% goat serum, with an ALEXA FLUOR 647-conjugated antibody (clone CI:A3-1; Serotech) to the macrophage antigen F4/80 (16 h, 4° C.). The sections were washed, the coverslips were mounted on slides in medium with DAPI (Vector Labs), and examined by confocal fluorescence microscopy (Leica). Hepatic damage detected in stained liver sections was quantitated using ImagePro Plus software (Media Cybernetics).

In some embodiments, histology was performed using tissue fixed in 10% formalin for 24 h, dehydrated and embedded in paraffin. Sections (7 μm) were cut and stained using hematoxylin and eosin (American Master Tech Scientific). Immunohistochemistry was performed by staining tissue sections with an antibody to F4/80 (Abcam), a biotinylated secondary antibody (Biogenex), streptavidinconjugated horseradish peroxidase (Biogenex), and the substrate 3,3′-diaminobenzidene (Vector Laboratories) followed by brief counter-staining with Mayer's hematoxylin (Sigma).

Metabolic cages: Male mice were housed under controlled temperature and lighting with free access to food and water. The food/water intake, energy expenditure, respiratory exchange ratio, and physical activity were performed (3 days) using metabolic cages (TSE Systems, Bad Homburg, Germany).

Flow cytometry: Peripheral blood leukocytes and splenocytes (10⁶ cells) were incubated with anti-CD32/CD16 antibodies to block Fc receptors and then stained with PE-conjugated anti-CD4, APC-conjugated anti-CD8 plus FITC-conjugated anti-B220 antibody (Pharmingen) or with PE-conjugated anti-CD45.1 plus FITC-conjugated anti-CD45.2 (Pharmingen) in phosphate-buffered saline plus 2% serum. Flow cytometry was performed using a FACScan cytofluorometer (Becton Dickinson) and data were examined using FlowJo software.

Statistical analysis: Differences between groups were examined for statistical significance using the Student's test, analysis of variance (ANOVA) with the Bonferroni post-test, ANOVA with the Fisher's test, or the log-rank test.

### Example 2

### Role of JNK1 During Development of Diet-Induced Insulin Resistance

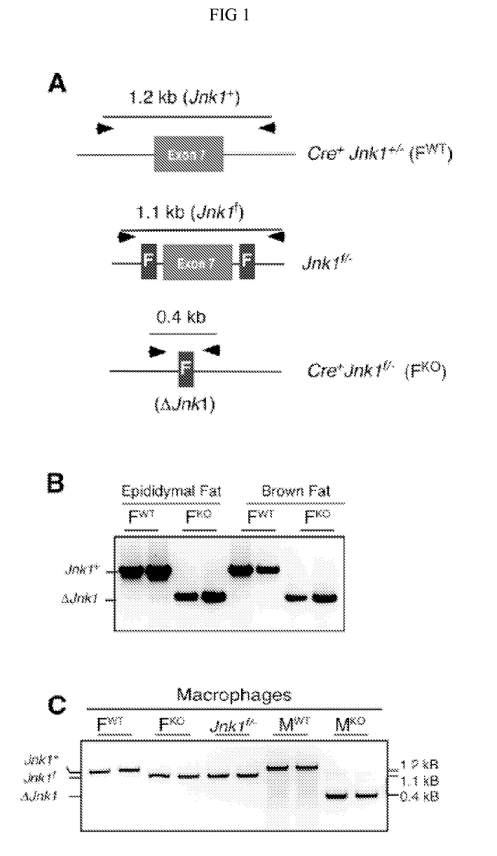

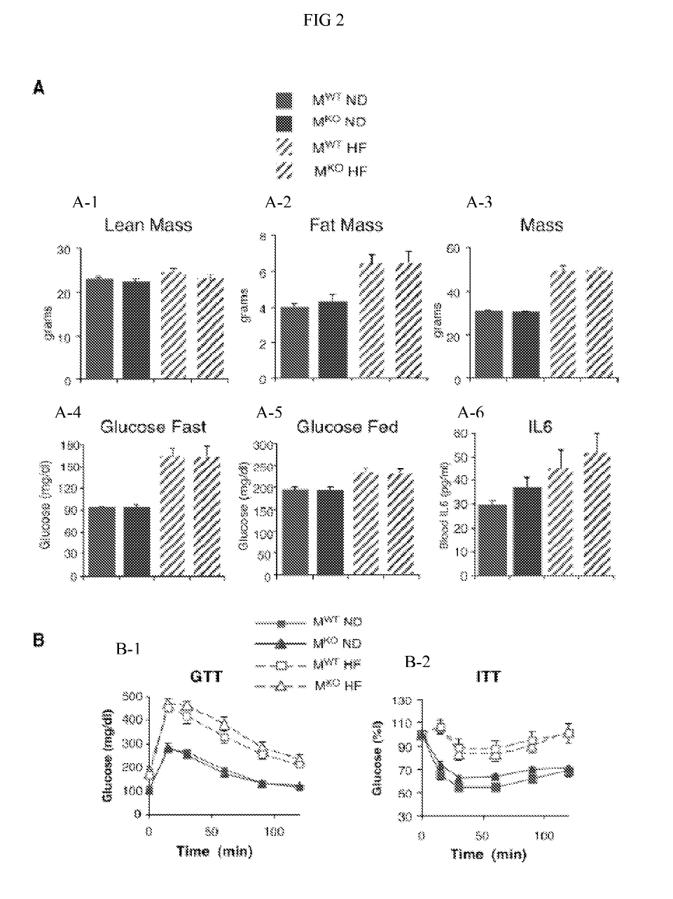

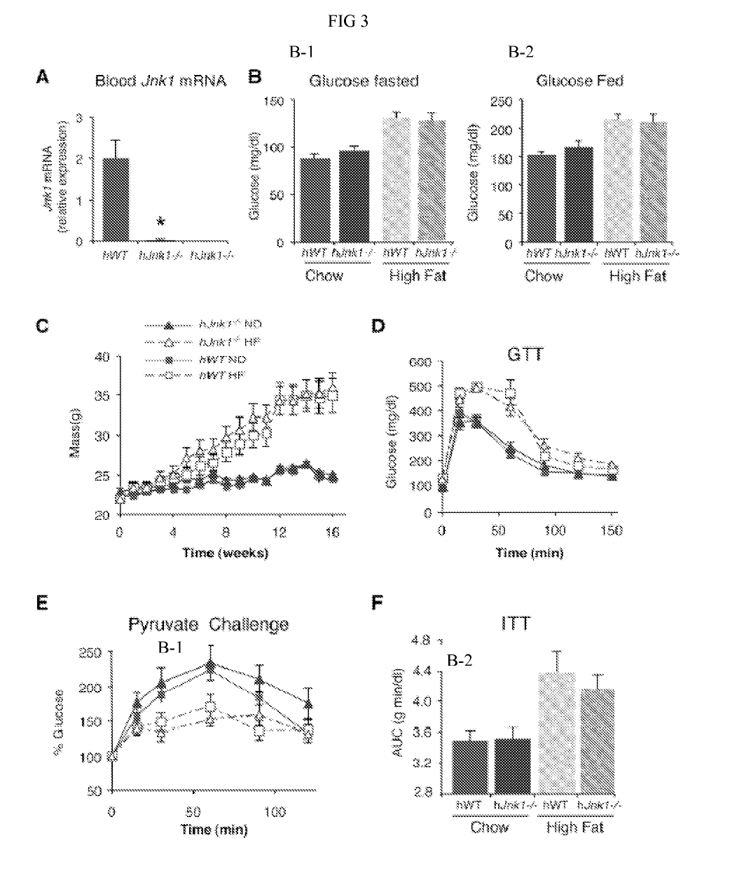

To test the role of JNK1 in myeloid cells during the development of diet-induced insulin resistance, the phenotype of mice with JNK1-deficiency in myeloid cells (FIGS. 1 and 2) and hematopoietic cells (FIG. 3) was examined. No significant difference in the response of these JNK1-deficient HFD-fed mice, compared with control HFD-fed mice, was detected in glucose and insulin tolerance tests (FIGS. 2 and 3). These data indicate that, although JNK1 in hematopoietic cells may contribute to HFD-induced insulin resistance, other cell types must also participate in the development of insulin resistance. Adiposity is known to influence insulin responsiveness (Carey et al., Diabetes, 45, 633 (1996)) through a mechanism that involves adipose-derived fatty acids and hormones/cytokines (collectively termed “adipokines”) that can modulate insulin sensitivity (Waki and Tontonoz, Annu. Rev. Pathol., 2, 31 (2007)).

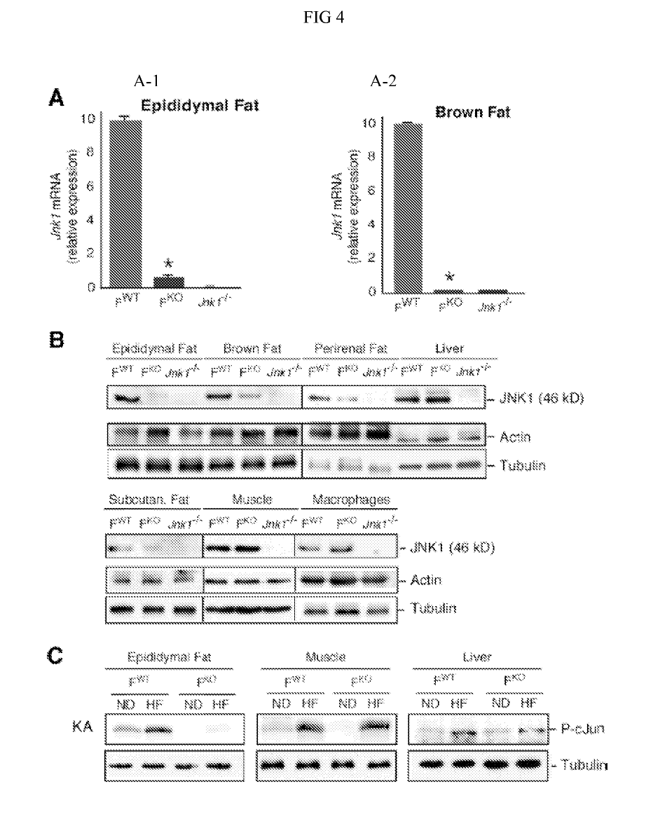

The role of JNK1 in adipocytes on the regulation of insulin sensitivity was tested as follows. Mice lacking JNK1 in adipose tissue (F^{KO}) were generated using animals with conditional (floxed) Jnk1 and adipose tissue-specific expression of Cre recombinase (Fabp4-Cre+Jnk1^{f/-}) as set forth in Example 1. Littermates without conditional Jnk1 (Fabp4-Cre+Jnk1^{+/-}) were used as control mice (F^{WT}). The Jnk1⁺, Jnk1^{f}, and deleted Jnk1 (ΔJnk1) alleles were detected by PCR amplification of genomic DNA (FIG. 1A). Efficient deletion of Jnk1f was detected in the adipose tissue of FKO mice (FIG. 1B). In contrast, Jnk1f was not deleted in other tissues of FKO mice, including macrophages (FIG. 1C). Quantitative PCR analysis demonstrated that Jnk1 mRNA was markedly reduced in epididymal fat and brown fat of FKO animals (FIG. 4A). Immunoblot analysis confirmed the reduction of JNK1 protein in fat depots from F^{KO} mice, while JNK1 was preserved in liver, muscle, and macrophages (FIG. 4B). JNK1 is activated in mice following exposure to metabolic stress (Weston and Davis, Curr. Opin. Cell Biol., 19, 142 (2007)). Indeed, it was found that JNK1 was activated in the adipose tissue, striated muscle, and liver of HFD-fed F^{WT} mice (FIG. 4C). In contrast, HFD-fed F^{KO} mice exhibited JNK activation in muscle and liver, but not adipose tissue (FIG. 4C). Together, these data indicate that F^{KO} mice are useful for studies of the role of JNK1 in adipose tissue.

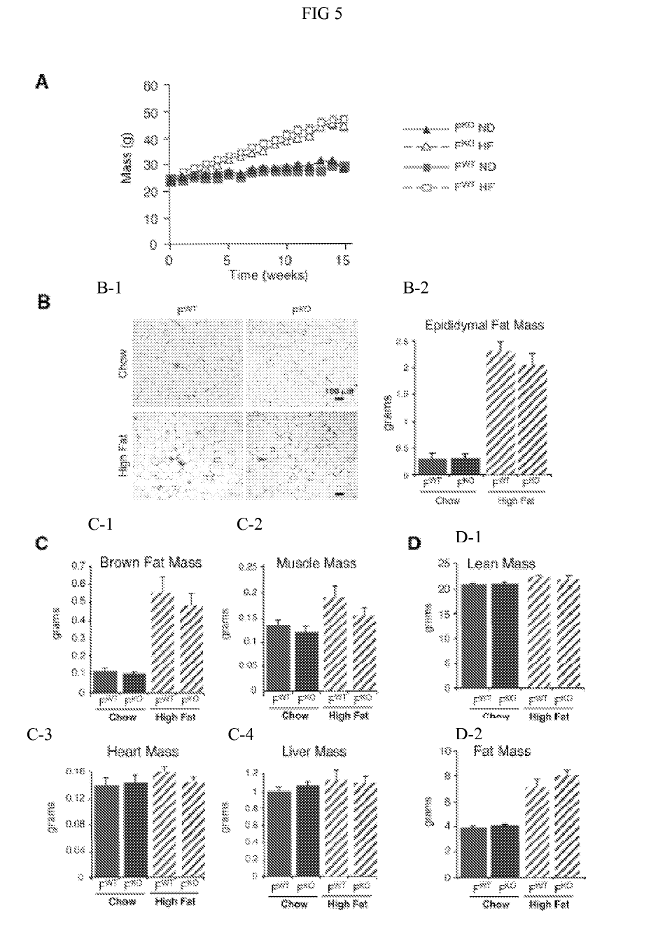

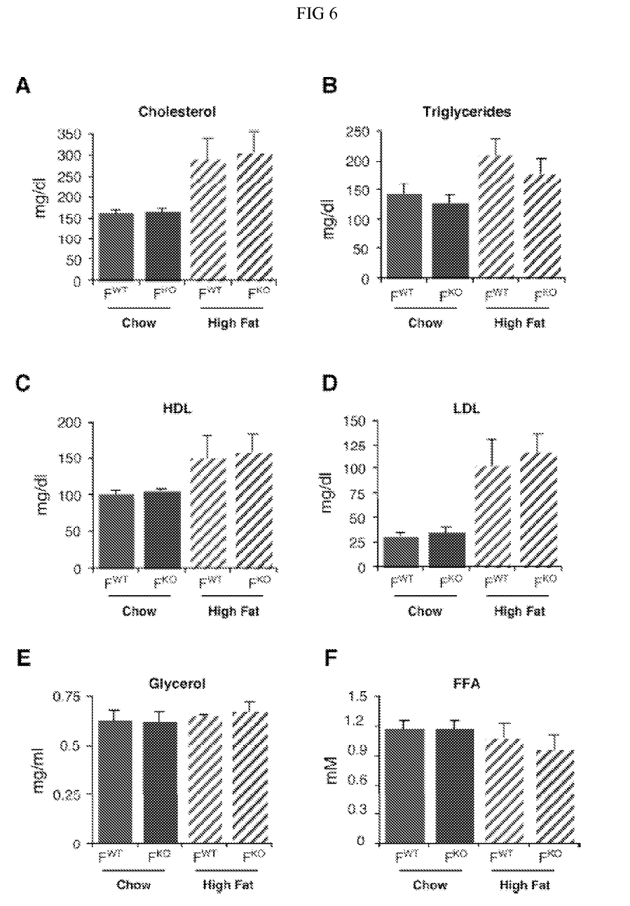

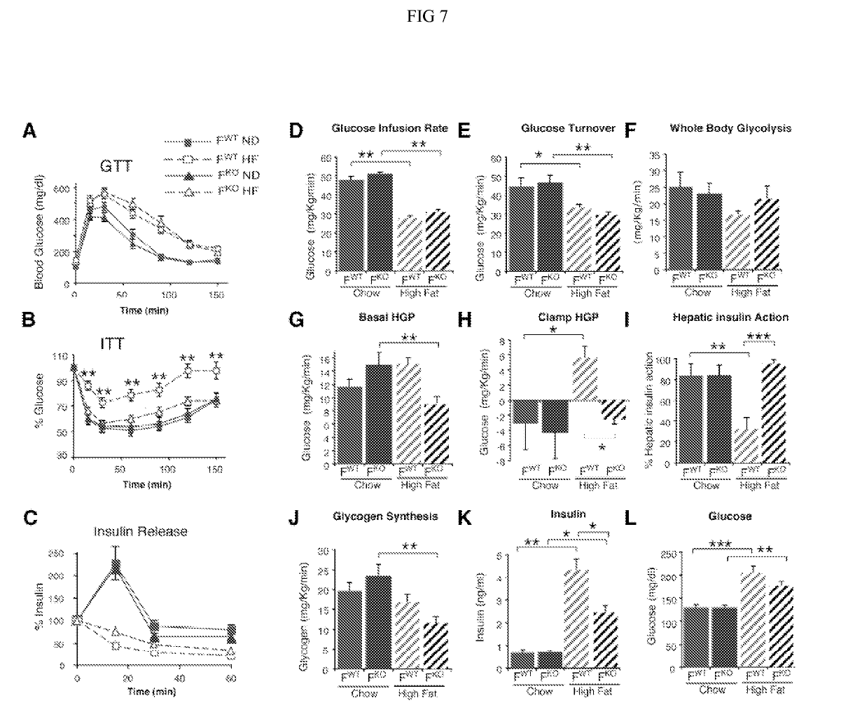

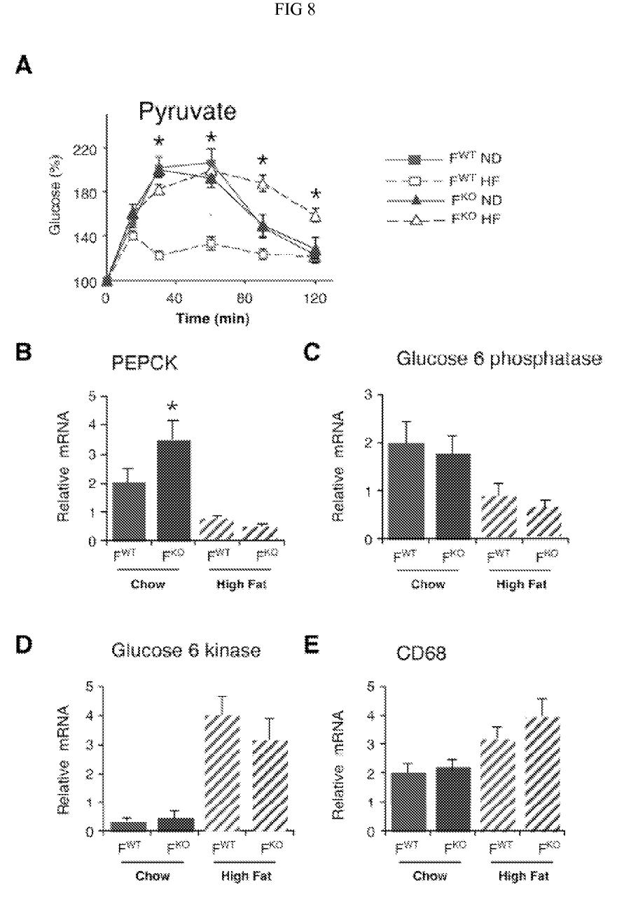

Comparison of HFD-fed F^{WT} and F^{KO} mice demonstrated that these animals gained similar body mass (FIG. 5A-5D) and blood lipids (FIG. 6A-6F), became glucose intolerant (FIG. 7A) with reduced glucose-induced insulin secretion (FIG. 7C), and developed mild fasting hyperglycemia (FIG. 7L). In contrast, when compared to HFD-fed F^{WT} mice, the HFD-fed F^{KO} mice showed improved insulin sensitivity during an insulin tolerance test (FIG. 7B) and reduced hyperinsulinemia (FIG. 7K). A 2-hr hyperinsulinemic-euglycemic clamp study was performed to assess organ-specific glucose metabolism in awake F^{WT} and F^{KO} mice. After 3 weeks of HFD, both groups of mice developed whole body insulin resistance, as indicated by significant reductions in glucose infusion rate and whole body glucose turnover during the clamp (FIG. 7D, 7E). HFD-fed F^{WT} mice developed insulin resistance in liver, as indicated by increased hepatic glucose production (HGP) during the clamp, but HFD-fed F^{KO} mice remained insulin sensitive in liver (FIG. 7H, 7I). Basal HGP was not affected by feeding a HFD or by JNK1 deletion in adipose tissue (FIG. 7G). Studies of hepatic gluconeogenesis demonstrated that increased blood glucose caused by pyruvate administration was suppressed in HFD-fed F^{WT} mice, but not HFD-fed F^{KO} mice (FIG. 8A). No differences in whole body glycolysis or glycogen synthesis were detected between HFD-fed F^{WT} and F^{KO} mice (FIG. 8F,8J). Together, these data demonstrate that adipose-specific disruption of the Jnk1 gene prevents diet-induced hepatic insulin resistance.

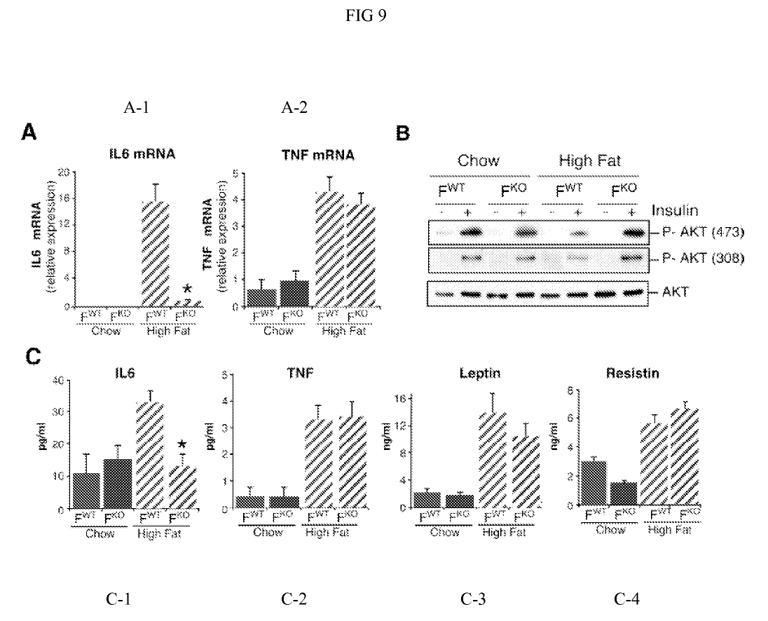

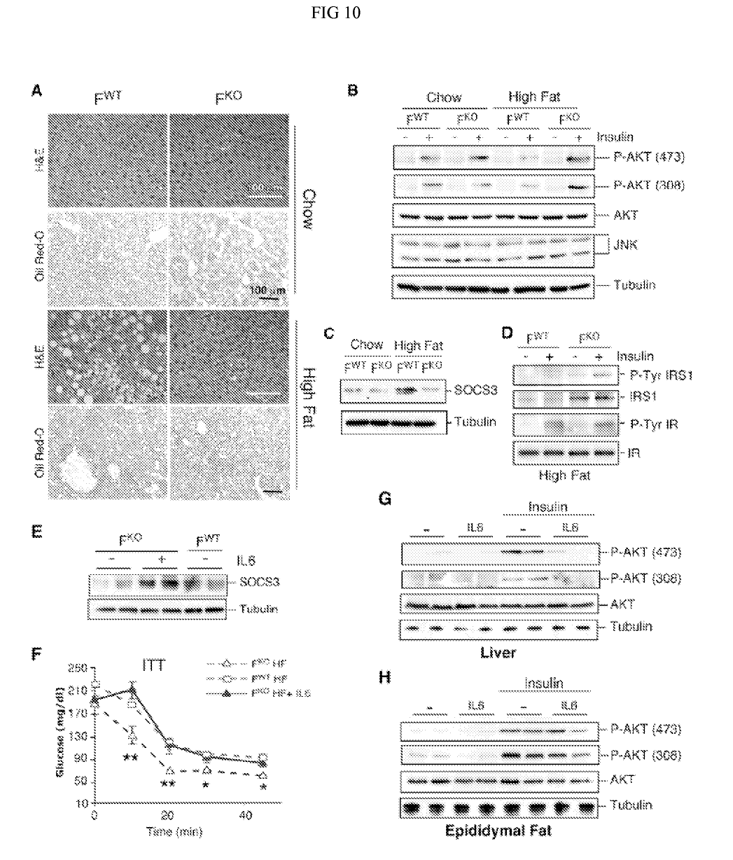

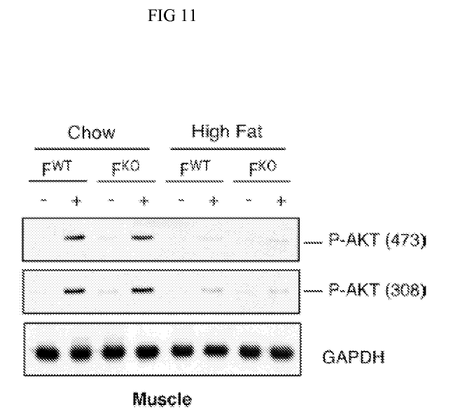

To confirm the effect of adipose JNK1-deficiency on insulin sensitivity, insulin-stimulated Ser/Thr phosphorylation and activation of AKT was tested. HFD-fed F^{WT} mice exhibited reduced insulin-stimulated AKT activation in adipose tissue (FIG. 9B), liver (FIG. 10B), and muscle (FIG. 11). In contrast, F^{KO} mice exhibited HFD-induced inhibition of insulin-stimulated AKT activation in muscle (FIG. 11), but not in adipose tissue or liver (FIGS. 9B & 10B). This effect of adipose-specific JNK1-deficiency on hepatic AKT activation (FIG. 10B) is consistent with the observation that HFD-fed F^{KO} mice showed improved hepatic insulin sensitivity compared with HFD-fed F^{WT} mice (FIG. 7I). Interestingly, HFD-fed F^{KO} mice also exhibited reduced hepatic steatosis compared to HFD-fed F^{WT} mice (FIG. 10A). These data confirm that JNK1 in adipose tissue is, at least in part, required for HFD-induced insulin resistance in both adipose tissue and liver.

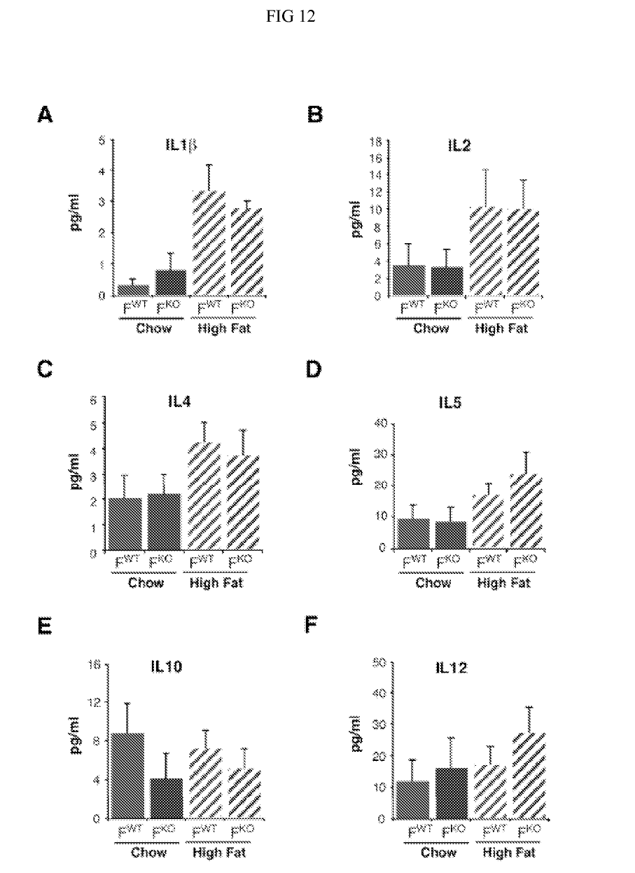

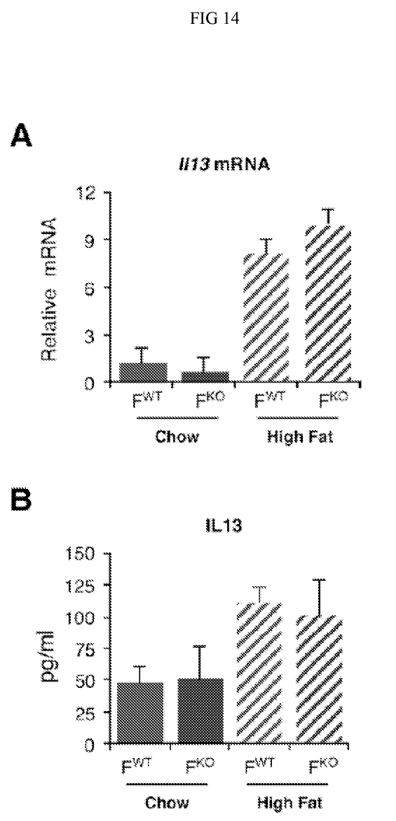

The total fat mass, weight of the epididymal fat pads, and the size of adipocytes were not significantly different between HFD-fed F^{WT} and F^{KO} mice (FIGS. 5A-5D). The HFD increased Tnfα and Il6 mRNA expression in adipose tissue of F^{WT} mice, but only increased Tnfα mRNA expression was detected in F^{KO} mice (FIG. 9A). Moreover, the HFD caused a similar increase in the serum concentration of TNFα in F^{WT} and F^{KO} mice, but increased serum IL6 was only detected in F^{WT} mice (FIG. 9C). Thus, JNK1-deficiency in adipocytes prevented the HFD-induced increase in the expression of the inflammatory cytokine IL6. This effect on IL6 expression was selective, because no significant differences in circulating leptin or resistin concentrations were detected between F^{WT} and F^{KO} mice (FIG. 9C). Furthermore, no significant differences in the serum concentration of other interleukins and adipokines were detected between F^{WT} and F^{KO} mice (FIGS. 12-14).

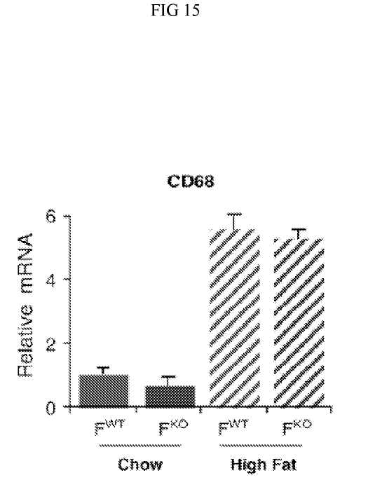

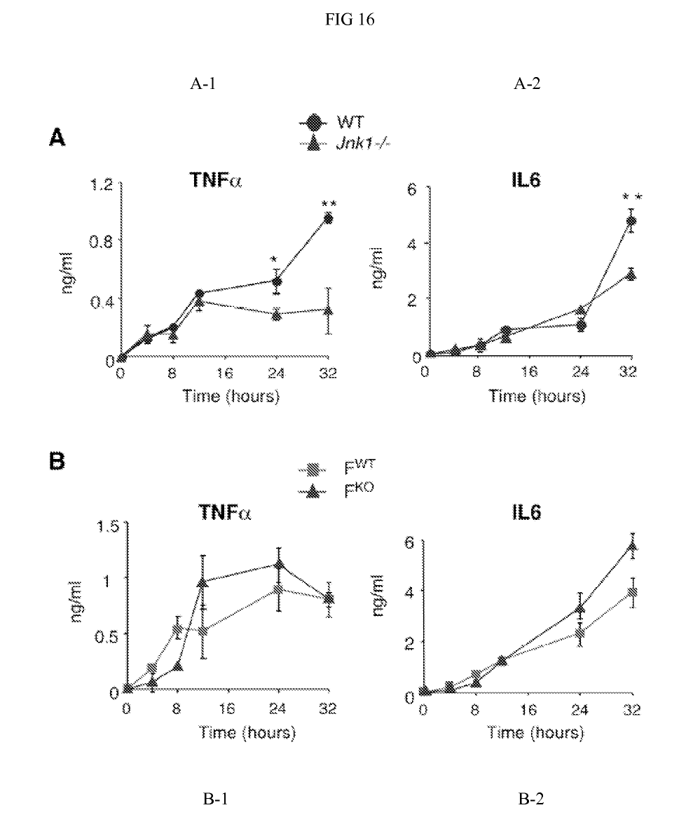

The inflammatory cytokines TNFα and IL6 can cause insulin resistance (Spiegelman and Flier, Cell 87, 377 (1996); Bastard et al., Eur. Cytokine Netw., 17, 4 (2006)), and JNK can regulate the expression of both cytokines (Weston and Davis, 2007, supra). However, JNK1-deficiency in adipose tissue selectively prevented HFD-induced IL6 expression (FIG. 9A,9C). This finding suggests that adipocytes play a primary role in obesity-induced IL6 expression (Mohamed-Ali et al., J. Clin. Endocrinol. Metab. 82, 4196 (1997)). In contrast, macrophages may represent the major source of TNFα expression (Weisberg et al., J. Clin. Invest. 112, 1796 (2003)). No differences in macrophage infiltration of the liver and adipose tissue were detected between HFD-fed F^{WT} and F^{KO} mice (FIGS. 8E, 13D, 15). Moreover, no defects in IL6 or TNFα expression by macrophages isolated from FKO mice were detected (FIG. 16).

IL6 can induce hepatic insulin resistance (Klover et al., Diabetes, 52, 2784 (2003); Kim et al., Diabetes, 53, 1060 (2004)), and loss of IL6 selectively improves hepatic insulin action in obese mice (Klover et al., Endocrinology, 146, 3417 (2005)). IL6-induced hepatic insulin resistance is mediated, in part, by increased expression of SOCS3 (Senn et al., J. Biol. Chem., 278, 13740 (2003); Torisu et al., Genes Cells, 12, 143 (2007)), a protein that binds and inhibits the insulin receptor (Emanuelli et al., J. Biol. Chem., 276, 47944 (2001); Emanuelli et al., J. Biol. Chem., 275, 15985 (2000)) and also targets insulin receptor substrate (IRS) proteins for proteosomal degradation (Rui et al., J. Biol. Chem., 277, 42394 (2002)).

Expression of SOCS3 was increased and IRS1 was decreased in the liver of HFD-fed F^{WT} mice, but not HFD-fed F^{KO} mice (FIG. 10C,D). Dysregulated expression of SOCS3 and IRS1 in the liver of HFD-fed F^{KO} mice is consistent with the observation that HFD-fed F^{KO} mice exhibit a low circulating concentration of IL6 (FIG. 9C) and improved hepatic insulin sensitivity (FIG. 7I) compared to HFD-fed F^{WT} mice.

To test whether the defect in adipose tissue expression of IL6 contributes to the improved hepatic insulin sensitivity of HFD-fed F^{KO} mice, it was examined whether administration of IL6 would restore HFD-induced insulin resistance phenotypes in F^{KO} mice. Acute IL6 treatment increased hepatic SOCS3 expression in HFD-fed F^{KO} mice to the same amount that was detected in HFD fed F^{WT} mice (FIG. 10E). Insulin tolerance tests demonstrated that IL6-treated HFD-fed F^{KO} mice became equally insulin resistant as HFD-fed F^{WT} mice (FIG. 10F). Moreover, IL6 treatment reduced insulin-stimulated AKT activation in the liver of HFD-fed F^{KO} mice (FIG. 10G). In contrast, only a moderate effect of IL6 on AKT activation in the adipose tissue of HFD-fed F^{KO} mice was detected (FIG. 10H). These data demonstrate that the effect of JNK1-deficiency in adipose tissue on hepatic insulin sensitivity is, at least in part, mediated by a requirement of JNK1 for HFD-induced expression of IL6.

Adipose tissue plays a critical role in glucose homeostasis by releasing adipokines that regulate insulin sensitivity in other organs (Spiegelman and Flier, Cell, 87, 377 (1996); Mauvais-Jarvis et al., Clin Endocrinol. (Oxf), 57, 1 (2002)). One of these, IL6, is elevated in obese, diabetic subjects, and regulates glucose metabolism in multiple cell types (Kishimoto, Blood, 74, 1 (1989); Senn et al., Diabetes 51, 3391 (2002); Pedersen et al., Curr. Opin. Hematol., 8, 137 (2001)). However, the role of IL6 in whole body insulin resistance has been debated, because IL6 alters insulin signaling differently in individual tissues (Pedersen et al., Curr. Opin. Hematol., 8, 137 (2001); Mooney, J. Appl. Physiol., 102, 816 (2007); Pedersen and Febbraio, J. Appl. Physiol., 102, 814 (2007)). Furthermore, IL6 regulates the hypothalamic-pituitaryadrenal axis (Wallenius et al., Nat. Med., 8, 75 (2002)) and the IL6/Stat3 pathway is required for the action of insulin signaling in the brain on hepatic gluconeogenesis (Inoue et al., Cell Metab., 3, 267 (2006).). Thus, IL6 has both central and peripheral roles on metabolism and its effects on systemic insulin resistance are complex. Nevertheless, neutralization of IL6 selectively improves obesity-induced hepatic insulin resistance and treatment with IL6 increases hepatic insulin resistance. Moreover, ablation of the IL6 target gene Socs3 in the liver of young mice causes improved hepatic insulin sensitivity.

Together, these data and experiments described herein demonstrate that adipose tissue-derived IL6 is an important mediator of hepatic insulin resistance and that JNK1 is a component of a metabolic stress signaling pathway that regulates IL6 expression in adipose tissue.

### Example 3

### Examination of Hepatitis in Mice Deficient of JNK1 or JNK2

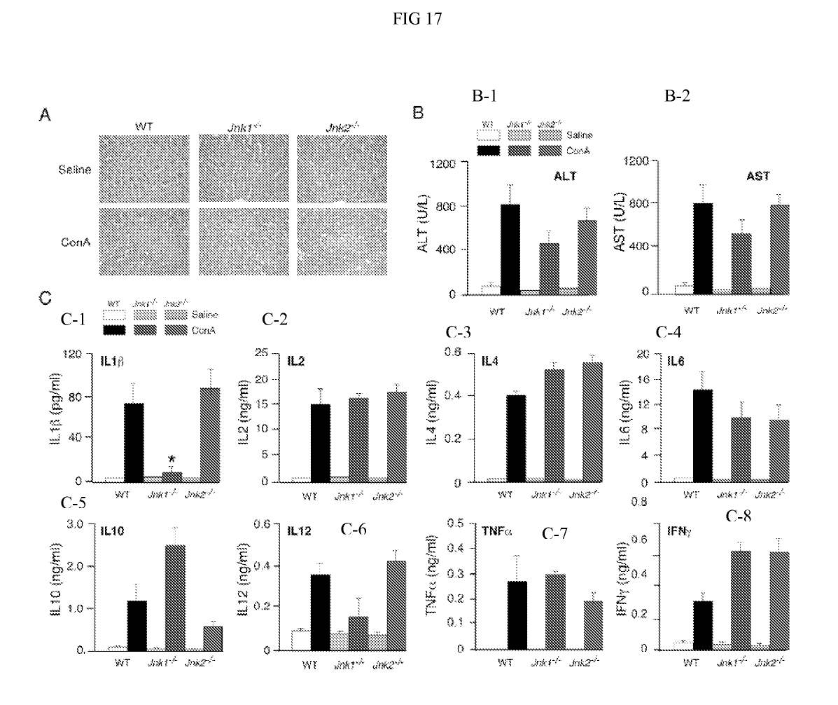

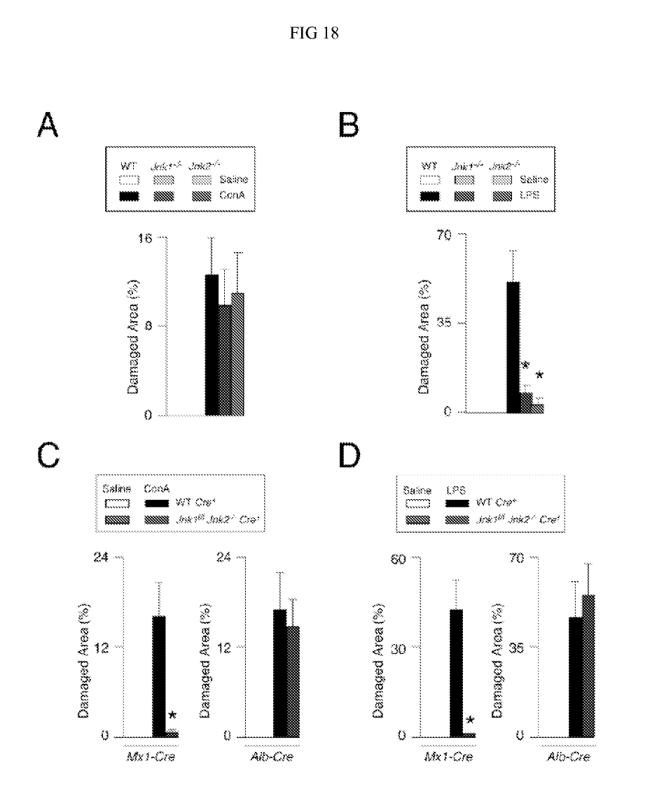

The effect of ConA-induced hepatitis on wild-type, Jnk1^{-/-}, and Jnk2^{-/-} mice was assessed as set forth in Example 1. ConA caused similar hepatic damage in wild-type and JNK knockout mice (FIGS. 17A & 18A). Serum transaminase activity, a hallmark of hepatic injury, was modestly reduced in both Jnk1^{-/-} mice and Jnk2^{-/-} mice compared to wild-type mice, but the differences were not statistically significant (FIG. 18B). The effects of ConA on hepatic injury are thought to be mediated by induced cytokine expression. Similar amounts of serum cytokines were detected in the wild-type and knockout mice, although a selective loss of IL-1 expression was found in Jnk1^{-/-} mice (FIG. 18C). Together, these data suggest that JNK1 and JNK2 do not play a major role in ConA-stimulated hepatitis.

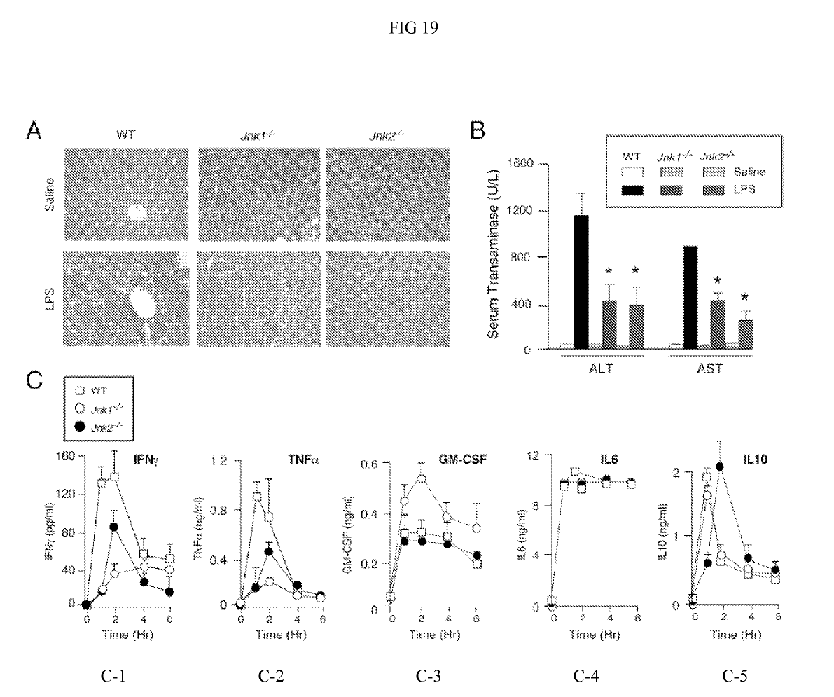

Nevertheless, it was possible that JNK may play a role because studies using a different model of murine hepatitis (treatment with LPS) did show that both Jnk1^{-/-} mice and Jnk2^{-/-} mice exhibited a partial reduction in hepatic damage that was confirmed by histological analysis of liver sections (FIGS. 18B & 19A), measurement of serum transaminase activity (FIG. 19B), and decreased expression of serum cytokines, including TNFα and IFNγ (FIG. 19C).

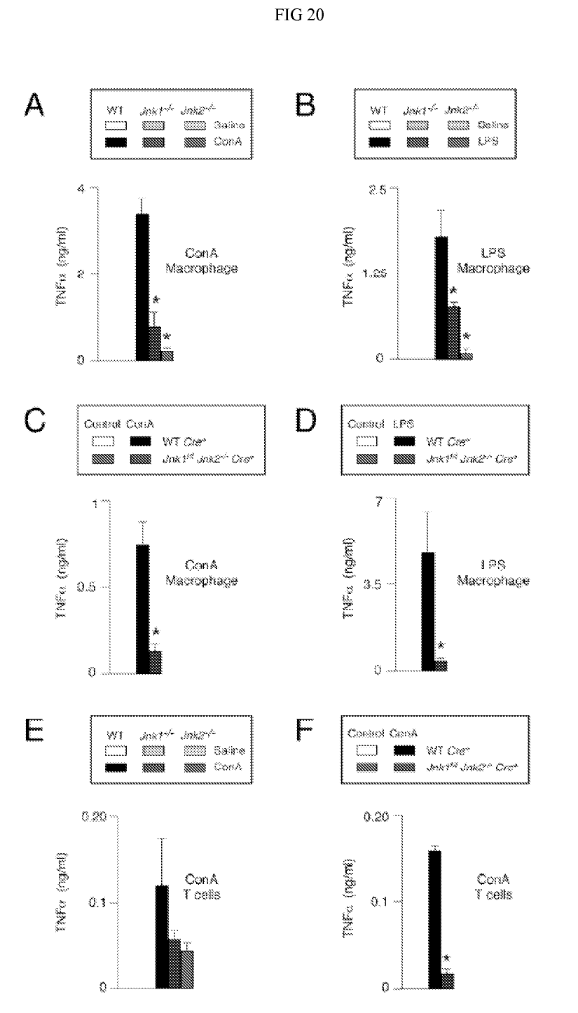

The analysis of hepatitis in Jnk1^{-/-} mice and Jnk2^{-/-} mice indicated that JNK1 and JNK2 may contribute to the disease process (FIGS. 17-19). However, the roles of JNK1 and JNK2 are unclear because it was found that Jnk1^{-/-} mice and Jnk2^{-/-} mice did not demonstrate defects in ConA-induced hepatitis (FIG. 17). These observations suggested the possibility that JNK1 and JNK2 may play redundant roles during the development of hepatitis (FIG. 20).

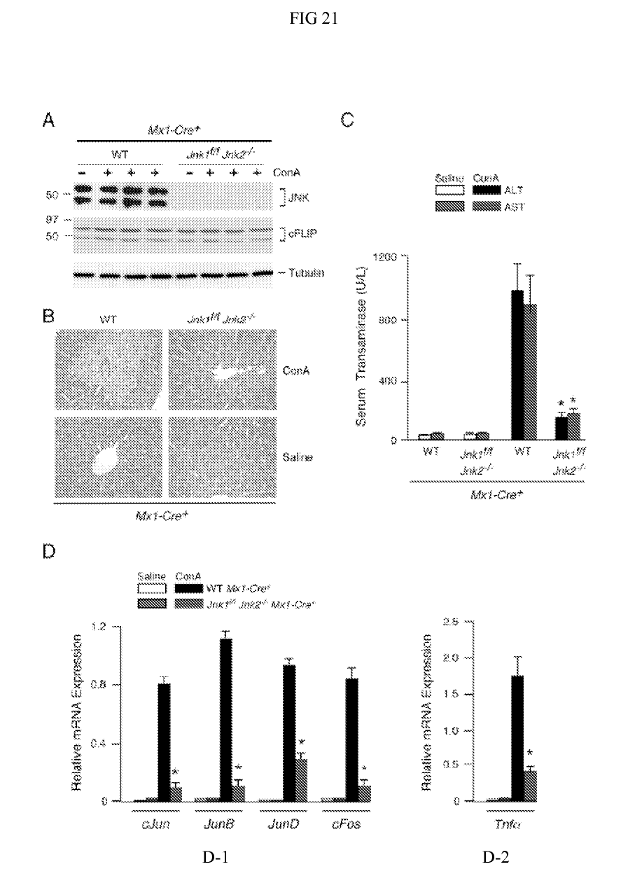

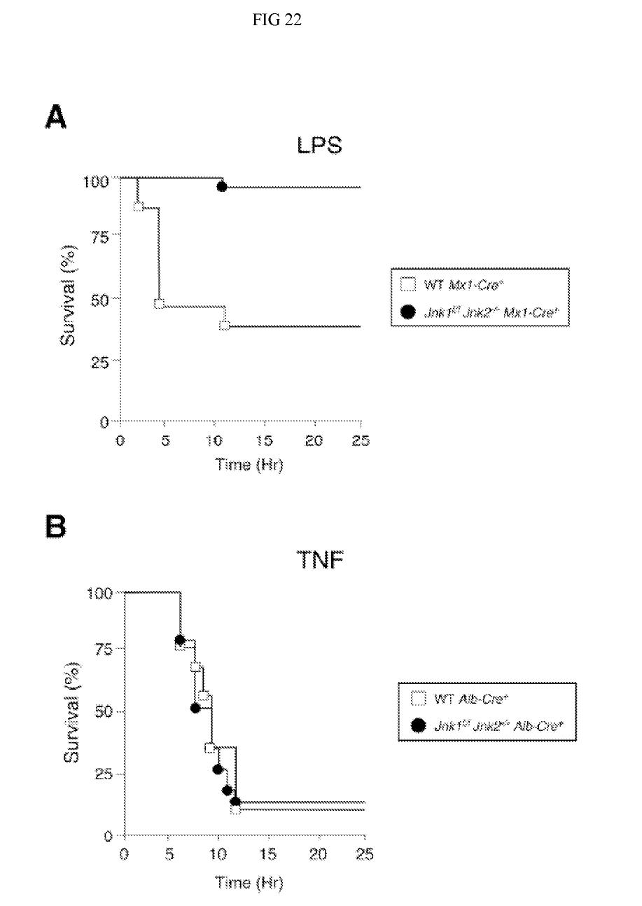

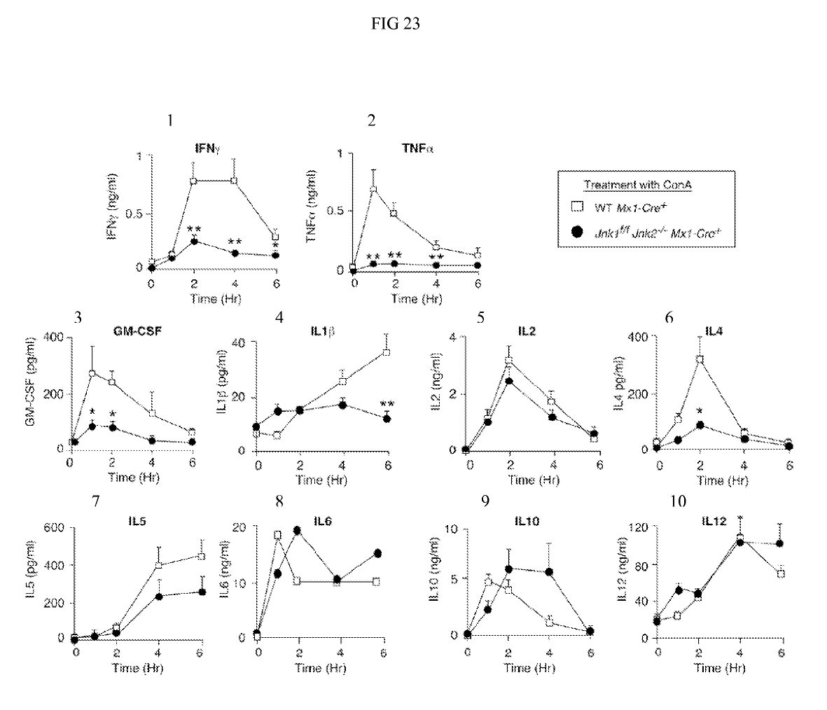

To test this hypothesis, the effect of ConA in mice with compound disruption of both the Jnk1 and Jnk2 genes was examined. Since Jnk1^{-/-}Jnk2^{-/-} mice die during early embryonic development (Kuan et al., Science, 269, 1427 (1999)), a conditional gene ablation strategy was employed. Efficient deletion of floxed alleles in both the liver and hematopoietic cells can be achieved in Mx1-Cre transgenic mice when these mice are treated with polyIC (Kuhn et al., 1995, supra). Mx1-Cre⁺ (Control) mice and Mx1-Cre⁺ Jnk1^{f/f}Jnk2^{-/-} mice were treated with polyIC (FIG. 21). Deletion of floxed Jnk1 (Jnk1^{Δ/Δ}) was confirmed by PCR analysis of genomic DNA. JNK protein was not detected in the liver of the compound mutant mice by immunoblot analysis (FIG. 21A). Treatment of these mice with ConA demonstrated that the JNK1/2-deficient mice exhibited strong protection against hepatitis when examined by histological analysis of liver sections (FIGS. 21B & 18C) or measurement of serum transaminase activity (FIG. 21C). The JNK1/2-deficient mice also exhibited reduced mortality compared with control mice in response to the ConA challenge (FIG. 22A). Studies of hepatic gene expression demonstrated that the Jnk1^{Δ/Δ}Jnk2^{-/-} liver exhibited defects in the ConA-induced expression of several AP1-related genes (FIG. 21D). This observation suggested that the Jnk1^{Δ/Δ}Jnk2^{-/-} liver may exhibit profound defects in AP1-dependent gene expression. Indeed, expression of the AP1 target gene Tnfα was profoundly reduced in the liver of ConA-treated JNK1/2-deficient mice (FIG. 21D). This loss of TNFα expression was confirmed by measurement of the concentration of serum cytokines ConA-induced the expression of many cytokines in the serum of control mice, but JNK1/2-deficient mice exhibited major defects in serum cytokine expression, including TNFα (FIG. 23).

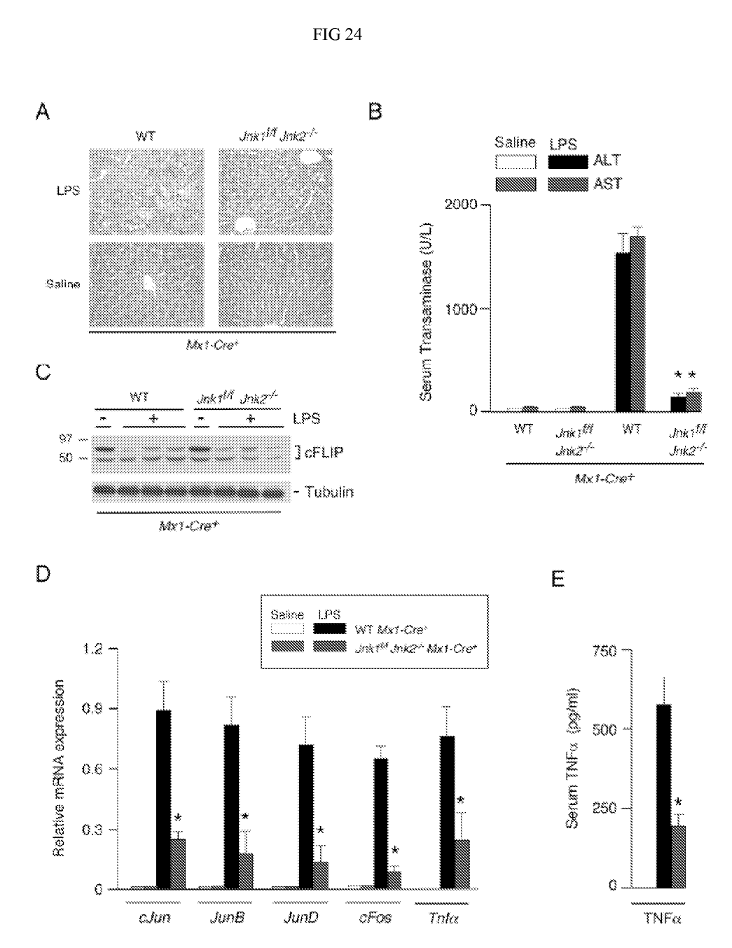

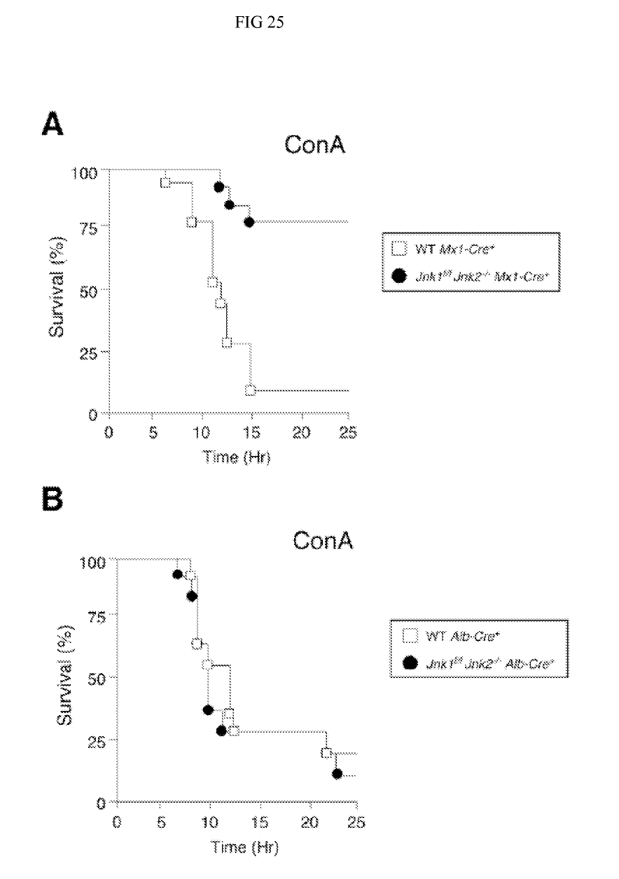

The protection of Mx1-Cre⁺Jnk1^{f/f}Jnk2^{-/-} mice against hepatitis (FIGS. 21 & 18) was confirmed by studies using the LPS model of hepatitis. JNK1/2-deficiency prevented LPS-induced hepatic damage detected by histological analysis of liver sections (FIGS. 18D & 24A), and measurement of serum transaminase activity (FIG. 24B). Decreased expression of AP1-related genes and Tnfα mRNA in the liver (FIG. 24D) and decreased amounts of serum TNFα also were detected in the JNK1/2-deficient mice (FIG. 24E). The JNK1/2-deficient mice also exhibited reduced mortality compared with control mice in the response to LPS challenge (FIG. 25A). Together, these data indicate that compound deficiency of both JNK1 and JNK2 caused strong protection against two different models of hepatitis that are induced by treatment of mice with ConA or LPS, respectively.

### Example 4

### JNK-Deficiency in Hepatocytes does not Protect Mice Against Hepatitis

The observation that compound mutation of Jnk1 and Jnk2 causes protection against both ConA- and LPS-induced hepatitis (FIGS. 18, 21, & 24) suggested that JNK1 and JNK2 may play redundant roles during progression of the disease process. It has been proposed that these models of hepatitis are mediated by TNFα-induced and JNK-mediated phosphorylation and activation of the E3 ligase Itch and the subsequent ubiquitin-mediated degradation of the caspase 8 antagonist cFLIP in hepatocytes (Chang et al., Cell, 124, 601 (2006)). To test this hypothesis, cFLIP was examined in control and JNK1/2-deficient liver of mice treated with ConA; no changes in cFLIP expression were detected (FIG. 21A). In contrast, treatment with LPS did cause cFLIP degradation, but this degradation of cFLIP was JNK-independent (FIG. 24C). The observation that LPS, but not ConA, caused cFLIP degradation may reflect the more potent effects of LPS on hepatitis compared with ConA. Together, these observations indicated that JNK-mediated cFlip degradation is not required for murine models of hepatitis caused by ConA or LPS.

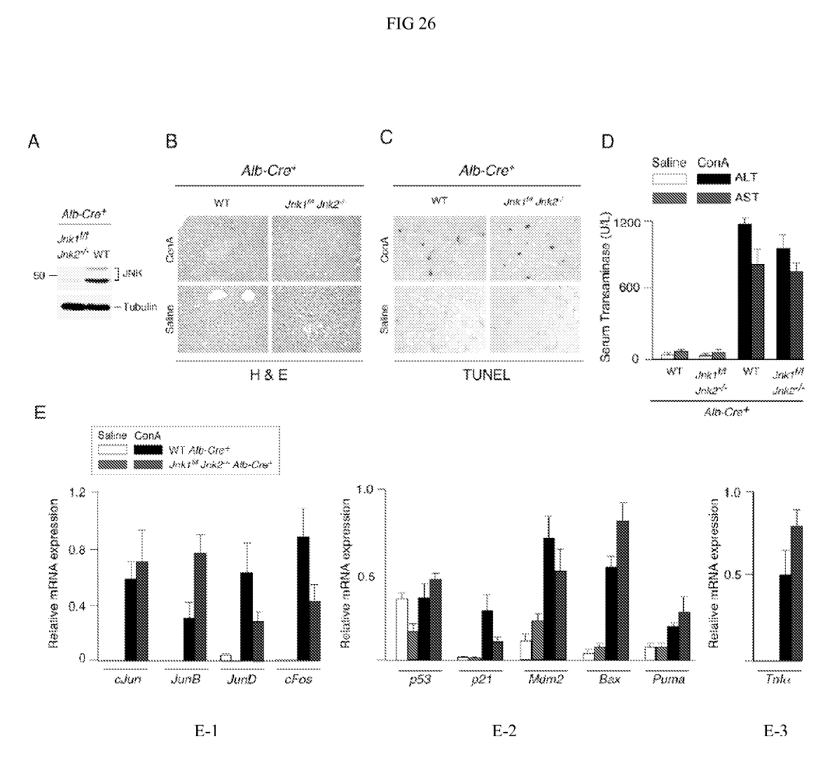

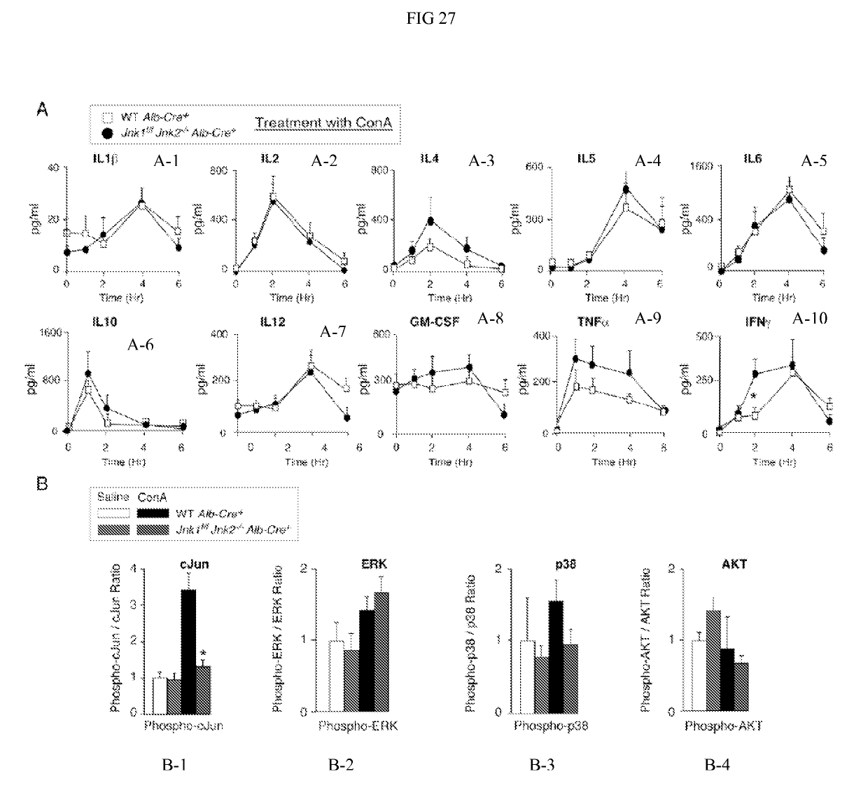

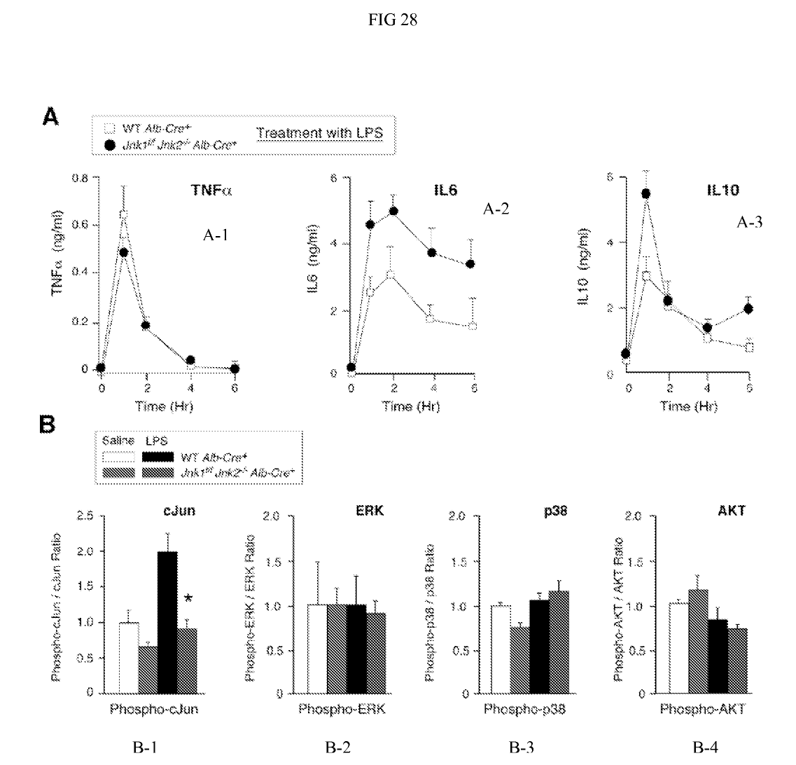

To test the role of JNK in hepatocytes, the effect of hepatocyte-specific JNK-deficiency was examined using Alb-Cre transgenic mice. Immunoblot analysis of liver extracts prepared from control (Alb-Cre⁺) mice and JNK-deficient (Jnk1^{f/f}Jnk2^{-/-}Alb-Cre⁺) mice demonstrated that very low amounts of JNK were detected in JNK-deficient liver (FIG. 26A). The presence of low levels of JNK in the Alb-Cre deleted mice is consistent with the finding that the Alb-Cre transgene is selectively expressed in hepatocytes, but not in other liver cell-types, including stellate cells, endothelial cells, NK cells, NKT cells, and Kupffer cells (Postic et al., 1999, supra). Control studies demonstrated that hepatocyte-specific JNK1/2-deficiency caused markedly reduced hepatic JNK activity (monitored be measurement of phospho-Ser⁷³ cJun) in mice treated with ConA or LPS (FIGS. 27B & 28B).

Treatment with ConA caused similar hepatic damage in control and hepatocyte-specific JNK1/2-deficient mice (FIGS. 26B & 18C). No differences in TUNEL staining of liver sections of the control and JNK1/2-deficient mice were detected (FIG. 26C). Similarly, no significant difference in serum transaminase activity between the control and JNK1/2-deficient mice was observed (FIG. 26D). The JNK1/2-deficient mice and control mice also exhibited similar mortality in response to the ConA challenge (FIG. 22B). Gene expression analysis in JNK1/2-deficient mice did not indicate a consistent loss of AP1-related gene expression, p53 pathway related gene expression, or Tnfα mRNA expression in the liver compared to control mice (FIG. 26E). Measurement of serum cytokines demonstrated that JNK1/2-deficiency in hepatocytes did not cause decreased expression of TNFα or other cytokines examined in ConA-treated mice (FIG. 27A).

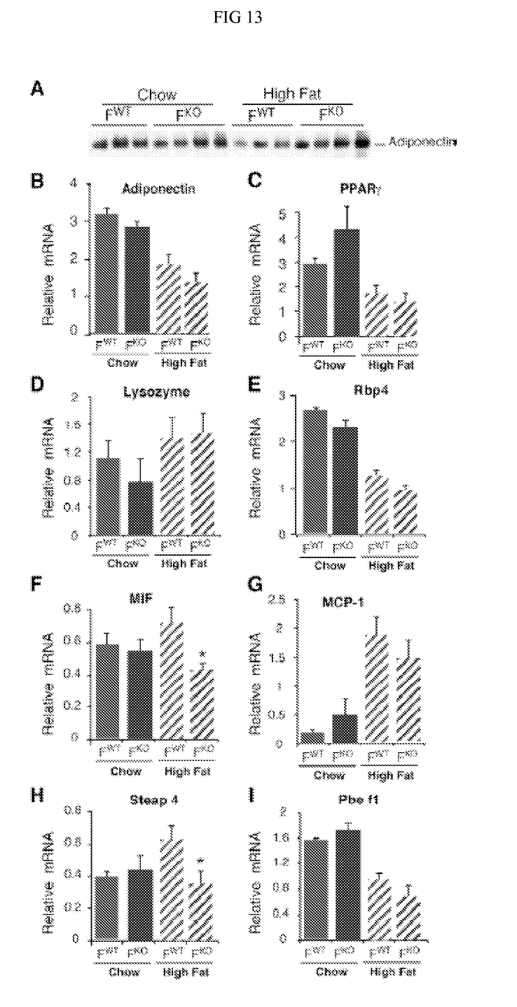

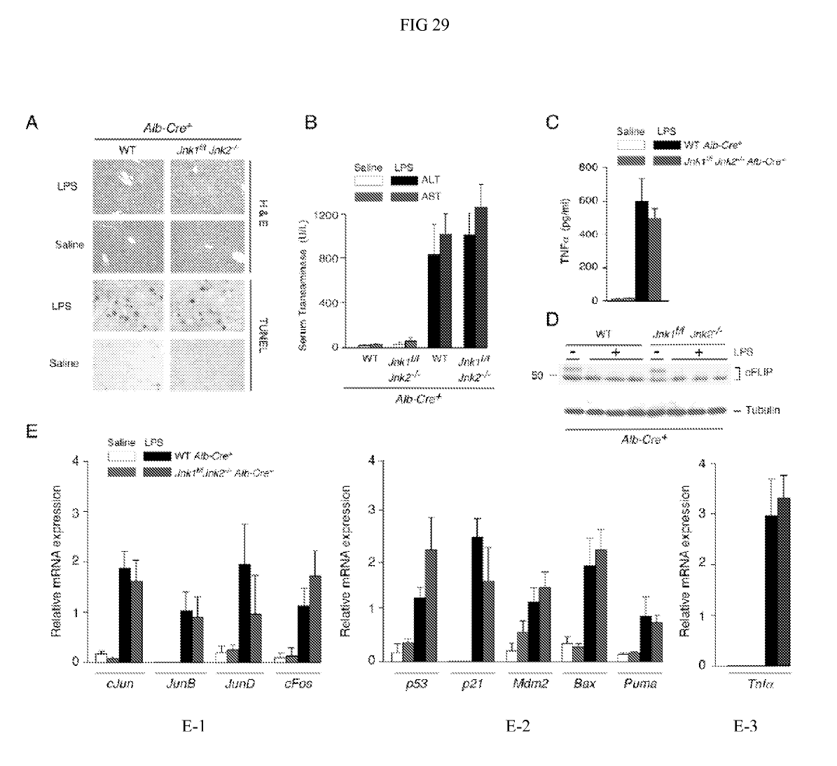

The analysis of ConA-treated mice demonstrated that JNK expression in hepatocytes is not required for the development of hepatitis (FIGS. 26, 18C & 22B). This conclusion was confirmed by studies of LPS-induced hepatitis (FIGS. 29 & 25A). LPS caused similar hepatic damage in control and hepatocyte-specific JNK1/2-deficient mice that was detected by histological analysis of liver sections (FIG. 29A), TUNEL labeling of apoptotic cells in liver sections (FIG. 13A), and serum transaminase activity (FIG. 29B). Furthermore, JNK1/2-deficiency in hepatocytes did not cause changes in the LPS-induced expression of AP1-related genes, p53 pathway-related genes, or Tnfα mRNA in liver (FIG. 29E). LPS also caused a similar increase in the serum concentration of TNFα in mice with JNK1/2-deficient hepatocytes and control mice (FIGS. 29C & 28A). Moreover, cFLIP expression in LPS-treated mice was not affected by hepatocyte-specific deficiency of JNK1/2 (FIG. 29D). These data demonstrate that there is no critical role of JNK in hepatocytes for the development of hepatitis, including treatment with ConA or LPS (FIGS. 26, 29 & 18 C,D). This finding contrasts with conclusions drawn from previous studies that have suggested an essential role of JNK in hepatocytes for ConA-induced hepatitis (Chang et al., 2006, supra; Kamata et al., Cell, 120, 649 (2005); Maeda et al., Immunity, 19, 725 (2003)).

### Example 5

### JNK is Required for TNFα Expression, but is not Required for TNFα-Induced Hepatic Damage

As described herein, the requirement of JNK for hepatocyte death during the development of hepatitis (FIGS. 21 & 24) is not mediated by the function of JNK in hepatocytes (FIGS. 26, 27, 29). These data suggest that JNK may play a non-cell autonomous essential role. One possibility is that there is a requirement of JNK for ConA- and LPS-induced expression of inflammatory cytokines by cells that are not hepatocytes. This is consistent with the finding that Mx1-Cre⁺JNK1/2-deficient mice, that are protected against ConA- and LPS-induced hepatitis (FIGS. 21, 18C,D & 24), exhibit defects in the expression of inflammatory cytokines, including TNFα (FIGS. 21D, 23 & 24D,E). In contrast, hepatocyte-specific JNK1/2-deficiency did not protect against hepatitis and did not cause marked changes in inflammatory cytokine expression (FIGS. 27A, 29C,E & 28A).

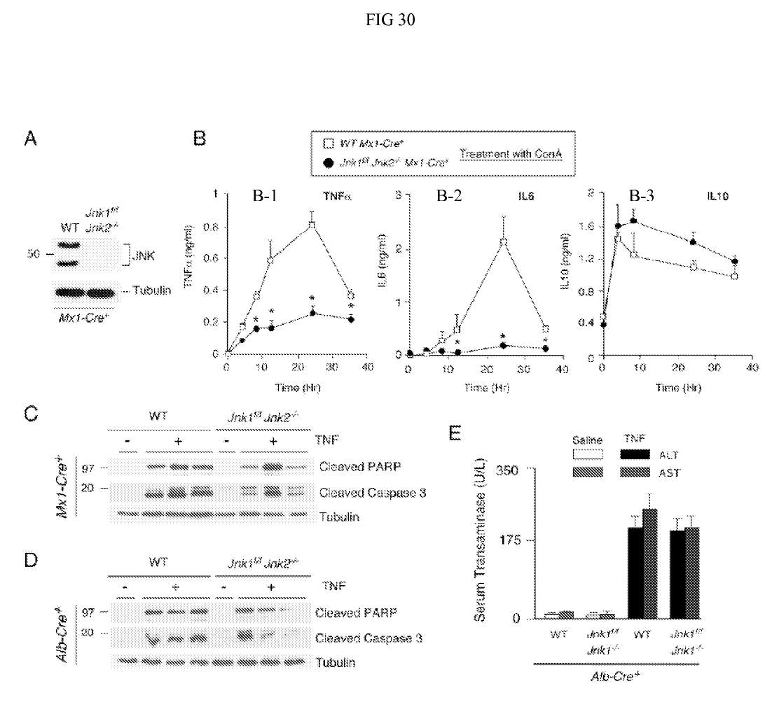

To test whether JNK is required for inflammatory cytokine expression, in vitro primary cultures of bone marrow-derived macrophages from polyIC-treated control (Mx1-Cre) and JNK1/2-deficient mice (Mx1-Cre⁺Jnk1^{f/f}Jnk2^{-/-}). No JNK was detected in the JNK1/2-deficient macrophages by immunoblot analysis (FIG. 30A). Treatment with ConA demonstrated increased concentrations of cytokines (TNFα and IL6) in the culture medium of control macrophages (FIG. 30B). The amount of these cytokines in the culture medium of JNK1/2-deficient macrophages was markedly suppressed (FIG. 30B). In contrast, JNK1/2-deficiency did not alter the expression of IL10 (FIG. 30B). Similarly, JNK1/2-deficient T cells exhibited defects in ConA-stimulated expression of TNFα (FIG. 20). Together, these data demonstrate that JNK1/2 is required for the expression of the inflammatory cytokine TNFα.

If the essential role of JNK in hepatitis (FIGS. 21 & 24) reflects a requirement of JNK for the expression of inflammatory cytokines (FIGS. 23, 30B & 20), it would be predicted that the defect in hepatitis caused by JNK-deficiency would be complemented by treatment of mice with TNFα. No significant difference were detected between control and JNK-deficient mice in TNFα-induced serum aminotransferase activity (FIG. 30E) and mortality (FIG. 25B). Immunoblot analysis demonstrated that TNFα caused similar caspase 3 activation and PARP cleavage in the liver of control mice, Mx1-Cre⁺JNK1/2-deficient mice, and Alb-Cre⁺JNK1/2-deficient mice (FIG. 30C,D). These data confirm that the essential role of JNK1/2 for hepatitis is mediated by a requirement of JNK1/2 for inflammatory cytokine expression, including TNFα, and does not reflect a role for JNK in TNFα-stimulated hepatocyte death.

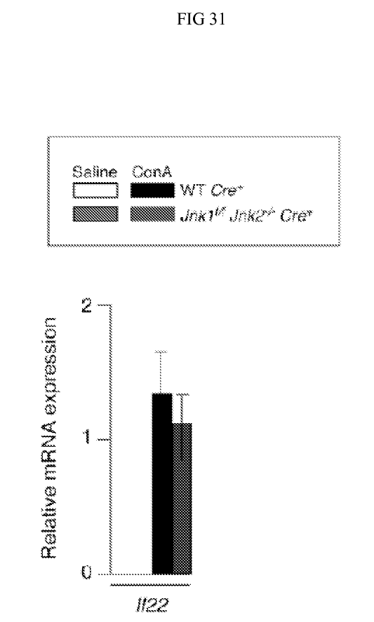

The pro-death role of inflammatory cytokines in hepatitis is opposed by factors that sustain cell survival. One example is the role of IL22 to activate the Stat3 and AKT signaling pathways in hepatocytes. Indeed, loss of IL22 expression increases ConA-induced hepatitis (Zenewicz et al., Immunity, 27, 647 (2007)). Altered expression of IL22 might therefore contribute to the effects of JNK1/2-deficiency on ConA-induced hepatitis. However, no significant difference in IL22 expression was detected between control and JNK1/2-deficient mice (FIG. 31). Together, these data indicate that IL22 does not mediate the effects of JNK1/2-deficiency on hepatitis.

### Example 6

### JNK is Required for TNF Expression by Hematopoietic Cells

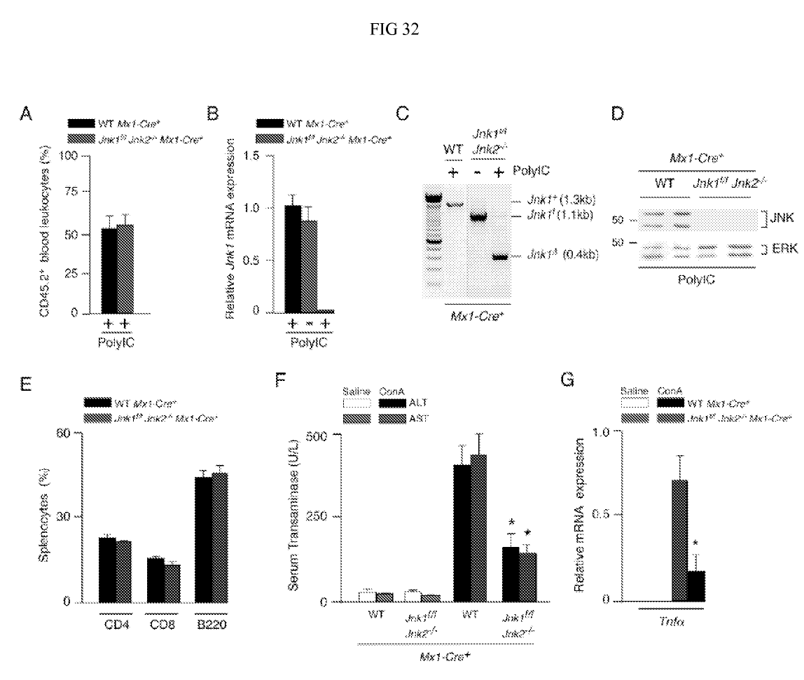

Hematopoietic cells represent one source of the inflammatory cytokines that may cause hepatitis following treatment with ConA or LPS (Dong et al., Cell. Mol. Immunol., 4, 241 (2007)). To test the role of hematopoietic cells, radiation chimeras were constructed by transplantation of control (Mx1-Cre⁺) and JNK1/2-deficient (Mx1-Cre⁺ Jnk1^{f/f}Jnk2^{-/-}) bone marrow from polyIC-treated donor mice into lethally irradiated congenic recipient mice. Efficient reconstitution of B6.SJL (CD45.1) mice with Jnk1^{+/+}Jnk2^{+/+} or Jnk1^{Δ/Δ}Jnk2^{-/-} bone marrow from C57BL/6J (CD45.2) mice was confirmed by staining peripheral blood leukocytes with antibodies to CD45.1/CD45.2 and analysis by flow cytometry. Indeed, competitive repopulation assays demonstrated that mice, reconstituted with an equal number of wild-type B6.SJL plus Jnk1^{Δ/Δ}Jnk2^{-/-} or Jnk1^{+/+}Jnk2^{+/+} C57BL/6J bone marrow cells, displayed similar numbers of CD45.1 and CD45.2 peripheral blood leukocytes at 2 months post-transplantation (FIG. 32A). Together, these data demonstrate that JNK1 and JNK2 are not essential for the re-population of the hematopoietic compartment following bone marrow transplantation.

Radiation chimeras with Jnk1^{+/+}Jnk2^{+/+} or Jnk1^{Δ/Δ}Jnk2^{-/-} hematopoietic cells in the blood were identified by quantitative RT-PCR analysis of Jnk1 mRNA expression (FIG. 32B), by PCR analysis of genomic DNA (FIG. 32C), and by immunoblot analysis using a JNK antibody (FIG. 32D). Flow cytometry analysis of splenocytes demonstrated similar numbers of CD4 T cells, CD8 T cells, and B cells in mice reconstituted with control and JNK1/2-deficient bone marrow (FIG. 32E).

Long-lived resident hematopoietic cells in the liver are slowly replaced following bone marrow transplantation; therefore mice were examined at 6 months post-transplantation (Alves-Guerra et al., J. Biol. Chem., 278, 42307 (2003)). Treatment of the radiation chimeras transplanted with Jnk1^{+/+}Jnk2^{+/+} bone marrow demonstrated that treatment with ConA caused hepatic damage, including increased amounts of serum transaminase activity (FIG. 32F). In contrast, radiation chimeras transplanted with Jnk1^{Δ/Δ}Jnk2^{-/-} bone marrow were protected against hepatic damage caused by ConA, including reduced amounts of serum transaminase activity (FIG. 32F), and decreased amounts of Tnfα mRNA in the liver and TNFα protein expression in blood (FIG. 32G). Together, these data confirm that JNK1/2 in hematopoietic cells is required for the development of hepatitis.

### Example 7

### Prevention of Steatosis by Hepatic JNK1

Non-alcoholic fatty liver disease is the leading cause of liver dysfunction in the non-alcoholic, viral hepatitis-negative, population in the USA and Europe. See Skelly et al., J. Hepatol. 35, 195 (2001); Angulo and Lindor, Best Pract Res Clin Gastroenterol 16, 797 (2002); and Cortez-Pinto et al., J Hepatol 44, 197 (2006). The disease represents a spectrum of liver pathologies, including steatosis, non-alcoholic steatohepatitis, and nonalcoholic cirrhosis. The incidence of non-alcoholic fatty liver disease is associated with obesity, dyslipidemia, insulin resistance, and diabetes (Anstee and Goldin, Int J Exp Pathol 87, 1 (2006)). It is likely that this disease represents one aspect of metabolic syndrome (Sanyal, Gastroenterology 123, 1705 (2002); Marchesini et al., Hepatology 37, 917 (2003)). The protein kinase JNK1 is implicated in the pathogenesis of metabolic syndrome because Jnk1−/− mice are protected against steatosis (Schattenberg et al., Hepatology 43, 163 (2006)). However, as described above, hepatic function, including steatosis and insulin resistance, can be regulated by JNK1 in adipose tissue. The role of hepatic JNK1 is therefore unclear.

To test the role of JNK1 in the liver, mice were created without (Alb-cre Jnk1^{+/+}; L^{WT}) and with (Alb-cre Jnk1^{LoxP/LoxP}; L^{KO}) a selective defect in the expression of JNK1 in hepatocytes. Measurement of JNK activity demonstrated that a high fat diet (HFD) caused JNK activation in the liver and adipose tissue of control (L^{WT}) mice, but JNK activation was detected only in adipose tissue and not the liver of L^{KO} mice. A JNK substrate site (Ser-307) that negatively regulates the insulin receptor substrate IRS-1 exhibited increased phosphorylation in the liver of HFD-fed L^{WT} mice, but not L^{KO} mice. Together, these data indicate that mice with hepatocyte specific JNK1-deficiency represent a model for the analysis of hepatic JNK1-deficiency.

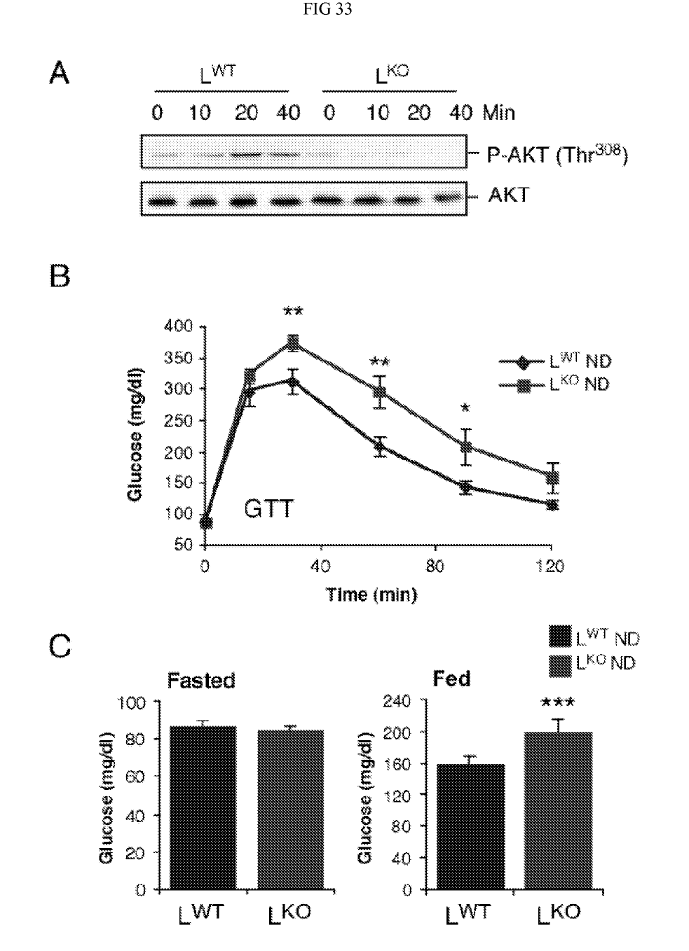

Studies using intravenous administration of adenovirus vectors to interfere with the JNK pathway in the liver suggest that hepatic JNK negatively regulates insulin signaling in the liver (Nakatani et al., J Biol Chem 279, 45803 (2004); Yang et al., J Biol Chem 282, 22765 (2007).). In contrast, in the experiments described herein, HFD-fed L^{KO} and L^{WT} mice exhibited similar glucose intolerance, glucose-induced insulin release, insulin-induced decrease in blood glucose concentration, and hyperglycemia. Furthermore, hyperinsulinemic-euglycemic clamp studies demonstrated a similar decrease in hepatic insulin action in HFD-fed L^{WT} and L^{KO} mice. These data indicated that JNK1-deficiency in hepatocytes does not protect against diet-induced insulin resistance. Moreover, it was found that chow-fed L^{KO} mice exhibited a profound defect in glucose-induced activation of hepatic AKT (FIG. 33A), glucose intolerance (FIG. 33B), and mild hyperglycemia (FIG. 33C).

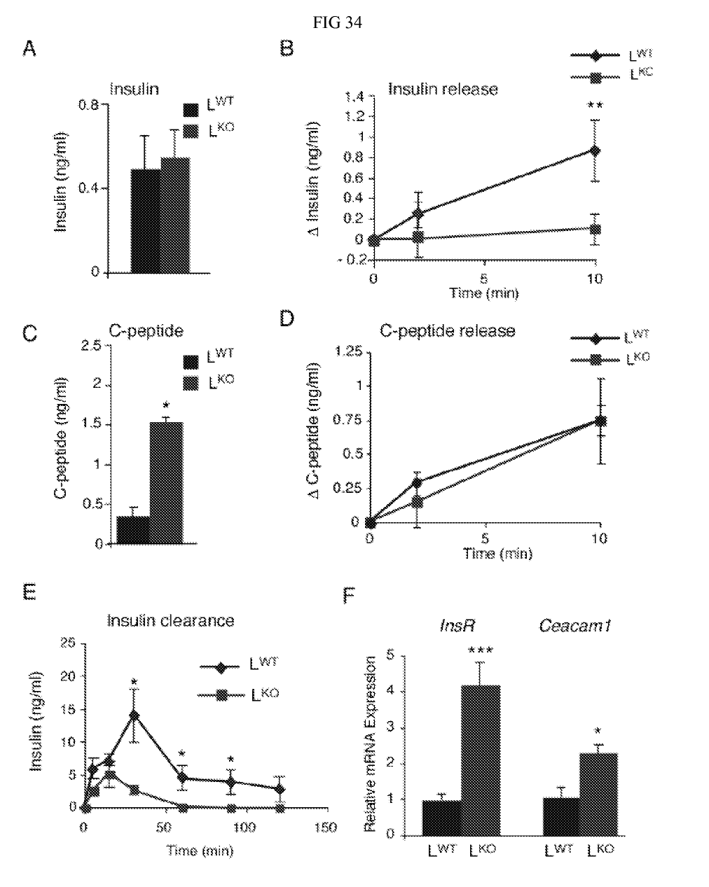

The major defect in glucose-induced hepatic insulin signaling observed in chow-fed L^{KO} mice (FIG. 33A) may reflect a reduction in the blood concentration of insulin. No significant difference in the fasting blood insulin concentration between L^{WT} and L^{KO} mice was detected (FIG. 34A) However, the amount of glucose-induced blood insulin was markedly decreased in L^{KO} mice compared with L^{WT} mice (FIG. 34B). This loss of blood insulin could result from decreased insulin secretion or increased insulin clearance. To distinguish between these possible mechanisms, the blood concentration of C-peptide (a proteolytic by-product of insulin processing) that is secreted together with insulin from β cells was examined. The C peptide concentration in the blood of L^{KO} mice was greatly increased compared with L^{WT} mice (FIG. 34CB). Nevertheless, a similar glucose-induced increase in blood C-peptide concentration was detected in L^{KO} and L^{WT} mice (FIG. 34D). Together, these observations suggest that insulin clearance was increased in L^{KO} mice compared with L^{WT} mice.

To test this hypothesis, mice were injected with human insulin and the time course of changes in the concentration of human insulin in the blood were measured. This analysis demonstrated that, compared with L^{WT} mice, the peak insulin concentration detected in L^{KO} mice was greatly reduced (FIG. 34 E). The clearance of blood insulin by L^{KO} mice was also markedly increased compared with L^{WT} mice (FIG. 34E). Together, these data indicate that the normal levels of blood insulin detected in fasting LKO mice are enabled by a compensatory increase in insulin secretion.

The liver is the major site of insulin clearance within the body. Indeed, it is estimated that 50% of insulin newly secreted by pancreatic β cells into the portal vein is internalized and degraded by the liver (Duckworth et al., Endocr. Rev. 19, 608 (1998)). Hepatic insulin clearance requires the insulin receptor (Michael et al., Mol Cell 6, 87 (2000)) and is regulated by Ceacam1 (Poy et al., Nat Genet. 30, 270 (2002)). The increased amounts of insulin receptor and Ceacam1 in the liver (FIG. 34F) may contribute to the increased insulin clearance in L^{KO} mice (FIG. 34F).

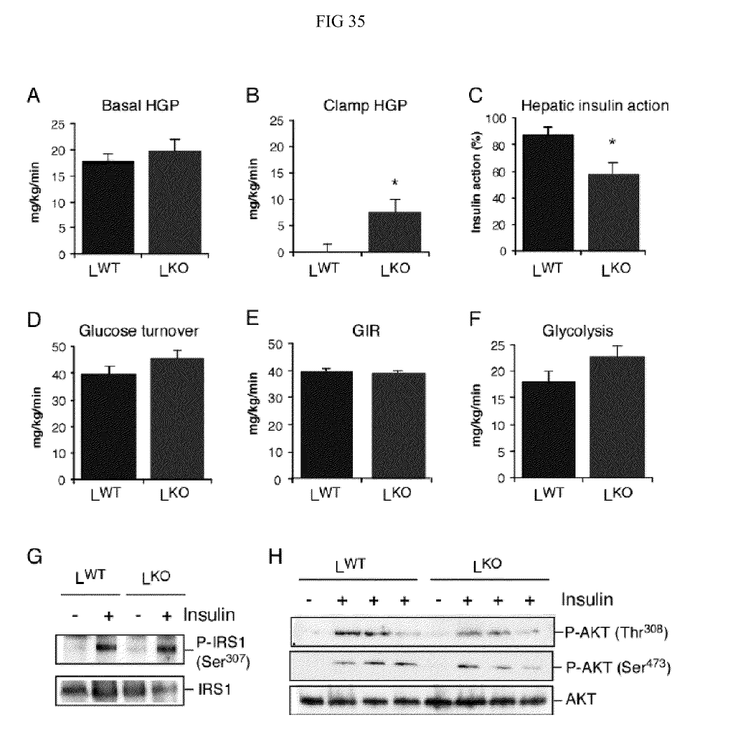

To further characterize the metabolic phenotype of chow-fed L^{KO} mice, a hyperinsulinemic-euglycemic clamp study was performed. This analysis demonstrated that L^{KO} mice exhibited increased hepatic glucose production during the clamp and decreased hepatic insulin action compared with L^{WT} mice (FIG. 35B,C). Indeed, the liver of L^{KO} mice expressed increased amounts of PGC-1α, a co-activator of the gluconeogenic gene transcription factors HNF4α and FOXO1. Basal hepatic glucose production and insulin-stimulated whole body glucose turnover were not altered in L^{KO} mice (FIGS. 35A & 35D). The fat mass and lean mass of L^{KO} and L^{WT} mice were similar (FIG. 35E,35F). Insulin treatment caused similar JNK independent negative feed-back phosphorylation of IRS1 on Ser-307 in L^{KO} mice compared with L^{WT} mice (FIG. 35G). However, decreased insulin-stimulated hepatic AKT activation was detected in L^{KO} mice compared with L^{WT} mice (FIG. 35H). Together, these data demonstrate that hepatic loss of JNK1 causes insulin resistance in liver.

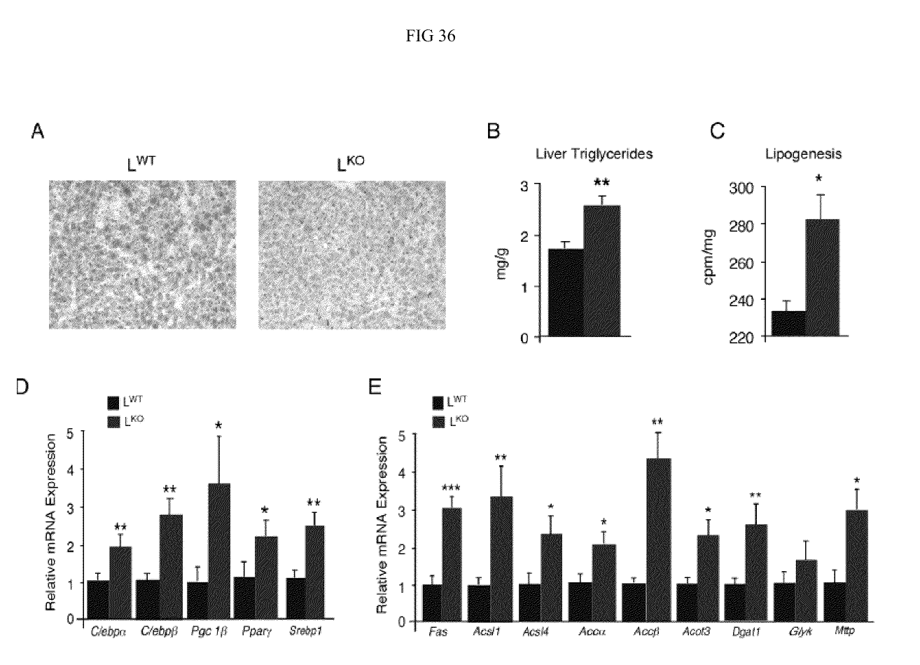

The increased insulin clearance and hepatic insulin resistance phenotype of chow-fed L^{KO} mice compared with L^{WT} mice (FIGS. 34 & 35) is likely to cause profound metabolic consequences. Indeed, it was found that chow-fed L^{KO} mice exhibited hepatic steatosis (FIG. 36A) associated with increased accumulation of triglyceride (FIG. 36B) and increased inflammation compared with L^{WT} mice.

The increased triglyceride accumulation in L^{KO} mice could be mediated by increased dietary lipid absorption, decreased fat oxidation, or increased lipogenesis. No differences were found between L^{KO} and L^{WT} mice in the respiratory exchange quotient [V_{CO2}]/[V_{O2}] or the intestinal absorption of dietary fat. The L^{KO} mice exhibited increased energy expenditure compared with L^{WT} mice, but no differences in food/water intake or physical activity in L^{KO} mice were detected. These observations do not support a role for increased dietary fat absorption or decreased fat oxidation as a cause of the hepatic steatosis in L^{KO} mice.

De novo lipogenesis may therefore contribute to steatosis in L^{KO} mice. Indeed, increased lipogenesis was detected in the liver of chow-fed L^{KO} mice compared with L^{WT} mice (FIG. 36C). Moreover, L^{KO} liver exhibited increased expression of genes that promote hepatic lipogenesis (C/ebpα, C/ebpβ, Pgc1β, Pparγ, and Srebp1) and also genes that encode enzymes that contribute to lipogenesis (Acacα/β, Acot3, Acsl1/4, Dgat1, Fas, and Gyk) and the export of triglyceride from the liver (Mttp) (FIG. 36D). These changes in gene expression contribute to hepatic steatosis in chow-fed L^{KO} mice compared with L^{WT} mice. Furthermore, the increased expression of C/ebpβ in the liver of L^{KO} mice may contribute to insulin clearance by increasing insulin receptor expression (FIG. 34D,E).

Steatosis is a widespread human disease that can progress to steatohepatitis and liver failure. The finding that the loss of JNK1 in hepatocytes causes steatosis identifies a possible complication of drug therapies involving JNK1 inhibition designed to treat metabolic syndrome. Nevertheless, steatosis is prevented in Jnk1−/− mice. This observation suggests that the metabolic milieu in response to JNK1-deficiency in different organs may compensate for the effects of JNK1-deficiency in hepatocytes. Indeed, JNK1 plays a major regulatory role in adipose tissue leading to protection against HFD-induced hepatic steatosis (see Example 2). This is consistent with the finding that treatment of mice with a JNK inhibitor can protect against the effects of feeding a HFD. However, not all of the deleterious effects of hepatocyte-specific JNK1-deficiency are compensated in Jnk1^{-/-} mice, including increased insulin clearance.

### Example 8

### Role of Muscle JNK1 in Obesity-Induced Insulin Resistance

As described herein, JNK1 is activated when mice are fed a HFD. Moreover, Jnk1^{-/-} mice are protected against HFD-induced insulin resistance. The mechanism of protection is mediated, in part, by the failure of Jnk1^{-/-} mice to develop HFD-induced obesity. However, JNK1 can regulate insulin resistance independently of obesity. Mice with adipose tissue-specific JNK1-deficiency develop normal diet-induced obesity, but exhibit selective protection against HFD-induced insulin resistance in both the liver and adipose tissue. These data indicate that adipose tissue JNK1 plays a critical role during the development of HFD-induced insulin resistance.

The liver plays a role in insulin-stimulated disposal of blood glucose during the postprandial state because of reduced gluconeogenesis and increased glycogen synthesis. However, glucose uptake by skeletal muscle also makes a major contribution to insulin-stimulated glucose disposal. Muscle may therefore be an important target of obesity-induced JNK1 signaling and the regulation of glucose homeostasis.

This example examines the effect of muscle-specific ablation of the Jnk1 gene in mice. It was found that HFD-fed control mice (Mck-Cre⁺Jnk1^{+/+}, M^{WT}) and muscle-specific JNK1-deficient mice (Mck-Cre⁺Jnk1^{LoxP/LoxP}, M^{KO}) became similarly obese. However, M^{KO} mice were selectively protected against HFD-induced insulin resistance. This analysis demonstrates that muscle JNK1 contributes to the effects of obesity on insulin resistance.

To test the role of JNK1 in muscle, mice without (M^{WT}) and with (M^{KO}) a selective defect in the expression of JNK1 in muscle were produced. Measurement of JNK activity demonstrated that a HFD caused JNK activation in muscle, liver and adipose tissue of control (M^{WT}) mice, but JNK activation was detected only in liver and adipose tissue of M^{KO} mice. Together, these data indicate that mice with muscle-specific JNK1-deficiency represent a model for the analysis of muscle JNK1-deficiency.

It was tested whether muscle-specific JNK1-deficiency might alter HFD-induced obesity. Comparison of chow-fed and HFD-fed M^{WT} and M^{KO} mice demonstrated that muscle-specific JNK1-deficiency caused no defect in HFD-induced weight gain. Indeed, measurement of lean mass and fat mass by ¹H-MRS demonstrated no significant differences between HFD-fed M^{KO} and M^{WT} mice. This analysis demonstrated that muscle JNK1-deficiency is not a major factor that contributes to the profound effect of whole body JNK1-deficiency to suppress HFD-induced obesity.

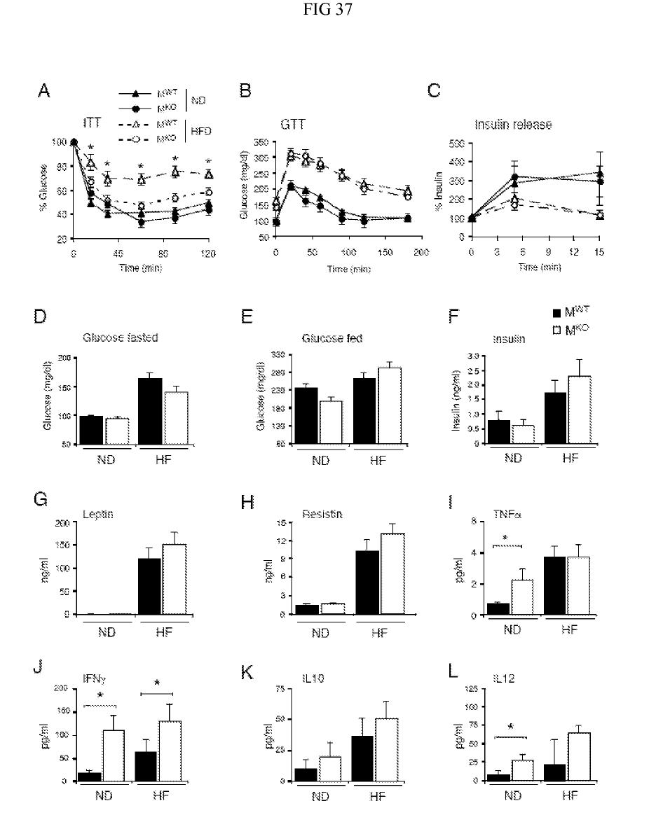

Feeding a HFD caused hyperglycemia and hyperinsulinemia in mice, but no significant differences between M^{KO} and M^{WT} mice were detected (FIG. 37D-F). Glucose tolerance tests (GTT) were performed to compare the response of M^{KO} and M^{WT} mice to a glucose challenge. It was found that the HFD caused glucose intolerance in both MKO and M^{WT} mice (FIG. 37B). The HFD-induced glucose intolerance was caused, in part, by decreased glucose-induced insulin release. No significant differences between M^{KO} and M^{WT} mice were found in studies of glucose induced insulin release (FIG. 37C). These data indicate that M^{KO} and M^{WT} mice mounted a similar response to a glucose challenge.

JNK1-deficiency in muscle did not affect the blood concentration of the adipokines leptin and resistin (FIG. 37G,H). However, analysis of the concentration of cytokines in the blood did indicate differences between M^{KO} and M^{WT} mice. Thus, the blood concentration of the inflammatory cytokines TNFα, IFN-γ, and IL12 was greater in M^{KO} mice than M^{WT} mice (FIG. 37I,J,L). In contrast, no significant difference in the concentration of the anti-inflammatory cytokine IL10 in the blood was detected between M^{KO} and M^{WT} mice (FIG. 37K).

The insulin receptor substrate IRS-1 can be negatively regulated by JNK-mediated phosphorylation of IRS-1 on Ser307 (Aguirre et al. J Biol Chem 275:9047-9054 (2000)). It was hypothesized that loss of JNK1 in muscle would attenuate negative regulatory phosphorylation of IRS-1 on Ser307 and increase insulin-stimulated tyrosine phosphorylation of IRS-1. To test this hypothesis, the effect of insulin treatment of M^{KO} and M^{WT} mice on insulin receptor and IRS-1 phosphorylation in muscle was examined. It was found that JNK1-deficiency did not affect insulin receptor tyrosine phosphorylation or the amount of expression of the insulin receptor or IRS-1. However, loss of JNK1 in muscle reduced inhibitory phosphorylation of IRS-1 on Ser307 and increased insulin-stimulated tyrosine phosphorylation of IRS-1. These data suggest that JNK1 plays an important role in the regulation of IRS-1 and therefore insulin signal transduction in muscle.

An insulin tolerance test (ITT) was performed to examine whether M^{KO} mice exhibit increased insulin sensitivity in vivo compared with M^{WT} mice (FIG. 37A). No significant differences between M^{KO} and M^{WT} mice were detected when these mice were fed a chow diet. In contrast, the HFD markedly suppressed the ITT response in M^{WT} mice, but HFD-fed M^{KO} mice remained insulin sensitive (FIG. 37A). These data suggest that M^{KO} mice exhibit protection against HFD-induced insulin resistance.

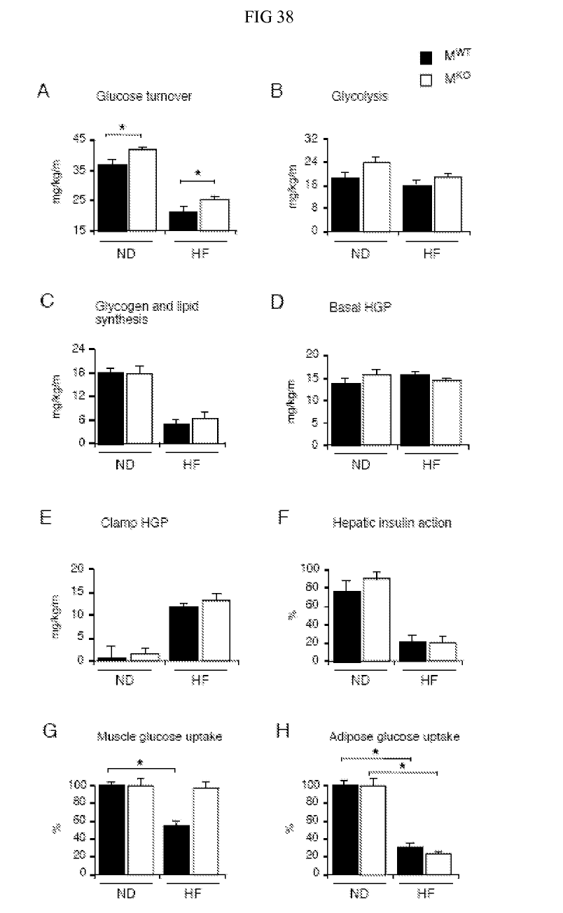

To confirm the conclusion that M^{KO} mice are more insulin sensitive, a hyperinsulinemic-euglycemic clamp study was performed in conscious mice following 4 weeks of HFD or chow diet. Insulin-stimulated whole body glucose turnover was modestly but significantly elevated in M^{KO} mice compared with M^{WT} mice following chow or HFD (FIG. 38A). Whole body glycolysis rates tended to be higher in chow-fed M^{KO} mice than chow-fed M^{WT} mice (P=0.057), but whole body glycogen plus lipid synthesis rates were not altered in M^{KO} mice (FIG. 38B,C). No statistically significant differences in basal or clamp glucose concentrations, hepatic glucose production (HGP) at basal or during insulin-stimulated (clamp) state, and hepatic insulin action were detected between M^{KO} and M^{WT} mice (FIG. 38D-F). Insulin-stimulated muscle glucose uptake was significantly reduced in HFD-fed M^{WT} mice compared with chow-fed M^{WT} mice, but muscle glucose uptake in HFD-fed M^{KO} mice was similar to chow-fed M^{KO} mice (FIG. 38G). In contrast, the HFD caused a similar decrease in insulin-stimulated glucose uptake by adipose tissue in M^{KO} and M^{WT} mice (FIG. 38H). These data demonstrate that M^{KO} mice exhibit a selective increase in skeletal muscle insulin sensitivity.

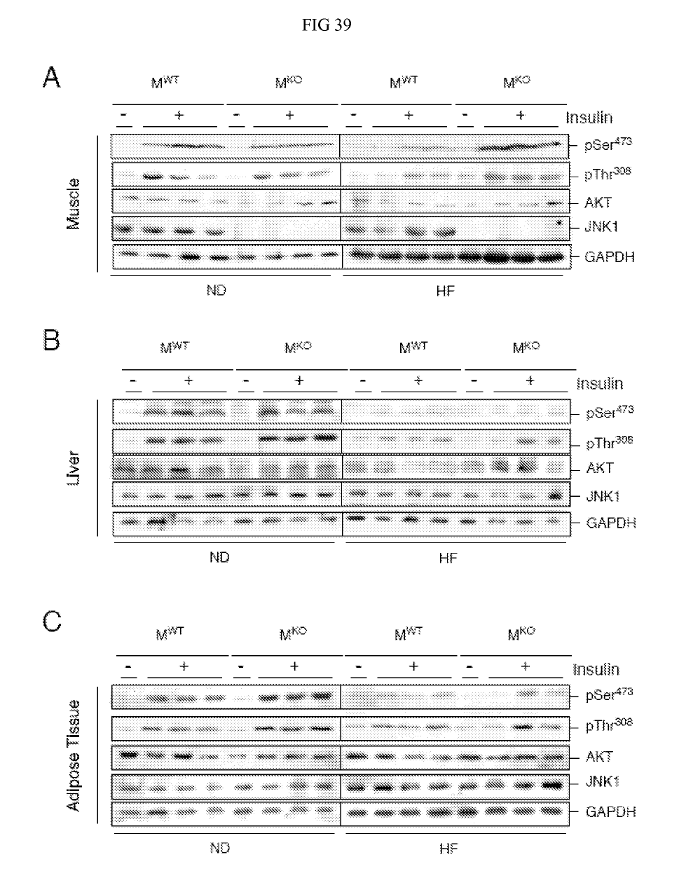

To obtain biochemical evidence for peripheral insulin sensitivity, insulin-stimulated AKT activation was examined in muscle, liver, and adipose tissue of M^{KO} and M^{WT} mice (FIG. 39). Insulin treatment of chow-fed M^{KO} and M^{WT} mice caused increased AKT activation. Feeding a HFD suppressed insulin-stimulated AKT activation in the liver and adipose tissue of both M^{KO} and M^{WT} mice (FIG. 39B,C). In contrast, the HFD suppressed insulin-stimulated AKT activation in muscle of M^{WT} mice, but not M^{KO} mice (FIG. 39A). These data support the conclusion that M^{KO} mice exhibit a selective rescue from HFD-induced skeletal muscle insulin resistance.

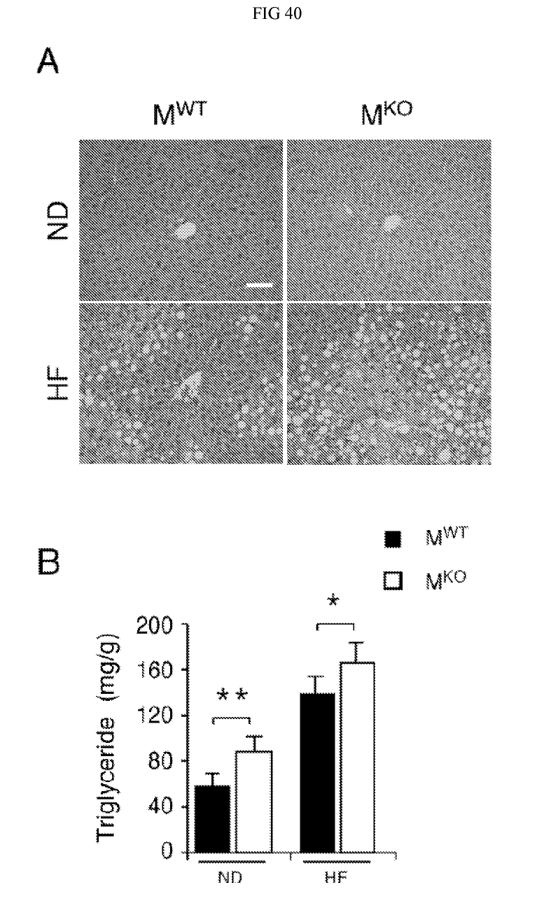

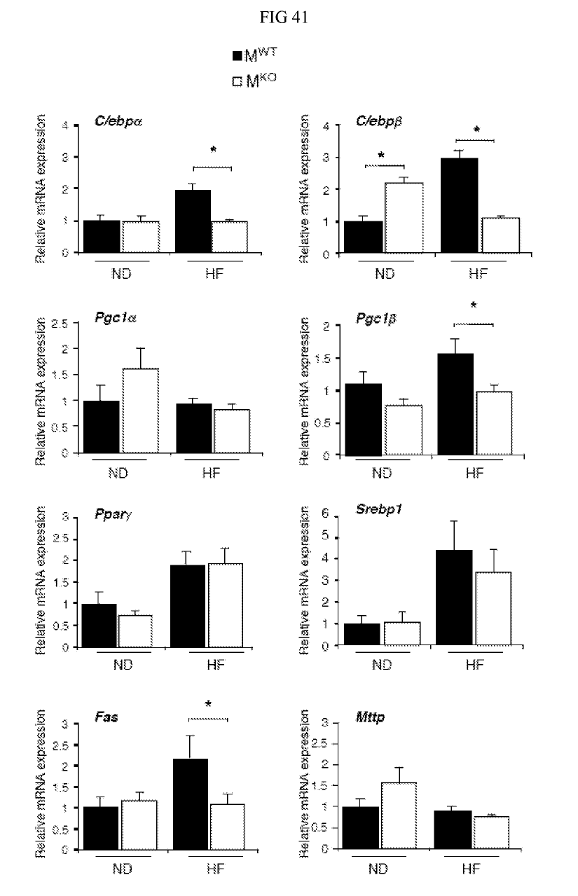

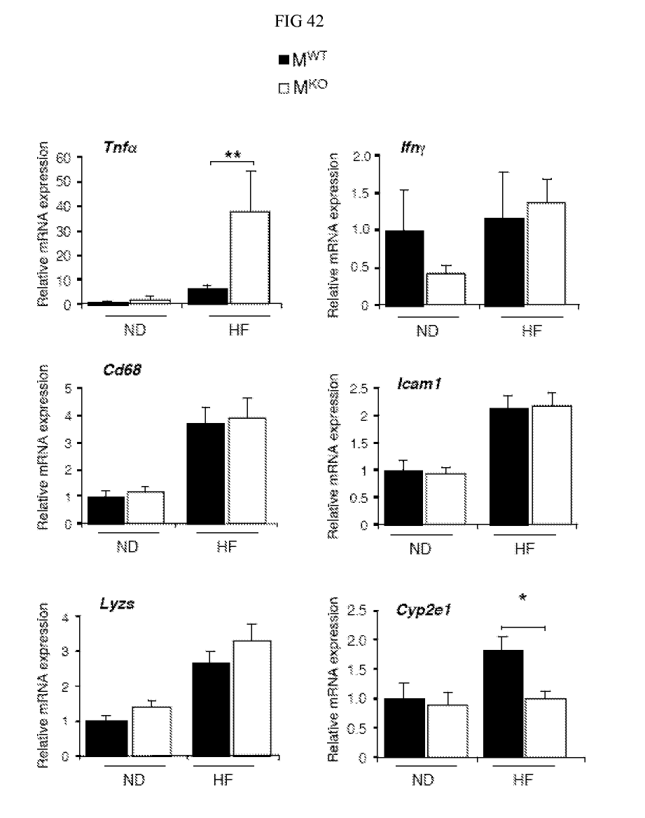

Ablation of the Jnk1 gene in muscle may lead to changes in other tissues. Indeed, comparison of the liver of M^{KO} and M^{WT} mice demonstrated that muscle JNK1-deficiency caused increased hepatic steatosis (FIG. 40A). Measurement of hepatic triglyceride accumulation demonstrated increased amounts of triglyceride in both chow-fed and HFD-fed M^{KO} mice compared with M^{WT} mice (FIG. 40B). The increased hepatic triglyceride accumulation was not accounted for by increased expression of a lipogenic transcription factor/coactivator (e.g. Srebp1, C/ebpα, C/ebpβ, Pgc1β) or lipogenic genes (e.g. Fas) (FIG. 41). However, the triglyceride accumulation in M^{KO} mice may account for increased expression of Tnfα and Cyp2e1 mRNA that was detected in the liver of M^{KO} mice compared with M^{WT} mice (FIG. 42).

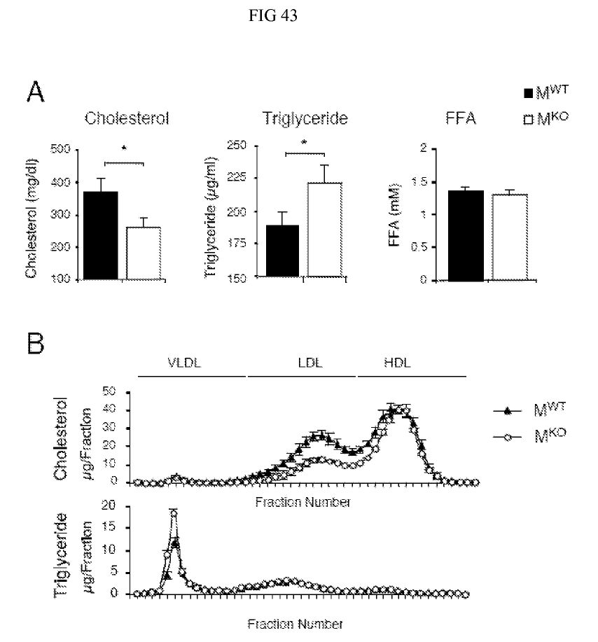

The increased accumulation of hepatic triglyceride was associated with increased amounts of triglyceride in the blood of M^{KO} mice compared with M^{WT} mice (FIG. 43A). Triglyceride in the liver is exported to the blood in the form of serum lipoprotein (VLDL). No significant difference in expression of the triglyceride transport protein Mttp mRNA in the liver of M^{KO} and M^{WT} mice was detected (FIG. 41). However, increased amounts of VLDL triglyceride and decreased amounts of LDL and HDL cholesterol were found in the blood of M^{KO} mice compared with M^{WT} mice (FIG. 43B). This increased amount of VLDL triglyceride might result from decreased triglyceride hydrolysis by lipoprotein lipase (LPL). Indeed, muscle LPL is a major contributor to VLDL triglyceride hydrolysis in vivo and muscle-specific Lpl knockout mice exhibit increased blood VLDL triglyceride and redistribution of triglyceride to non-muscle tissues within the body. Quantitative RT-PCR analysis demonstrated that Lpl mRNA expression in quadriceps muscle of M^{KO} mice was reduced by 60±16% compared with M^{WT} mice (mean±SE; n=5; P<0.05), but no significant difference in Lpl mRNA expression in adipose tissue of M^{KO} and M^{WT} mice was detected (n=5; P>0.05). This decrease in muscle LPL expression may contribute to the increased amount of triglyceride detected in the blood and the liver of M^{KO} mice.

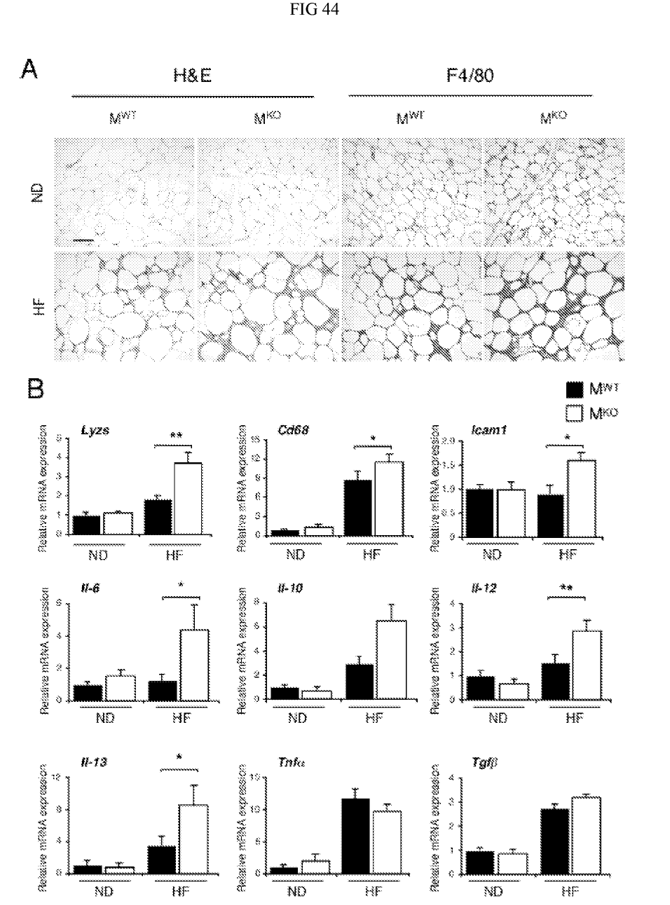

It is likely that increased triglyceride in the blood of M^{KO} mice may affect other tissues. To test this hypothesis, adipose tissue of M^{KO} and M^{WT} mice was compared. This analysis demonstrated that muscle-specific JNK1-deficiency increased the HFD-induced infiltration of adipose tissue by myeloid cells (FIG. 44). Morphological analysis and immunohistochemical analysis of adipose tissue sections demonstrated increased numbers of F4/80-positive myeloid cells in HFD-fed M^{KO} mice compared with M^{WT} mice (FIG. 44A). Quantitative RT-PCR analysis of gene expression confirmed increased expression of the myeloid marker genes Lyzs and Cd68 (FIG. 44B). Increased expression of Icam1 and the cytokines Il-6, Il-12, and Il-13 (but not Il-10, Tnfα, or Tgfβ1) was also detected in the adipose tissue of M^{KO} mice compared with M^{WT} mice (FIG. 44B). It is established that IL-13 expressed by adipocytes plays a key role in the activation of macrophages by the alternate M2a pathway (mediated by PPARγ/δ) that influences insulin sensitivity. Together, these data indicate that muscle-specific JNK1-deficiency caused an increased inflammatory response in adipose tissue. It is possible that this response is mediated by the increased amount of triglyceride in the blood of M^{KO} mice compared with M^{WT} mice.

HFD-fed mice with selective deficiency of JNK1 in adipose tissue or muscle exhibit improved insulin sensitivity compared with control mice. These data support the conclusion that JNK1 is important for the normal development of HFD-induced insulin resistance. JNK1-dependent cytokine expression can contribute to inflammation-associated insulin resistance in HFD-fed mice. This mechanism allows JNK1 in one tissue to regulate insulin resistance in other tissues; for example JNK1-dependent IL6 expression by adipose tissue can mediate hepatic insulin resistance.

JNK1 may also function by a more direct mechanism by inhibiting insulin signal transduction. One example is represented by phosphorylation of the adapter protein IRS1 on the negative regulatory site Ser307 that prevents the interaction of IRS1 with the insulin receptor. This mechanism may be important for the improved insulin sensitivity of adipose tissue in HFD-fed mice with adipose-specific JNK1-deficiency. This conclusion is consistent with the finding that Jnk1 gene ablation in adipose tissue suppressed IRS1 phosphorylation on Ser307. Similarly, muscle-specific JNK1-deficiency decreased IRS1 phosphorylation on Ser307 and improved insulin sensitivity (FIGS. 38 & 39). Indeed, muscle-specific JNK1-deficiency protected insulin-stimulated AKT activation selectively in muscle, but not liver or adipose tissue, after feeding a HFD (FIG. 39A). This observation suggests that muscle JNK1 can regulate insulin resistance by a cell autonomous mechanism that involves, at least in part, negative regulatory phosphorylation of IRS1.

One unexpected consequence of muscle-specific JNK1-deficiency was the finding that the blood triglyceride concentration in M^{KO} mice was greater than M^{WT} mice (FIG. 43). The mechanism that accounts for the increase in blood triglyceride is unclear. However, reduced expression of muscle LPL may represent one contributing factor. Reduced muscle LPL expression in M^{KO} mice may contribute to increased triglyceride accumulation in blood and liver (FIGS. 40 & 43) and increased inflammation of adipose tissue (FIG. 44), particularly in HFD-fed mice.

### Example 9

### Role of the Hypothalamic-Pituitary-Thyroid Axis in Metabolic Regulation by JNK1

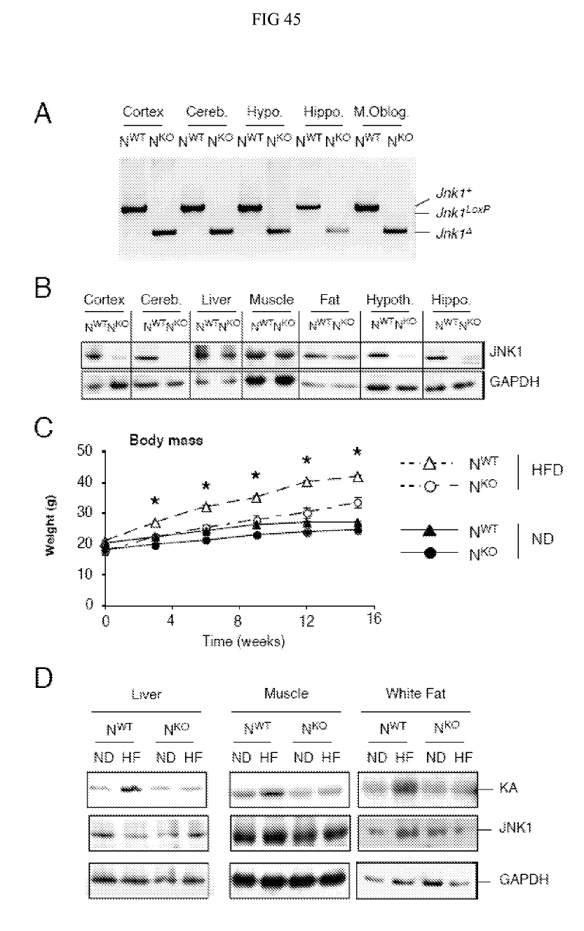

To investigate the role of JNK1 in the brain, compound mutant mice (Nestin-cre Jnk1^{LoxP/LoxP}) were created with a selective defect in the expression of JNK1. Genotype analysis of control (N^{WT}) and JNK1-deficient (N^{KO}) mice demonstrated that the Jnk1^{LoxP} allele was efficiently deleted in the nervous system of N^{KO} mice (FIG. 45A). Thus, the Jnk1 gene was ablated in all regions of the central nervous system of NKO mice that we examined, including the cortex, cerebellum, hypothalamus, hippocampus, and medulla oblongata (FIG. 45A). Immunoblot analysis demonstrated markedly reduced JNK1 protein in these sub-regions of the brain and normal amounts of JNK1 in liver, muscle, and adipose tissue (FIG. 45B). Control studies demonstrated that the Jnk1 gene was not deleted in β cells of the Islets of Langerhans of N^{KO} mice. These data indicate that N^{KO} mice exhibit a tissue-specific defect in JNK1 expression. N^{KO} mice therefore represent a model for the analysis of nervous system-specific JNK1-deficiency.

As described herein, selective JNK1-deficiency in adipose tissue, liver, muscle or myeloid cells caused no defect in HFD-induced obesity. These findings indicate that JNK1 function in another organ accounts for the effects of whole body JNK1-deficiency to suppress HFD-induced weight gain. Therefore it was tested whether nervous system-specific JNK1-deficiency might prevent HFD-induced weight gain. Comparison of chow-fed and HFD-fed N^{WT} and N^{KO} mice demonstrated that nervous system-specific JNK1-deficiency markedly reduced weight gain caused by a HFD (FIG. 45C).

The resistance to weight gain in HFD-fed N^{KO} mice may account for the finding that HFD-induced JNK activation in adipose tissue, muscle, and liver of N^{WT} mice was not detected in N^{KO} mice (FIG. 45D).

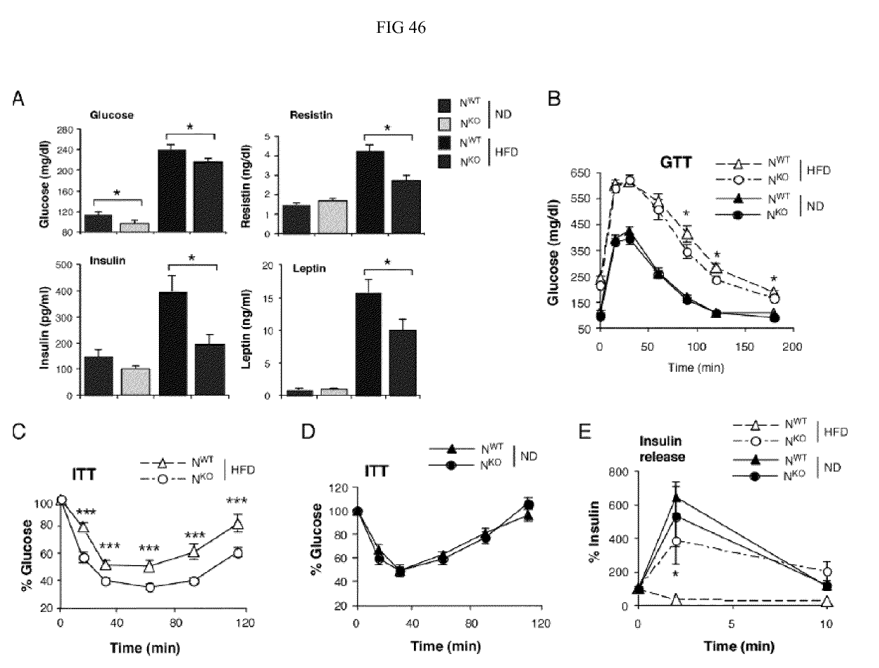

The hyperglycemia and hyperinsulinemia caused by feeding a HFD to N^{WT} mice was significantly reduced in HFD-fed N^{KO} mice (FIG. 46A). Similarly, the HFD-induced increase in the blood concentration of leptin was markedly attenuated in HFD-fed N^{KO} mice (FIG. 46A). Consistent with these observations, HFD-fed N^{KO} mice were more glucose tolerant (FIG. 46B), more responsive in an insulin tolerance test (FIG. 46C), and exhibited increased glucose-induced insulin release (FIG. 46E) compared with HFD-fed N^{WT} mice. These data indicate that HFD-fed N^{KO} mice show increased insulin sensitivity and improved β-cell function compared with HFD-fed N^{WT} mice. To confirm this conclusion, a hyperinsulinemic-euglycemic clamp study was conducted in conscious mice. This analysis demonstrated significant increases in steady-state glucose infusion rate, insulin-stimulated whole body glucose turnover, glycogen plus lipid synthesis, and hepatic insulin action in HFD-fed N^{KO} mice compared with HFD-fed N^{WT} mice. These data confirmed that HFD-fed N^{KO} mice are more insulin sensitive than HFD-fed N^{WT} mice.

To obtain biochemical evidence for insulin sensitivity, insulin-stimulated AKT activation was examined in adipose tissue, muscle, and liver of N^{KO} and N^{WT} mice. Insulin treatment increased AKT activation in chow-fed N^{KO} and N^{WT} mice. Insulin-stimulated AKT activation was suppressed in adipose tissue, muscle, and liver of HFD-fed N^{WT} mice, demonstrating insulin resistance in these organs. In contrast, studies of N^{KO} mice demonstrated that the HFD did not inhibit insulin-stimulated AKT activation in adipose tissue and muscle, and only partially suppressed AKT activation in liver. These data support the conclusion that N^{KO} mice exhibit protection against HFD-induced insulin resistance. This finding is consistent with the observation that N^{KO} mice did not gain weight in response to feeding a HFD (FIG. 45C).

The mechanism by which nervous system-specific JNK1-deficiency prevented HFD-induced weight gain was investigated. Examination of organ mass at necropsy indicated a significant reduction in the weight of epididymal white fat, intrascapular brown fat, quadriceps muscle, and liver in HFD-fed N^{KO} mice compared with N^{WT} mice. In contrast, no significant difference in heart mass was detected between N^{KO} and N^{WT} mice. Measurement of lean and fat mass using ¹H-MRS indicated that while reduced fat accumulation by N^{KO} mice was detected, the N^{KO} mice also exhibited reduced lean mass compared with N^{WT} mice. These data suggest that the defect in HFD-induced weight gain observed in N^{KO} mice was due to a reduction in both fat and lean body mass.

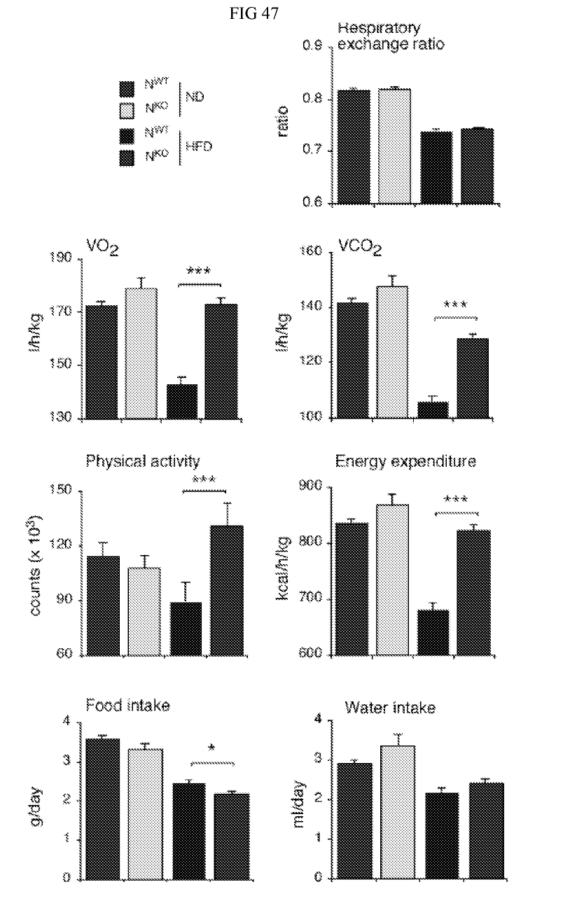

Metabolic cage analysis of N^{KO} and N^{WT} mice was performed to determine the effects of nervous system-specific JNK1-deficiency on energy balance (FIG. 47). The N^{KO} and N^{WT} mice were found to drink a similar volume of water and no difference in the respiratory quotient ([V_{CO2}]/[V_{O2}]) was detected. However, the HFD-fed N^{KO} mice exhibited increased physical activity and energy expenditure compared with HFD-fed N^{WT} mice (FIG. 47). A small decrease in food intake by HFD-fed N^{KO} mice compared with N^{WT} mice was also detected (FIG. 47). These changes in food intake, physical activity, and energy expenditure are consistent with the failure of HFD-fed N^{KO} mice to gain weight.

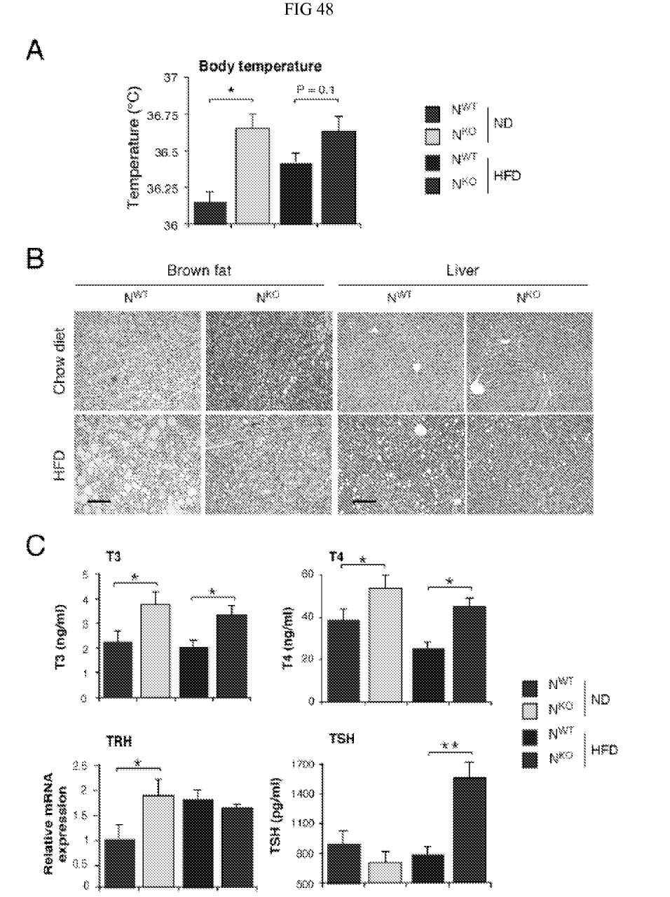

It was found that N^{KO} mice exhibited elevated body temperature (FIG. 48A) that was associated with a reduction in lipid accumulation by brown fat and liver in HFD-fed N^{KO} mice compared with HFD-fed N^{WT} mice (FIG. 48B). Gene expression analysis demonstrated that N^{KO} mice expressed larger amounts of mRNA derived from thyroid hormone target genes (Obregon, Thyroid 18: 185-195 (2008)). These data indicate that the thyroid hormone pathway is activated in N^{KO} mice. Indeed, increased levels of T4 and T3 were detected in the blood of N^{KO} mice compared with N^{WT} mice (FIG. 48C). This change was associated with increased expression of thyrotropin releasing hormone (Trh) mRNA in the hypothalamus of chow-fed N^{KO} mice, increased expression of thyroid stimulating hormone (Tsh) mRNA in N^{KO}N^{KO} the pituitary gland, and increased TSH protein in the blood of HFD-fed N^{KO} mice (FIG. 48C). TSH and TRH expression are subject to acute negative feed-back regulation by thyroid hormone. The presence of high levels of T4 and T3 in the blood of N^{KO} mice under conditions where TSH and TRH expression are elevated suggests that brain JNK1-deficiency disrupts the normal negative feed-back regulation of the hypothalamus-pituitary axis.

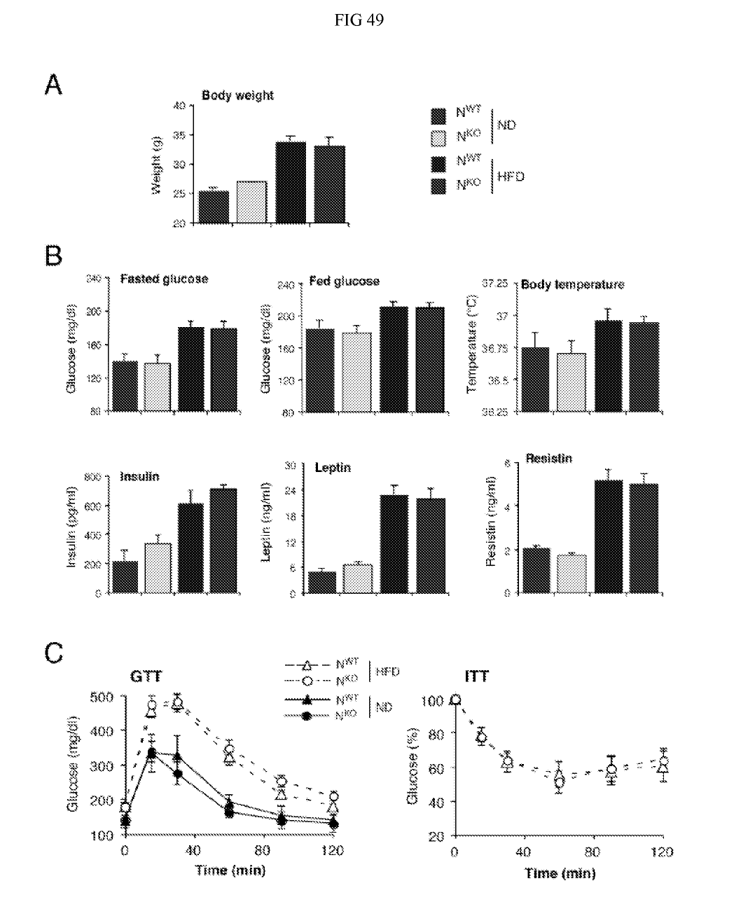

To test whether increased thyroid hormone signaling was causally related to the defect in HFD-induced weight gain in N^{KO} mice, the effect of treatment of mice with propylthiouracil (PTU), a drug that inhibits thyroperoxidase and prevents T4 production by the thyroid gland (Bjorkman and Ekholm, Biochemistry of thyroid hormone formation and secretion in The Thyroid Gland (ed. M. A. Greer), pp. 83-125. Raven Press, New York. 2000) was examined. N^{KO} and N^{WT} mice were treated with PTU in the drinking water and the effect of feeding a chow diet or a HFD was examined. Analysis of intrascapular brown fat demonstrated that PTU-treatment suppressed the increased expression of thyroid hormone responsive genes in NKO mice. These data demonstrate that PTU-treatment represents an effective model to study the role of thyroid hormone signaling in N^{KO} and N^{WT} mice. It was found that the PTU-treated N^{KO} and N^{WT} mice showed similar increases in body weight when fed a HFD (FIG. 49A). No significant differences in glucose, insulin and adipokine (leptin and resistin) concentrations in the blood or body temperature between PTU-treated N^{KO} and N^{WT} mice were detected (FIG. 49B). Similarly, no significant differences between PTU-treated N^{KO} and N^{WT} mice were detected in glucose and insulin tolerance tests (FIG. 49C). Together, these data demonstrate that inhibition of thyroid hormone by PTU-treatment markedly suppressed the metabolic phenotypes of NKO mice. This analysis supports the conclusion that increased thyroid hormone contributes to the metabolic phenotype of NKO mice.

To test the contribution of thyroid hormone to the phenotype of whole body JNK1 knockout mice, the effect of PTU-treatment of Jnk1−/− mice was examined. It was found that PTU-treatment significantly suppressed the effect of whole body JNK1-deficiency on HFD-induced weight gain, hyperglycemia, glucose intolerance, insulin sensitivity, and glucose-induced insulin release. However, the PTU-treatment caused greater suppression of the metabolic phenotype of NKO mice than Jnk1−/− mice, consistent with metabolic roles of JNK1 in both neuronal and non-neuronal tissues.

Studies of control mice demonstrate that HFD-induced obesity is mediated, in part, by reduced physical activity and energy expenditure (FIG. 47). In contrast, feeding a HFD to mice with JNK1-deficiency in the nervous system (N^{KO} mice) does not cause decreased physical activity and energy expenditure (FIG. 47). This maintenance of physical activity and energy expenditure in HFD-fed N^{KO} mice contributes to the failure of these mice to gain weight when fed a HFD. The increased energy expenditure in N^{KO} mice is mediated by activation of the hypothalamus-pituitary-thyroid axis. This conclusion is based upon several lines of evidence, including increased body temperature, increased expression of thyroid hormone-induced genes, and increased amounts of T4 and T3 in the blood of N^{KO} mice compared with N^{WT} mice. Moreover, pharmacological inhibition of thyroid hormone production abolished the metabolic phenotypes of N^{KO} mice, including marked suppression of HFD-induced weight gain. These data identify the hypothalamus-pituitary-thyroid axis as an important target of the metabolic actions of JNK1.

The thyroid hormone pathway is negatively regulated by JNK1. The increased amount of T4 and T3 in the blood of N^{KO} mice compared with N^{WT} mice correlates with increased expression of hypothalamic TRH and pituitary gland TSH (FIG. 48C). These changes in TRH and TSH expression were unexpected because thyroid hormone exerts powerful negative feed-back control of TRH and TSH expression. The association of increased T4 and T3 in the blood with increased expression of TRH and TSH in N^{KO} mice indicates that JNK1-deficiency in the brain disrupts the normal negative feed-back control of the hypothalamic-pituitary-thyroid axis.

In conclusion, this example demonstrates that JNK1-deficiency in the nervous system is sufficient to account for the role of JNK1 in the regulation of HFD-induced weight gain.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of monitoring JNK inhibition in a subject being treated with a JNK inhibitor, said method comprising:
a) obtaining a biological sample from a subject being treated with a JNK inhibitor;
b) determining the level of interleukin 6 (IL6) or tumor necrosis factor alpha (TNFα) in the biological sample; and
c) assessing a level of JNK inhibition based on the level of IL6 or TNFα in the biological sample.

2. The method of claim 1, wherein the JNK inhibitor is an anthrapyrazolone compound, a peptide, an antisense oligonucleotide, or an siRNA.

3. The method of claim 1, the method further comprising comparing the level of IL6 or TNFα in the biological sample to a control level of IL6 or TNFα, wherein a decrease in the level of IL6 or TNFα in the subject relative to that of the control level is indicative of a positive response to the therapy in the subject.

4. The method of claim 3, wherein the control level is the level of IL6 or TNFα in the subject before treatment with the JNK inhibitor.

5. The method of claim 3, wherein the control level is the level of IL6 or TNFα in a control population.

6. The method of claim 1, wherein the biological sample comprises one or more of whole blood, plasma, serum, and adipose tissue.

7. The method of claim 1, wherein an expression level of an mRNA encoding TNFα is measured in the biological sample.

8. The method of claim 1, wherein the level of IL6 or TNFα is determined immunologically.

9. The method of claim 8, wherein the level of IL6 or TNFα is determined using a monoclonal antibody.

10. The method of claim 9, wherein said monoclonal antibody is attached to a solid substrate.

11. The method of claim 10, wherein the solid substrate comprises a bead or a microtiter plate.

12. A method of identifying a JNK inhibitor, said method comprising:
a) contacting adipocytes in a culture medium with a test compound; and
b) monitoring expression of IL6 in the adipocytes, wherein the test compound is identified as JNK inhibitor if the expression of IL6 in the presence of the test compound is decreased relative to the expression of IL6 in the absence of the test compound.

13. The method of claim 12, wherein expression of IL6 is monitored by determining a level of IL6 protein in the culture medium.

14. The method of claim 12, wherein expression of IL6 is monitored by determining a level of mRNA encoding IL6 in the adipocytes.

15. An article of manufacture for monitoring treatment with a JNK inhibitor, the article of manufacture comprising reagents for determining the level of IL6 in a biological sample from the patient, wherein the reagents are attached to a solid phase.

16. The article of manufacture of claim 15, further comprising a reagent for measuring the level of tumor necrosis factor alpha (TNFα), interferon gamma (IFNγ), or macrophage migration inhibiting factor-1 (MIF-1).

17. A composition comprising a JNK inhibitor linked to a monoclonal antibody having binding affinity for an epitope on an adipocyte.

18. The composition of claim 17, wherein said JNK inhibitor is an anthrapyrazolone compound or a peptide.

19. A method for treating a patient having type 2 diabetes, the method comprising:
a) administering to the patient an amount of a JNK inhibitor effective to increase insulin sensitivity in the patient; and
b) monitoring IL6 levels in the serum of the patient to determine the efficacy of the treatment.

20. The method of claim 19, further comprising adjusting the amount of the JNK inhibitor administered to the patient based on the monitoring.

21. The method of claim 19, wherein the JNK inhibitor is targeted to adipose tissue.

22. An article of manufacture for monitoring treatment with a JNK inhibitor, said article of manufacture comprising reagents for determining the level of TNFα in a biological sample from the patient, wherein the reagents are attached to a solid phase.

23. The article of manufacture of claim 22, further comprising a reagent for measuring the level of interferon gamma (IFNγ), interleukin 2 (IL2), interleukin 4 (IL4), or IL6.

24. A method for treating a patient having hepatitis, the method comprising administering to the patient an amount of a JNK inhibitor effective to increase liver function in the patient, and monitoring TNFα levels in the serum of the patient to determine the efficacy of the treatment.

25. The method of claim 24, further comprising adjusting the amount of the JNK inhibitor administered to the patient based on the monitoring.

26. A method for treating a patient having type 2 diabetes, the method comprising administering to the patient an amount of a JNK inhibitor effective to increase insulin sensitivity in the patient, wherein said JNK inhibitor is targeted to adipose tissue.

27. The method of claim 26, said method further comprising monitoring IL6 levels in the serum of the patient to determine the efficacy of the treatment.

28. The method of claim 26, wherein the JNK inhibitor is conjugated to a monoclonal antibody having binding affinity for an epitope on an adipocyte.

29. A method for treating a patient having a disorder treatable with a JNK inhibitor, the method comprising
(a) administering to the patient an amount of the JNK inhibitor;
(b) determining the level of JNK inhibition based on the level of IL6 or TNFα in a biological sample from the patient; and
(c) administering an amount of JNK inhibitor different from (a) if the level of IL6 or TNFα determined in (b) indicates that more or less JNK inhibition in the patient is required.

30. The method of claim 29, wherein the disorder is type 2 diabetes.

31. The method of claim 29, wherein the disorder is hepatitis.


SEQUENCE LISTING (21 sequences)

SEQ ID NO: 1 - DNA, 24 bp, Artificial Sequence
  Primer
cctcaggaagaaagggcttatttc

SEQ ID NO: 2 - DNA, 24 bp, Artificial Sequence
  Primer
gaaccactgttccaatttccatcc

SEQ ID NO: 3 - DNA, 20 bp, Artificial Sequence
  Antisense Oligonucleotide
ctctctgtaggcccgcttgg

SEQ ID NO: 4 - DNA, 20 bp, Artificial Sequence
  Antisense Oligonucleotide
ctcatgatggcaagcaatta

SEQ ID NO: 5 - DNA, 42 bp, Artificial Sequence
  RNAi oligonucleotide with 2′-deoxythymidines to enhance nuclease resistance
cguggauuuauggucugugttttgcaccuaaauaccagacac

SEQ ID NO: 6 - DNA, 21 bp, Artificial Sequence
  Primer
tgtggggataaagcatcaggc

SEQ ID NO: 7 - DNA, 22 bp, Artificial Sequence
  Primer
ccggcagttaagatcacaccta

SEQ ID NO: 8 - DNA, 24 bp, Artificial Sequence
  Primer
tacatgcatacctactgcctgcct

SEQ ID NO: 9 - DNA, 24 bp, Artificial Sequence
  Primer
ttgggccagaagttcccttaggat

SEQ ID NO: 10 - DNA, 21 bp, Artificial Sequence
  Primer
gatgtgcgaactggacaccag

SEQ ID NO: 11 - DNA, 19 bp, Artificial Sequence
  Primer
catagggggcgtcaaacag

SEQ ID NO: 12 - DNA, 22 bp, Artificial Sequence
  Primer
ctggacaacatactgctaaccg

SEQ ID NO: 13 - DNA, 22 bp, Artificial Sequence
  Primer
gggcatcacttctaccaggtaa

SEQ ID NO: 14 - DNA, 22 bp, Artificial Sequence
  Primer
ccattttccttcttgtggagca

SEQ ID NO: 15 - DNA, 22 bp, Artificial Sequence
  Primer
agacatggagtcataggctctg

SEQ ID NO: 16 - DNA, 21 bp, Artificial Sequence
  Primer
tggtttgccatcgttttgctg

SEQ ID NO: 17 - DNA, 22 bp, Artificial Sequence
  Primer
acaggtgaggttcactgtttct

SEQ ID NO: 18 - protein, 15 aa, Artificial Sequence
  Immunogen
CYASINFQKQPEDRQ

SEQ ID NO: 19 - DNA, 23 bp, Artificial Sequence
  Primer
ctcaggaagaaagggcttatttc

SEQ ID NO: 20 - DNA, 24 bp, Artificial Sequence
  Primer
gaaccactgttccaatttccatcc

SEQ ID NO: 21 - DNA, 21 bp, Artificial Sequence
  RNAi oligonucleotide with 2′-deoxythymidines to enhance nuclease resistance
cacagaccauaaauccacgtt